SEQUENCE LISTING (63 sequences)

SEQ ID NO: 1 - protein, 549 aa, Homo sapiens
MPASAPPRRPRPPPPSLSLLLVLLGLGGRRLRAEPGDGAQTWARVSRPPAPEAAGLFQGTFPDGFLWAVGSAAYQTEGGWQQHGKGASIWDTFTHHPLAPPGDSRNASLPLGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALCFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGTFPCDFAWGVVDNYIQVSQLTKPISSLTKPYH

SEQ ID NO: 2 - protein, 550 aa, Mus musculus
MLARAPPRRPPRLVLLRLLLLHLLLLALRARCLSAEPGQGAQTWARFARAPAPEAAGLLHDTFPDGFLWAVGSAAYQTEGGWRQHGKGASIWDTFTHHSGAAPSDSPIVVAPSGAPSPPLSSTGDVASDSYNNVYRDTEGLRELGVTHYRFSISWARVLPNGTAGTPNREGLRYYRRLLERLRELGVQPVVTLYHWDLPQRLQDTYGGWANRALADHFRDYAELCFRHFGGQVKYWITIDNPYVVAWHGYATGRLAPGVRGSSRLGYLVAHNLLLAHAKVWHLYNTSFRPTQGGRVSIALSSHWINPRRMTDYNIRECQKSLDFVLGWFAKPIFIDGDYPESMKNNLSSLLPDFTESEKRLIRGTADFFALSFGPTLSFQLLDPNMKFRQLESPNLRQLLSWIDLEYNHPPIFIVENGWFVSGTTKRDDAKYMYYLKKFIMETLKAIRLDGVDVIGYTAWSLMDGFEWHRGYSIRRGLFYVDFLSQDKELLPKSSALFYQKLIEDNGFPPLPENQPLEGTFPCDFAWGVVDNYVQLSPLTKPSVGLLLPH

SEQ ID NO: 3 - DNA, 1650 bp, Homo sapiens
  source 1..1650: mol_type = other DNA
atgcccgccagcgccccgccgcgccgcccgcgtccaccgccgccgtcgctgtcgctgctgctggtgctgctgggcctgggcggacgacgcctgcgtgcggagccgggcgacggcgcgcagacctgggcccgtgtctcgcggcctcctgcccccgaggccgcgggcctcttccagggcaccttccccgacggcttcctctgggccgtgggcagcgccgcctaccagaccgagggcggctggcagcagcacggcaagggtgcgtccatctgggacacgttcacccaccaccccctggcacccccgggagactcccggaacgccagtctgccgttgggcgccccgtcgccgctgcagcccgccaccggggacgtagccagcgacagctacaacaacgtcttccgcgacacggaggcgctgcgcgagctcggggtcactcactaccgcttctccatctcgtgggcgcgagtgctccccaatggcagcgcgggcgtccccaaccgcgaggggctgcgctactaccggcgcctgctggagcggctgcgggagctgggcgtgcagcccgtggtcaccctgtaccactgggacctgccccagcgcctgcaggacgcctacggcggctgggccaaccgcgccctggccgaccacttcagggattacgcggagctctgcttccgccacttcggcggtcaggtcaagtactggatcaccatcgacaacccctacgtggtggcctggcacggctacgccaccgggcgcctggcccccggcatccggggcagcccgcggctcgggtacctggtggcgcacaacctcctcctggctcatgccaaagtctggcatctctacaatacttctttccgtcccactcagggaggtcaggtgtccattgccctaagctctcactggatcaatcctcgaagaatgaccgaccacagcatcaaagaatgtcaaaaatctctggactttgtactaggttggtttgccaaacccgtatttattgatggtgactatcccgagagcatgaagaataacctttcatctattctgcctgattttactgaatctgagaaaaagttcatcaaaggaactgctgacttttttgctctttgctttggacccaccttgagttttcaacttttggaccctcacatgaagttccgccaattggaatctcccaacctgaggcaactgctttcctggattgaccttgaatttaaccatcctcaaatatttattgtggaaaatggctggtttgtctcagggaccaccaagagagatgatgccaaatatatgtattacctcaaaaagttcatcatggaaaccttaaaagccatcaagctggatggggtggatgtcatcgggtataccgcatggtccctcatggatggtttcgagtggcacagaggttacagcatcaggcgtggactcttctatgttgactttctaagccaggacaagatgttgttgccaaagtcttcagccttgttctaccaaaagctgatagagaaaaatggcttccctcctttacctgaaaatcagcccctagaagggacatttccctgtgactttgcttggggagttgtcgacaactacattcaagtaagtcagctgacaaaaccaatcagcagtctcaccaagccctatcactag

SEQ ID NO: 4 - DNA, 1653 bp, Mus musculus
  source 1..1653: mol_type = other DNA
atgctcgcccgcgcccctcctcgccgcccgccgcggctggtgctgctccgtttgctgttgctgcatctgctgctgctcgccctgcgcgcccgctgcctgagcgctgagccgggtcagggcgcgcagacctgggctcgcttcgcgcgcgctcctgccccagaggccgctggcctcctccacgacaccttccccgacggtttcctctgggcggtaggcagcgccgcctatcagaccgagggcggctggcgacagcacggcaaaggcgcgtccatctgggacactttcacccatcactctggggcggccccgtccgactccccgatcgtcgtggcgccgtcgggtgccccgtcgcctcccctgtcctccactggagatgtggccagcgatagttacaacaacgtctaccgcgacacagaggggctgcgcgaactgggggtcacccactaccgcttctccatatcgtgggcgcgggtgctccccaatggcaccgcgggcactcccaaccgcgaggggctgcgctactaccggcggctgctggagcggctgcgggagctgggcgtgcagccggtggttaccctgtaccattgggacctgccacagcgcctgcaggacacctatggcggatgggccaatcgcgccctggccgaccatttcagggattatgccgagctctgcttccgccacttcggtggtcaggtcaagtactggatcaccattgacaacccctacgtggtggcctggcacgggtatgccaccgggcgcctggccccgggcgtgaggggcagctccaggctcgggtacctggttgcccacaacctacttttggctcatgccaaagtctggcatctctacaacacctctttccgccccacacagggaggccgggtgtctatcgccttaagctcccattggatcaatcctcgaagaatgactgactataatatcagagaatgccagaagtctcttgactttgtgctaggctggtttgccaaacccatatttattgatggcgactacccagagagtatgaagaacaacctctcgtctcttctgcctgattttactgaatctgagaagaggctcatcagaggaactgctgacttttttgctctctccttcggaccaaccttgagctttcagctattggaccctaacatgaagttccgccaattggagtctcccaacctgaggcagcttctgtcttggatagatctggaatataaccaccctccaatatttattgtggaaaatggctggtttgtctcgggaaccaccaaaagggatgatgccaaatatatgtattatctcaagaagttcataatggaaaccttaaaagcaatcagactggatggggtcgacgtcattgggtacaccgcgtggtcgctcatggacggtttcgagtggcataggggctacagcatccggcgaggactcttctacgttgactttctgagtcaggacaaggagctgttgccaaagtcttcggccttgttctaccaaaagctgatagaggacaatggctttcctcctttacctgaaaaccagccccttgaagggacatttccctgtgactttgcttggggagttgttgacaactacgttcagctgagtcctttgacaaaacccagtgtcggcctcttgcttcctcactaa

SEQ ID NO: 5 - DNA, 1002 bp, Homo sapiens
  source 1..1002: mol_type = other DNA
gcggccgcacccatgcctcctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgagataaccagggctgaaagaggcccgcctgggggctgcagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccggttcgaacaggtaagcgcccctaaaatccctttggcacaatgtgtcctgaggggagaggcagcgacctgtagatgggacgggggcactaaccctcagggtttggggttctgaatgtgagtatcgccatctaagcccagtatttggccaatctcagaaagctcctggctccctggaggatggagagagaaaaacaaacagctcctggagcagggagagtgttggcctcttgctctccggctccctctgttgccctctggtttctccccaggttcgaaggatcc

SEQ ID NO: 6 - DNA, 2687 bp, synthetic construct
  misc_feature 1..2687: note = Gene construct
  source 1..2687: mol_type = other DNA
gcggccgcacccatgcctcctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgagataaccagggctgaaagaggcccgcctgggggctgcagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccggttcgaacaggtaagcgcccctaaaatccctttggcacaatgtgtcctgaggggagaggcagcgacctgtagatgggacgggggcactaaccctcagggtttggggttctgaatgtgagtatcgccatctaagcccagtatttggccaatctcagaaagctcctggctccctggaggatggagagagaaaaacaaacagctcctggagcagggagagtgttggcctcttgctctccggctccctctgttgccctctggtttctccccaggttcgaaggatccactagtccagtgtggtggaattcgccgccaccatgctagcccgcgcccctcctcgccgcccgccgcggctggtgctgctccgtttgctgttgctgcatctgctgctgctcgccctgcgcgcccgctgcctgagcgctgagccgggtcagggcgcgcagacctgggctcgcttcgcgcgcgctcctgccccagaggccgctggcctcctccacgacaccttccccgacggtttcctctgggcggtaggcagcgccgcctatcagaccgagggcggctggcgacagcacggcaaaggcgcgtccatctgggacactttcacccatcactctggggcggccccgtccgactccccgatcgtcgtggcgccgtcgggtgccccgtcgcctcccctgtcctccactggagatgtggccagcgatagttacaacaacgtctaccgcgacacagaggggctgcgcgaactgggggtcacccactaccgcttctccatatcgtgggcgcgggtgctccccaatggcaccgcgggcactcccaaccgcgaggggctgcgctactaccggcggctgctggagcggctgcgggagctgggcgtgcagccggtggttaccctgtaccattgggacctgccacagcgcctgcaggacacctatggcggatgggccaatcgcgccctggccgaccatttcagggattatgccgagctctgcttccgccacttcggtggtcaggtcaagtactggatcaccattgacaacccctacgtggtggcctggcacgggtatgccaccgggcgcctggccccgggcgtgaggggcagctccaggctcgggtacctggttgcccacaacctacttttggctcatgccaaagtctggcatctctacaacacctctttccgccccacacagggaggccgggtgtctatcgccttaagctcccattggatcaatcctcgaagaatgactgactataatatcagagaatgccagaagtctcttgactttgtgctaggctggtttgccaaacccatatttattgatggcgactacccagagagtatgaagaacaacctctcgtctcttctgcctgattttactgaatctgagaagaggctcatcagaggaactgctgacttttttgctctctccttcggaccaaccttgagctttcagctattggaccctaacatgaagttccgccaattggagtctcccaacctgaggcagcttctgtcttggatagatctggaatataaccaccctccaatatttattgtggaaaatggctggtttgtctcgggaaccaccaaaagggatgatgccaaatatatgtattatctcaagaagttcataatggaaaccttaaaagcaatcagactggatggggtcgacgtcattgggtacaccgcgtggtcgctcatggacggtttcgagtggcataggggctacagcatccggcgaggactcttctacgttgactttctgagtcaggacaaggagctgttgccaaagtcttcggccttgttctaccaaaagctgatagaggacaatggctttcctcctttacctgaaaaccagccccttgaagggacatttccctgtgactttgcttggggagttgttgacaactacgttcagctgagtcctttgacaaaacccagtgtcggcctcttgcttcctcactaa

SEQ ID NO: 7 - DNA, 7821 bp, synthetic construct
  misc_feature 1..7821: note = Expression vector
  source 1..7821: mol_type = other DNA
cagcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagacatggctcgacagatctgcgcgcgatcgatatcagcgctttaaatttgcgcatgctagcgcggccgcacccatgcctcctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgagataaccagggctgaaagaggcccgcctgggggctgcagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccggttcgaacaggtaagcgcccctaaaatccctttggcacaatgtgtcctgaggggagaggcagcgacctgtagatgggacgggggcactaaccctcagggtttggggttctgaatgtgagtatcgccatctaagcccagtatttggccaatctcagaaagctcctggctccctggaggatggagagagaaaaacaaacagctcctggagcagggagagtgttggcctcttgctctccggctccctctgttgccctctggtttctccccaggttcgaaggatccactagtccagtgtggtggaattcgccgccaccatgctagcccgcgcccctcctcgccgcccgccgcggctggtgctgctccgtttgctgttgctgcatctgctgctgctcgccctgcgcgcccgctgcctgagcgctgagccgggtcagggcgcgcagacctgggctcgcttcgcgcgcgctcctgccccagaggccgctggcctcctccacgacaccttccccgacggtttcctctgggcggtaggcagcgccgcctatcagaccgagggcggctggcgacagcacggcaaaggcgcgtccatctgggacactttcacccatcactctggggcggccccgtccgactccccgatcgtcgtggcgccgtcgggtgccccgtcgcctcccctgtcctccactggagatgtggccagcgatagttacaacaacgtctaccgcgacacagaggggctgcgcgaactgggggtcacccactaccgcttctccatatcgtgggcgcgggtgctccccaatggcaccgcgggcactcccaaccgcgaggggctgcgctactaccggcggctgctggagcggctgcgggagctgggcgtgcagccggtggttaccctgtaccattgggacctgccacagcgcctgcaggacacctatggcggatgggccaatcgcgccctggccgaccatttcagggattatgccgagctctgcttccgccacttcggtggtcaggtcaagtactggatcaccattgacaacccctacgtggtggcctggcacgggtatgccaccgggcgcctggccccgggcgtgaggggcagctccaggctcgggtacctggttgcccacaacctacttttggctcatgccaaagtctggcatctctacaacacctctttccgccccacacagggaggccgggtgtctatcgccttaagctcccattggatcaatcctcgaagaatgactgactataatatcagagaatgccagaagtctcttgactttgtgctaggctggtttgccaaacccatatttattgatggcgactacccagagagtatgaagaacaacctctcgtctcttctgcctgattttactgaatctgagaagaggctcatcagaggaactgctgacttttttgctctctccttcggaccaaccttgagctttcagctattggaccctaacatgaagttccgccaattggagtctcccaacctgaggcagcttctgtcttggatagatctggaatataaccaccctccaatatttattgtggaaaatggctggtttgtctcgggaaccaccaaaagggatgatgccaaatatatgtattatctcaagaagttcataatggaaaccttaaaagcaatcagactggatggggtcgacgtcattgggtacaccgcgtggtcgctcatggacggtttcgagtggcataggggctacagcatccggcgaggactcttctacgttgactttctgagtcaggacaaggagctgttgccaaagtcttcggccttgttctaccaaaagctgatagaggacaatggctttcctcctttacctgaaaaccagccccttgaagggacatttccctgtgactttgcttggggagttgttgacaactacgttcagctgagtcctttgacaaaacccagtgtcggcctcttgcttcctcactaagggccgcttaatacgactcactatagggaccggtggcgcgcctgatcattcgaaggccggccgaattcacgcgtgcggccgcaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtctacaggccttcgcgattaattaagtttaaaccatatgatcgatacatgtgtttactccggaatattaataggcctaggatgcatatggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggaattccagacgattgagcgtcaaaatgtaggtatttccatgagcgtttttccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtctacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 8 - DNA, 7282 bp, synthetic construct
  misc_feature 1..7282: note = Expression vector
  source 1..7282: mol_type = other DNA
cagcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagacatggctcgacagatctgcgcgcgatcgatatcagcgctttaaatttgcgcatgctagcgcggccgcacccatgcctcctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgagataaccagggctgaaagaggcccgcctgggggctgcagacatgcttgctgcctgccctggcgaaggattggcaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccggttcgaacaggtaagcgcccctaaaatccctttggcacaatgtgtcctgaggggagaggcagcgacctgtagatgggacgggggcactaaccctcagggtttggggttctgaatgtgagtatcgccatctaagcccagtatttggccaatctcagaaagctcctggctccctggaggatggagagagaaaaacaaacagctcctggagcagggagagtgttggcctcttgctctccggctccctctgttgccctctggtttctccccaggttcgaaggatccagtgggacgtataggaactagcgaggcaccggcagttgggtcagagctactgttgtcacttcacaaggcattttcttcaacagcaagtcggaaatctcatgagcctaaggcagaatccacctgtggcctctggttacaacccacaggactgaaaatccttccagccacagcaactggtgaatttcctggtcaattgccacaagtcatgagctgaaccccacttgagtttcagttcaggcagaactctagagacgactagggcaagctagacagcgactgcagagccttttgttgcagcgtgagcagtcctcagctgttgacatcactggggagcaaacgaggaccaggagcggtgaaaggacagtgtctgctgcagattgtcgtagcacccaaggaacactccagaaagcctcctaagcagtaacaagtgtggcaaggtgtagcccagccaacagtggcatctgcgaggcgtcccctccttcctcccactaccccgtataccctgggacctgtgcactgaaggactcattctaaaggctgtgcccctgcagccgccagcctcactcactggctgcctgtgccagctagagatttctttcctctgaggctggctgagaggaccactccagtttcctggcccatccagcaaagaagatacacatcatgcacgtgtaaaatgaggaaccggtttattgaacagcttaaggagagcaaaaatagtggctttagctacattttttacacactgagcaggaaagtctaaaccatcccgttcccctgtaccccaaagagaacagggcttgctggaggccagtgccaagggcggagtcgtgctcgcagcagacttgaattaaccccatgtaggccggcgagcagttgcccgcgtgaaaacaccaccctcttctcctggctgagaagatcaaagctctttttttaccctcttttcagcaaaggacctatttgttttcaggcaggaggatgttaaacttgcagcctctgacacacggtggaacctgcagtgcttggagaaacggcacgcacacgtgaaaacatcatgcctactccaaagccttcttgttgctggcaggagggaagcttgagactttcccacgcatagtcgtgacccgcgtggccgtttctgctctcagcaacattctctagtgttccggcttcaagcagcgcttgtcaggtttgaagctagcctcgacctcgagacgcgtgcggccgcaagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtctacaggccttcgcgattaattaagtttaaaccatatgatcgatacatgtgtttactccggaatattaataggcctaggatgcatatggcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggaattccagacgattgagcgtcaaaatgtaggtatttccatgagcgtttttccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtctacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 9 - DNA, 24 bp, synthetic construct
  misc_feature 1..24: note = Forward primer
  source 1..24: mol_type = other DNA
tcataatggaaaccttaaaagcaa

SEQ ID NO: 10 - DNA, 20 bp, synthetic construct
  misc_feature 1..20: note = Reverse primer
  source 1..20: mol_type = other DNA
cactgggttttgtcaaagga

SEQ ID NO: 11 - DNA, 25 bp, synthetic construct
  misc_feature 1..25: note = Probe
  source 1..25: mol_type = other DNA
agaagagtcctcgccggatgctgta

SEQ ID NO: 12 - DNA, 20 bp, synthetic construct
  misc_feature 1..20: note = Forward primer
  source 1..20: mol_type = other DNA
atgggtacaagcgcgtcctg

SEQ ID NO: 13 - DNA, 21 bp, synthetic construct
  misc_feature 1..21: note = Reverse primer
  source 1..21: mol_type = other DNA
agccgcaaatgcagatggatc

SEQ ID NO: 14 - DNA, 25 bp, synthetic construct
  misc_feature 1..25: note = Probe
  source 1..25: mol_type = other DNA
tgtggagactgagtacaccttccca

SEQ ID NO: 15 - DNA, 6758 bp, synthetic construct
  misc_feature 1..6758: note = Expression vector
  source 1..6758: mol_type = other DNA
cagcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagacatggctcgacagatctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctctctggctaactagagaacccactgcttactggcttatcgaaattaatacgactcactatagggagacccaagctggctagaatgctcgcccgcgcccctcctcgccgcccgccgcggctggtgctgctccgtttgctgttgctgcatctgctgctgctcgccctgcgcgcccgctgcctgagcgctgagccgggtcagggcgcgcagacctgggctcgcttcgcgcgcgctcctgccccagaggccgctggcctcctccacgacaccttccccgacggtttcctctgggcggtaggcagcgccgcctatcagaccgagggcggctggcgacagcacggcaaaggcgcgtccatctgggacactttcacccatcactctggggcggccccgtccgactccccgatcgtcgtggcgccgtcgggtgccccgtcgcctcccctgtcctccactggagatgtggccagcgatagttacaacaacgtctaccgcgacacagaggggctgcgcgaactgggggtcacccactaccgcttctccatatcgtgggcgcgggtgctccccaatggcaccgcgggcactcccaaccgcgaggggctgcgctactaccggcggctgctggagcggctgcgggagctgggcgtgcagccggtggttaccctgtaccattgggacctgccacagcgcctgcaggacacctatggcggatgggccaatcgcgccctggccgaccatttcagggattatgccgagctctgcttccgccacttcggtggtcaggtcaagtactggatcaccattgacaacccctacgtggtggcctggcacgggtatgccaccgggcgcctggccccgggcgtgaggggcagctccaggctcgggtacctggttgcccacaacctacttttggctcatgccaaagtctggcatctctacaacacctctttccgccccacacagggaggccgggtgtctatcgccttaagctcccattggatcaatcctcgaagaatgactgactataatatcagagaatgccagaagtctcttgactttgtgctaggctggtttgccaaacccatatttattgatggcgactacccagagagtatgaagaacaacctctcgtctcttctgcctgattttactgaatctgagaagaggctcatcagaggaactgctgacttttttgctctctccttcggaccaaccttgagctttcagctattggaccctaacatgaagttccgccaattggagtctcccaacctgaggcagcttctgtcttggatagatctggaatataaccaccctccaatatttattgtggaaaatggctggtttgtctcgggaaccaccaaaagggatgatgccaaatatatgtattatctcaagaagttcataatggaaaccttaaaagcaatcagactggatggggtcgacgtcattgggtacaccgcgtggtcgctcatggacggtttcgagtggcataggggctacagcatccggcgaggactcttctacgttgactttctgagtcaggacaaggagctgttgccaaagtcttcggccttgttctaccaaaagctgatagaggacaatggctttcctcctttacctgaaaaccagccccttgaagggacatttccctgtgactttgcttggggagttgttgacaactacgttcagctgagtcctttgacaaaacccagtgtcggcctcttgcttcctcactaagggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggaattccagacgattgagcgtcaaaatgtaggtatttccatgagcgtttttccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtctacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 16 - DNA, 8046 bp, synthetic construct
  misc_feature 1..8046: note = Expression vector
  source 1..8046: mol_type = other DNA
cagcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagacatggctcgacagatctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagccagcacatgactgacactcggaagcacagaacaaagggtaggacggtgcccatgggtcaggctgtagccacgccaccctttccaccctgtcctagccagaggcagcaatgtgctccatacagatcctcctaacacacccacactgtcggtccccagcacgcagatgcccgacagccccttaggcaaatggcttagctgactgccccaccacacgccgtcgccatgcagtccagtggggagtcggaggcagcctccttcctgcctctcctcggcctgcacgtgtccccccaccaggcagagacccttctacaccccgggtgtctgcggtcacatcgcggtggggcatgcagctgttggccttcgagcatgttttgttttccttggccagtgtctccagagaaacgcacgtgggtttgtgtccagcggtccatctctgcaacagttgttcctttgggattggatgctaggaggtcacgggagaggtgtccatccaaagcagtgtctgtgtcacacactgtccccacacacagggccacctctgcacagactcccccgactcgattctgggcacagagctcagtgaccttccagagactgccacgaaccggtgatgcctccacgcttgagacatcctgaccgcagggcccaaggcgcactggctcagggggtgacagtgaggggtctgcaaacagactgctgatgctcaacccggccgctgccgagctgtgtgacttgggcacgtcacttaacctctctcggcctctgtctcctcccggggataagagtagtagcacctgcttcccggggctgtgaggatccagtgggacgtataggaactagcgaggcaccggcagttgggtcagagctactgttgtcacttcacaaggcattttcttcaacagcaagtcggaaatctcatgagcctaaggcagaatccacctgtggcctctggttacaacccacaggactgaaaatccttccagccacagcaactggtgaatttcctggtcaattgccacaagtcatgagctgaaccccacttgagtttcagttcaggcagaactctagagacgactagggcaagctagacagcgactgcagagccttttgttgcagcgtgagcagtcctcagctgttgacatcactggggagcaaacgaggaccaggagcggtgaaaggacagtgtctgctgcagattgtcgtagcacccaaggaacactccagaaagcctcctaagcagtaacaagtgtggcaaggtgtagcccagccaacagtggcatctgcgaggcgtcccctccttcctcccactaccccgtataccctgggacctgtgcactgaaggactcattctaaaggctgtgcccctgcagccgccagcctcactcactggctgcctgtgccagctagagatttctttcctctgaggctggctgagaggaccactccagtttcctggcccatccagcaaagaagatacacatcatgcacgtgtaaaatgaggaaccggtttattgaacagcttaaggagagcaaaaatagtggctttagctacattttttacacactgagcaggaaagtctaaaccatcccgttcccctgtaccccaaagagaacagggcttgctggaggccagtgccaagggcggagtcgtgctcgcagcagacttgaattaaccccatgtaggccggcgagcagttgcccgcgtgaaaacaccaccctcttctcctggctgagaagatcaaagctctttttttaccctcttttcagcaaaggacctatttgttttcaggcaggaggatgttaaacttgcagcctctgacacacggtggaacctgcagtgcttggagaaacggcacgcacacgtgaaaacatcatgcctactccaaagccttcttgttgctggcaggagggaagcttgagactttcccacgcatagtcgtgacccgcgtggccgtttctgctctcagcaacattctctagtgttccggcttcaagcagcgcttgtcaggtttgaagctagcctcgacctcgagacgcgtgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacgcggccgcttccctttagtgagggttaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataagggactagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggaattccagacgattgagcgtcaaaatgtaggtatttccatgagcgtttttccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtctacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 17 - protein, 38 aa, synthetic construct
SQLTASVSSPPTRALSLASSAFLLGWRSWRILCPEPTR

SEQ ID NO: 18 - protein, 770 aa, synthetic construct
  REGION 1..770: note = AAV9_P1 capsid polypeptide, insertion site 588
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQGQSGRGDLGLSAQAAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNLEFAVNTEGVYSEPRPIGTRYLTRNL

SEQ ID NO: 19 - protein, 745 aa, synthetic construct
  REGION 1..745: note = AAV9LD_P1 capsid polypeptide, insertion site 588
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWAAASSWALNGRNSLMNPGPAMASHKEGEDRFFPLSGSLIFGKQGTGRDNVDADKVMITNEEEIKTTNPVATESYGQVATNHQGQSGRGDLGLSAQAAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 20 - protein, 745 aa, synthetic construct
  REGION 1..745: note = AAV9_P1 capsid polypeptide, insertion site 589
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSGQSGRGDLGLSAQAATGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 21 - protein, 745 aa, synthetic construct
  REGION 1..745: note = AAV9_P3 capsid polypeptide, insertion site 588
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQGQSGRGDAVGVAQAAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 22 - protein, 745 aa, synthetic construct
  REGION 1..745: note = AAV9_P3 capsid polypeptide 2, insertion site 589
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSGQSGRGDAVGVAQAATGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 23 - protein, 745 aa, synthetic construct
  REGION 1..745: note = AAV9LD_P1 capsid polypeptide 2, insertion site 589
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWAAASSWALNGRNSLMNPGPAMASHKEGEDRFFPLSGSLIFGKQGTGRDNVDADKVMITNEEEIKTTNPVATESYGQVATNHQSGQSGRGDLGLSAQAATGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 24 - protein, 745 aa, synthetic construct
  REGION 1..745: note = AAV9LD_P3 capsid polypeptide, insertion site 588
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWAAASSWALNGRNSLMNPGPAMASHKEGEDRFFPLSGSLIFGKQGTGRDNVDADKVMITNEEEIKTTNPVATESYGQVATNHQGQSGRGDAVGVAQAAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 25 - protein, 745 aa, synthetic construct
  REGION 1..745: note = AAV9LD_P3 capsid polypeptide 2, insertion site 589
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWAAASSWALNGRNSLMNPGPAMASHKEGEDRFFPLSGSLIFGKQGTGRDNVDADKVMITNEEEIKTTNPVATESYGQVATNHQSGQSGRGDAVGVAQAATGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 26 - protein, 744 aa, synthetic construct
  REGION 1..744: note = AAVS10_P1 capsid polypeptide
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGNASGNWHCDSQWLGDRVITTSTRTWALPTYNNHLYKQISSASTGATNDNTYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQGQSGRGDLGLSAQAAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 27 - protein, 746 aa, synthetic construct
  REGION 1..746: note = AAVS1_P1 capsid polypeptide
MAADGYLPDWLEDNLSEGIREWWALKPGAPQPKANQQHQDNARGLVLPGYKYLGPGNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKTGQQPAKKRLNFGQTGDTESVPDPQPIGEPPAAPAAVGPTTMASGGGAPMADNNEGADGVGNASGNWHCDSTWLGDRVITTSTRTWALPTYNNHLYKQISNGTSGGATNDNTYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQGQSGRGDLGLSAQAAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 28 - protein, 745 aa, synthetic construct
  REGION 1..745: note = AAVD20_P1 capsid polypeptide
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQTKKRVLEPFGLVEEGAKTAPGKKRPVEQSPQEPDSSSGIVKTGQQPAKKRLNFGQTGDTESVPDPQPLGEPPATPSGVGSLTMASGGGAPMADNNEGADGVGNASGNWHCDSTWLGDRVITTSTRTWALPTYNNHLYKQISNGTSGGATNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQGQSGRGDLGLSAQAAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 29 - protein, 745 aa, synthetic construct
  REGION 1..745: note = AAVH15_P1 capsid polypeptide
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQGQSGRGDLGLSAQAAQTGWVQNQGILPGMVWQDRDVYLQGPIWAKIPHTDGNFHPSPLMGGFGLKHPPPQILIKNTPVPADPPAEFSATKFASFITQYSTGQVSVEIEWELQKENSKRWNPEVQYTSNYAKSANVDFTVDNNGLYTEPRPIGTRYLTRPL

SEQ ID NO: 30 - DNA, 2238 bp, synthetic construct
  misc_feature 1..2238: note = AAV9_P1 capsid polypeptide encoding, insertion site amino acid 588
  source 1..2238: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagggccagagtggccgcggcgatctgggcctgagcgcccaggcggcccagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaaacacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 31 - DNA, 2238 bp, synthetic construct
  misc_feature 1..2238: note = AAV9LD_P1 capsid polypeptide encoding, insertion site amino acid 588
  source 1..2238: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttgggctgcagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagggccagagtggccgcggcgatctgggcctgagcgcccaggcggcccagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaaacacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 32 - DNA, 2238 bp, synthetic construct
  misc_feature 1..2238: note = AAV9_P1 capsid polypeptide 2 encoding, insertion site amino acid 589
  source 1..2238: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtggccagagtggccgcggcgatctgggcctgagcgcccaggcggccaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaaacacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 33 - DNA, 2238 bp, synthetic construct
  misc_feature 1..2238: note = AAV9_P3 capsid polypeptide encoding, insertion site 588
  source 1..2238: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagggccagagtggccgcggcgatgcggtgggcgtggcccaggcggcccagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaaacacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 34 - DNA, 2238 bp, synthetic construct
  misc_feature 1..2238: note = AAV9_P3 capsid polypeptide 2 encoding, insertion site 589
  source 1..2238: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtggccagagtggccgcggcgatgcggtgggcgtggcccaggcggccaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaaacacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 35 - DNA, 2238 bp, synthetic construct
  misc_feature 1..2238: note = AAV9LD_P1 capsid polypeptide 2 encoding, insertion site amno acid 589
  source 1..2238: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttgggctgcagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtggccagagtggccgcggcgatctgggcctgagcgcccaggcggccaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaaacacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 36 - DNA, 2238 bp, synthetic construct
  misc_feature 1..2238: note = AAV9LD_P3 capsid polypeptide encoding, insertion site amino acid 588
  source 1..2238: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttgggctgcagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagggccagagtggccgcggcgatgcggtgggcgtggcccaggcggcccagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaaacacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 37 - DNA, 2238 bp, synthetic construct
  misc_feature 1..2238: note = AAV9LD_P3 capsid polypeptide 2 encoding, insertion site amino acid 589
  source 1..2238: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttgggctgcagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtggccagagtggccgcggcgatgcggtgggcgtggcccaggcggccaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaaacacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 38 - DNA, 2235 bp, synthetic construct
  misc_feature 1..2235: note = AAVS10_P1 capsid polypeptide encoding
  source 1..2235: mol_type = other DNA
atggctgccgatggttatcttccagattggctcgaggacaacctctctgagggcattcgcgagtggtgggctttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaaaaagaggcttcttgaacctcttggtctggttgaggaagcggctaagacggctcctggaaagaaacgtccggtagagcagtcgccacaagagccagactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggcgccgacggagtgggtaatgcctcgggaaattggcattgcgattcccaatggctgggggacagagtcatcaccaccagcacccgaacctgggccctgcccacctacaacaatcacctctacaagcaaatctccagtgcttcaacgggggccaccaacgacaacacctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagggccagagtggccgcggcgatctgggcctgagcgcccaggcggcccagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaaacacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 39 - DNA, 2241 bp, synthetic construct
  misc_feature 1..2241: note = AAVS1_P1 capsid polypeptide encoding
  source 1..2241: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagccatcaccccagcgttctccagactcctctacgggcatcggcaagacaggccagcagcccgctaaaaagagactcaatttcggtcagactggcgacacagagtcagtcccagaccctcaaccaatcggagaacctcccgcagcccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgatttcaacagattccactgccatttctcaccacgtgactggcagcgactcatcaacaacaattggggattccggcccaagagactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagggccagagtggccgcggcgatctgggcctgagcgcccaggcggcccagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaaacacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 40 - DNA, 2238 bp, synthetic construct
  misc_feature 1..2238: note = AAVD20_P1 capsid polypeptide encoding
  source 1..2238: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccagaccaagaagagggttctcgaaccttttggtctggttgaggaaggtgctaagacggctcctggaaagaaacgtccggtagagcagtcgccacaagagccagactcctcctcgggcattgtcaagacaggccagcagcccgctaaaaagagactcaattttggtcagactggcgacacagagtcagtccccgacccacaacctctcggagaacctccagcaaccccctcaggtgtgggatctcttacaatggcttcaggcggtggcgcaccaatggcagacaataacgaaggcgccgacggagtgggtaatgcctcaggaaattggcattgcgattccacatggctgggcgacagagtcatcaccaccagcacccgaacctgggccctgcccacctacaacaaccacctctacaagcaaatctccaacgggacatcgggaggagccaccaacgacaacgcctacttcggctacagcaccccctgggggtattttgactttaacagattccactgccacttttcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagggccagagtggccgcggcgatctgggcctgagcgcccaggcggcccagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaaacacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 41 - DNA, 2238 bp, synthetic construct
  misc_feature 1..2238: note = AAVH15_P1 capsid polypeptide encoding
  source 1..2238: mol_type = other DNA
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtaccttcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagggccagagtggccgcggcgatctgggcctgagcgcccaggcggcccagaccggctgggttcaaaaccaaggaatacttccgggtatggtttggcaggacagagatgtgtacctgcaaggacccatttgggccaaaattcctcacacggacggcaacttccacccgtctccgctgatgggcggctttggcctgaaacatcctccgcctcagatcctgatcaagaacacgcctgtacctgcggatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaa

SEQ ID NO: 42 - protein, 1012 aa, Pan paniscus
MPASAPPRRPRPPPPSLSLLLVLLALGGRRLRAEPGDGAQTWARFSRPPAPEAAGLFQGTFPDGFLWAVGSAAYQTEGGWQQHGKGASIWDTFTHHPLAPPGDSRNASLPSGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALSFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGTFPCDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILEYYCCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDSNGFPGPETLERFCPEEFTVCTECSFFHTRKSLLAFIAFLFFAFIISLSLIFYYSKKGRRSYK

SEQ ID NO: 43 - protein, 1012 aa, Pan troglodytes
MPASAPPRRPRPPPPSLSLLLVLLALGGRRLRAEPGDGAQTWARFSRPPAPEAAGLFQGTFPDGFLWAVGSAAYQTEGGWQQHGKGASIWDTFTHHPLAPPGDSRNASLPSGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTEHSIKECQKSLDFVLGWFAKPVFIDGDYPESMKNNLSSILPDFTESEKKFIKGTADFFALSFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGTFPCDFAWGVVDNYIQVDTTLSQFTDLNVYLWDVHHSKRLIKVDGVVTKKRKSYCVDFAAIQPQIALLQEMHITHFRFSLDWALILPLGNQSQVNHTILQYYRCMASELVRVNITPVVALWQPMAPNQGLPRLLARQGAWENPYTALAFAEYARLCFQALGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAQILDGINLCGYFAYSFNDRTAPRFGLYRYAADQFEPKASMKHYRKIIDSNGFPGPETLERFCPEEFTVCTECSFFHTRKSLLAFIAFLFFAFIISLSLIFYYSKKGRRSYK

SEQ ID NO: 44 - protein, 1012 aa, Gorilla gorilla
MPASAPPRRPRPPPPSLSLLLVLLALGGRRLRAEPGDGAQTWARFSRPPAPEAAGLFQGTFPDGFLWAVGSAAYQTEGGWQQHGKGESIWDTFTHHPLAPPGDSRNASLPSGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALSFGPTLSFQLLDPHMKFRQLESPNLRQLLSWIDLEFNHPQIFIVENGWFVSGTTKRDDAKYMYYLKKFIMETLKAIKLDGVDVIGYTAWSLMDGFEWHRGYSIRRGLFYVDFLSQDKTLLPKSSALFYQKLIEKNGFPPLPENQPLEGTFPCDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDSNGFPGPETLERFCPEEFTVCTECSFFHTRKSLLAFIAFLFFAFIISLSLIFYYSKKGRRSYK

SEQ ID NO: 45 - protein, 551 aa, Macaca fascicularis
MPASAPPRRPRPPPPSLSLSLLLVLLGLAGRRLRAEPGDGAQTWARFARPPAPEAAGLFQGTFPDGFLWAVGSAAYQTEGGWQQHGKGASIWDTFTHHPLAPPGDSRIANVPSGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPIFIDGDYPESMKNNLSSLLPDFTESEKKFIKGTADFFALSFGPTLSFQLLDPHMKFRQLESPSLRQLLSWIDLEYNHPQIFIVENGWFVSGTTKRDDAKYMYYLKKFIMETLKAIKLDGVDVIGYTAWSLMDGFEWHRGYSIRRGLFYVDFLSQEKTLLPKSSALFYQKLIEKNGFPPLPENQPLEGTFPCDFAWGIVDNYIQVSQLAKPISSLTKPYH

SEQ ID NO: 46 - protein, 1012 aa, Pongo abelii
MPASAPPCRPRPPPPSLSLLLVLLALGGRRLRAEPGDGAQTWARFARPPAPEAAGLFQGTFPDGFLWAVGSAAYQTEGGWQQHGKGASIWDTFTHHPLAPPGDSRNANLPSGAPSPLQPATGDVASDSYNNVFRDTEALRKLGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALSFGPTLSFQLLDPHMKFRQLESPNLRQLLSWIDLEYNHPQIFIVENGWFVSGTTKRDDAKYMYYLKKFIMETLKAIKLDGVDVIGYTAWSLMDGFEWHRGYSIRRGLFYVDFLSQDKMLLPKSSALFYQKLIEKNGFPPLPENQPLEGTFPCDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPCLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPVKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDSNGFPGPETLERFCPEEFTMCTECSFFHTRKSLLAFIAFLFFAFIISLSLIFYYSKKGRRSYK

SEQ ID NO: 47 - protein, 1012 aa, Pongo pygmaeus
MPASAPPCRPRPPPPSLSLLLVLLALGGRRLRAEPGDGAQTWARFARPPAPEAAGLFQGTFPDGFLWAVGSAAYQTEGGWQQHGKGASIWDTFTHHPLAPPGDSRNANLPSGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALSFGPTLSFQLLDPHMKFRQLESPNLRQLLSWIDLEYNHPQIFIVENGWFVSGTTKRDDAKYMYYLKKFIMETLKAIKLDGVDVIGYTAWSLMDGFEWHRGYSIRRGLFYVDFLSQDKTLLPKSSALFYQKLIEKNGFPPLPENQPLEGTFPCDFAWGVVDNYIQVDTTLSQFTDLNVYLWDVHHSKRLIKVDGVVTKKRKSYCVDFAAIQPQIALLQEMHVTHFRFSLDWALILPLGNQSQVNHTILQYYRCMASELVRVNITPVVALWQPMAPNQGLPCLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPVKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRYAADQFEPKASMKHYRKIIDSNGFPGPETLERFCPEEFTMCTECSFFHTRKSLLAFIAFLFFAFIISLSLIFYYSKKGRRSYK

SEQ ID NO: 48 - protein, 1012 aa, Piliocolobus tephrosceles
MPASAPPRRPRPPPPSLSLLLVLLALGGRRLRAEPGDGAQTWARFARPPAPEAAGLFQGTFPDGFLWAVGSAAYQTEGGWQQHGKGASIWDTFTHHPLAPPGDSRIVNLPSGAPSPPQPATADVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWISPRRMTDHSIKECQKSMDFVLGWFAKPIFIDGDYPESMKNNLSSLLPDFTEPEKKFIKGTADFFALSFGPTLSFQLLDPHMKFRQLESPSLRQLLSWIDLEYNHPQIFIVENGWFVSGTTKRDDAKYMYYLKKFIMETLKAIKLDGVDVIGYTAWSLMDGFEWHRGYSIRRGLFYVDFLSQEKMLLPKSSALFYQKLIEKNGFPPLPENQPLEGTFPCDFAWGIVDNYIQVDTTLSQFTDANVYLWDVHHSKRLIKVDGVVTKKRKSYCVDFATIQPQIALLQEMHVTHFRFSLDWALILPLGNQSQVNHTILQYYRCMVSELVRVNITPVVALWQPVAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLSSPSQVAVVPWGLRKVLNWLKFKYGDLPMYIISNGIDDGLHAEDDQLRVYYMQNYINEALKAHILDGINLCGYFAYSFNDRTAPRFGLYRFAADQFEPKPSMKHYRKIIDSNGFPGPETLEKFCPEEFTMCTECSFFHTRKPLVAFIAFLFLAFIVSLSLIFYYSKKGRRRYK

SEQ ID NO: 49 - protein, 1012 aa, Pongo abelii
MPASAPPRRPRPPPPSLSLLLVLLALGGRRLRAEPGDGAQTWARFARPPAPEAAGLFQGTFPDGFLWAAGSAAYQTEGGWQQHGKGASIWDTFTHHPLAPPGDSRNANLPSGAQSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWISPRRMTDHSIKECQKSLDFVLGWFAKPVFIDGDYPESMKNNLSSILPDFTESEKKFIKGTADFFALSFGPTLSFQLLDPHMKFRQLESPNLRQLLSWIDLEYNHPQIFIVENGWFVSGTTKRDDAKYMYYLKKFIMETLKAIKLDGVDVIGYTAWSLMDGFEWHRGYSIRRGLFYVDFLSQDKTLLPKSSALFYQKLIEKNGFPPLPENQPLEGTFPCDFAWGVVDNYIQVDTTLSQFTDLNVYLWDVYHSKRLIKVDGVVTKKRKPYCVDFAAIQPQIALLQEMHVTHFRFSLDWALIVPLGNQSQVNHTILQYYRCMASELVRVNITPVVALWQPMAPNQGLPHLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAIVPWGLRKVLNWLKFKYGDLPMYIISNGIDDGLHAEDDQLRVYYMQNYINEALKAHILDGINLCGYFAYSFNDRTAPRFGLYHYAADQFEPKASMKHYRKIIDSNGFLGPETLERVCPEEFTMCTECSFFHTRKSLLAFIAFLFFAFIISLSLIFYYSKKGRRSYK

SEQ ID NO: 50 - protein, 1012 aa, Nomascus leucogenys; identical to SEQ ID NO: 49

SEQ ID NO: 51 - protein, 1014 aa, Macaca thibetana
MPASAPPRRPRPPPPSLSLSLLLVLLALGGRRLRAEPGDGAQTWARFARPPAPEAAGLFQGTFPDGFLWAVGSAAYQTEGGWQQHGKGASIWDTFTHHPLAPPGDSRIANVPSGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPIFIDGDYPESMKNNLSSLLPDFTESEKKFIKGTADFFALSFGPTLSFQLLDPHMKFRQLESPSLRQLLSWIDLEYNHPQIFIVENGWFVSGTTKRDDAKYMYYLKKFIMETLKAIKLDGVDVIGYTAWSLMDGFEWHRGYSIRRGLFYVDFLSQEKTLLPKSSALFYQKLIEKNGFPPLPENQPLEGTFPCDFAWGIVDNYIQVDTTLSQFTDLNVYLWDVHHSKRLIKVDGVVTKKRKSYCVDFAAIQPQITLLQEMHVTHFRFSLDWALILPLGNQSQVNHTILQYYRCMVSELVRVNITPVVALWQPVAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINVCGYFAYSFNDRTAPRFGLYRFAADQFEPKPSMKHYRKIIDSNGFPGPETLEKFCPEEFTVCTECSFFHTRKPLVAFIAFLFFAFIVSLSLIFYYSKKGRRRYK

SEQ ID NO: 52 - protein, 1014 aa, Macaca nemestrina
MPASAPPRRPRPPPPSLSLSLLLVLLALGGRRLRAEPGDGAQTWARFARPPAPEAAGLFQGTFPDGFLWAVGSAAYQTEGGWQQHGKGASIWDTFTHHPLAPPGDSRIANVPSGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPIFIDGDYPESMKNNLSSLLPDFTESEKKFIKGTADFFALSFGPTLSFQLLDPHMKFRQLESPSLRQLLSWIDLEYNHPQIFIVENGWFVSGTTKRDDAKYMYYLKKFIMETLKAIKLDGVDVIGYTAWSLMDGFEWHRGYSIRRGLFYVDFLSQEKTLLPKSSALFYQKLIEKNGFPPLPENQPLEGTFPCDFAWGIVDNYIQVDTTLSQFTDLNVYLWDVHHSKRLIKVDGVVTKKRKSYCVDFAAIQPQITLLQEMHVTHFRFSLDWALILPLGNQSQVNHTILQYYRCMVSELVRVNITPVVALWQPVAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRFAADQFEPKPSMKHYRKIIDSNGFPGPETLEKFCPEEFTVCTECSFFHTRKPLVAFIAFLFFAFIVSLSLIFYYSKKGRRRYK

SEQ ID NO: 53 - protein, 962 aa, Macaca mulatta
PEAAGLFQGTFPDGFLWAVGSAAYQTEGGWQQHGKGASIWDTFTHHPLAPPGDSRIANVPSGAPSPLQPATGDVASDSYNNVFRDTEALRELAVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPIFIDGDYPESMKNNLSSLLPDFTESEKKFIKGTADFFALSFGPTLSFQLLDPHMKFRQLESPSLRQLLSWIDLEYNHPQIFIVENGWFVSGTTKRDDAKYMYYLKKFIMETLKAIKLDGVDVIGYTAWSLMDGFEWHRGYSIRRGLFYVDFLSQEKTLLPKSSALFYQKLIEKNGFPPLPENQPLEGTFPCDFAWGIVDNYIQVDTTLSQFTDLNVYLWDVHHSKRLIKVDGVVTKKRKSYCVDFAAIQPQITLLQEMHVTHFRFSLDWALILPLGNQSQVNHTILQYYRCMVSELVRVNITPVVALWQPVAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRFAADQFEPKPSMKHYRKIIDSNGFPGPETLEKFCPEELTVCTECSFFHTRKPLVAFIAFLFFAFIVSLSLIFYYSKKGRRRYK

SEQ ID NO: 54 - protein, 1014 aa, Macaca fascicularis
MPASAPPRRPRPPPPSLSLSLLLVLLGLAGRRLRAEPGDGAQTWARFARPPAPEAAGLFQGTFPDGFLWAVGSAAYQTEGGWQQHGKGASIWDTFTHHPLAPPGDSRIANVPSGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPIFIDGDYPESMKNNLSSLLPDFTESEKKFIKGTADFFALSFGPTLSFQLLDPHMKFRQLESPSLRQLLSWIDLEYNHPQIFIVENGWFVSGTTKRDDAKYMYYLKKFIMETLKAIKLDGVDVIGYTAWSLMDGFEWHRGYSIRRGLFYVDFLSQEKTLLPKSSALFYQKLIEKNGFPPLPENQPLEGTFPCDFAWGIVDNYIQVDTTLSQFTDLNVYLWDVHHSKRLIKVDGVVTKKRKSYCVDFAAIQPQITLLQEMHVTHFRFSLDWALILPLGNQSQVNHTILQYYRCMVSELVRVNITPVVALWQPVAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRFAADQFEPKPSMKHYRKIIDSNGFPGPETLEKFCPEEFTVCTECSFFHTRKPLVAFIAFLFFAFIVSLSLIFYYSKKGRRRYQ

SEQ ID NO: 55 - protein, 1005 aa, Hylobates moloch
MPASAPPRRPRPPPPSLSLLLVLLALGGRRLRAEPGDGAQTWARFARPPAPEAAGLFQGTFPDGFLWAAGSAAYQTEGGWQQHGKGASIWDTFTHHPLAPPGDSRNASLPSGATGDVAGDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSILPDFTESEKKFIKGTADFFALSFGPTLSFQLLDPHMKFRQLESPNLRQLLSWIDLEYNHPQIFIVENGWFVSGTTKRDDAKYMYYLKKFIMETLKAIKLDGVDVIGYTAWSLMDGFEWHRGYSIRRGLFYVDFLSQDKTLLPKSSALFYQKLIEKNGFPPLPENQPLEGTFPCDFAWGVVDNYIQVDTTLSQFTDLNVYLWDVHHSKRLIKVDGVVTKRRKPYCVDFAAIQPQIALLQEMHVTHFRFSLDWALIVPLGNQSQVNHTILQYYRCMASELVRVNITPVVALWQPMAPNQGLPHLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAIVPWGLRKVLNWLKFKYGDLPMYIISNGIDDGLHAEDDQLRVYYMQNYINEALKAHILDGINLCGYFAYSFNDRTAPRFGLYRYAADEFEPKASMKHYRNIIDSNGFLGPETLERVCPEEFTMCTECSFFHTRKSLLAFIAFLFFAFIISLSLIFYYSKKGRRSYK

SEQ ID NO: 56 - protein, 1014 aa, Macaca fascicularis
MPASAPPRRLRPSPPSLSLSLLLVLLALGGRRLRAEPGDGAQTWARFARPPAPEAAGLFQGTFPDGFLWAVGSAAYQTEGGWQQHGKGASIWDTFTHHPLAPPGDSRIANVPSGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPIFIDGDYPESMKNNLSSLLPDFTESEKKFIKGTADFFALSFGPTLSFQLLDPHMKFRQLESPSLRQLLSWIDLEYNHPQIFIVENGWFVSGTTKRDDAKYMYYLKKFIMETLKAIKLDGVDVIGYTAWSLMDGFEWHRGYSIRRGLFYVDFLSQEKTLLPKSSALFYQKLIEKNGFPPLPENQPLEGTFPCDFAWGIVDNYIQVDTTLSQFTDLNVYLWDVHHSKRLIKVDGVVTKKRKSYCVDFAAIQPQITLLQEMHVTHFRFSLDWALILPLGNQSQVNHTILQYYRCMVSELVRVNITPVVALWQPVAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRFAADQFEPKPSMKHYRKIIDSNGFPGPETLEKFCPEEFTVCTECSFFHTRKPLVAFIAFLFFAFIVSLSLIFYYSKKGRRRYK

SEQ ID NO: 57 - protein, 1014 aa, Macaca mulatta
MPASAPPRRLRPSPPSLSLSLLLVLLALGGRRLRAEPGDGAQTWARFARPPAPEAAGLFQGTFPDGFLWAVGSAAYQTEGGWQQHGKGASIWDTFTHHPLAPPGDSRIANVPSGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPIFIDGDYPESMKNNLSSLLPDFTESEKKFIKGTADFFALSFGPTLSFQLLDPHMKFRQLESPSLRQLLSWIDLEYNHPQIFIVENGWFVSGTTKRDDAKYMYYLKKFIMETLKAIKLDGVDVIGYTAWSLMDGFEWHRGYSIRRGLFYVDFLSQEKTLLPKSSALFYQKLIEKNGFPPLPENQPLEGTFPCDFAWGIVDNYIQVDTTLSQFTDLNVYLWDVHHSKRLIKVDGVVTKKRKSYCVDFAAIQPQITLLQEMHVTHFRFSLDWALILPLGNQSQVNHTILQYYRCMVSELVRVNITPVVALWQPVAPNQGLPRLLARQGAWENPYTALAFAEYARLCFQELGHHVKLWITMNEPYTRNMTYNAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRFAADQFEPKPSMKHYRKIIDSNGFPGPETLEKFCPEEFTVCTECSFFHTRKPLVAFIAFLFFAFIVSLSLIFYYSKKGRRRYK

SEQ ID NO: 58 - protein, 1012 aa, Theropithecus gelada
MPASAPPRRPRPPPPSLSLLLVLLALGGRRLRAEPGDGAQTWARFARPSAPEAAGLFQGTFPDGFLWAVGSAAYQTEGGWQQHGKGASIWDTFTHHPLAPPGDSRIANVPSGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPIFIDGDYPESMKNNLSSLLPDFTESEKKFIKGTADFFALSFGPTLSFQLLDPHMKFRQLESPSLRQLLSWIDLEYNHPQIFIVENGWFVSGTTKRDDAKYMYYLKKFIMETLKAIKLDGVDVIGYTAWSLMDGFEWHRGYSIRRGLFYVDFLSQEKTLLPKSSALFYQKLIEKNGFPPLPENQPLEGTFPCDFAWGIVDNYIQVDTTLSQFTDLNVYLWDVHHSKRLIKVDGVVTKKRKSYCVDFAAIQPQITLLQEMHITHFRFSLDWALILPLGNQSQVNHTILQYYRCMVSELVRVNITPVVALWQPVAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPVKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRFAADQFEPKPSVKHYRKIIDSNGFPGPETLETFCPEESTVCTECSFFHTRKPLVAFIAFLFFAFIVSLSLIFYYSKKGRRCYK

SEQ ID NO: 59 - protein, 1014 aa, Macaca fascicularis
MPASAPPRRPRPPPPSLSLSLLLVLLALGGCRLRAEPGDGAQTWARFARPPAPEAAGLFQGTFPDGFLWAVGSAAYQTEGGWQQHGKGASIWDTFTHHPLAPPGDSRIANVPSGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPIFIDGDYPESMKNNLSSLLPDFTESEKKFIKGTADFFALSFGPTLSFQLLDPHMKFRQLESPSLRQLLSWIDLEYNHPQIFIVENGWFVSGTTKRDDAKYMYYLKKFIMETLKAIKLDGVDVIGYTAWSLMDGFEWHRGYSIRRGLFYVDFLSQEKTLLPKSSALFYQKLIEKNGFPPLPENQPLEGTFPCDFAWGIVDNYIQVDTTLSQFTDLNVYLWDVHHSKRLIKVDGVVTKKRKSYCVDFAAIQPQITLLQEMHVTHFRFSLDWALILPLGNQSQVNHTILQYYRCMVSELVRVNITPVVALWQPVAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRFAADQFEPKPSMKHYRKIIDSNGFPGPETLEKFCPEEFTVCTECSFFHTRKPLVAFIAFLFFAFIVSLSLIFYYSKKGRRRYK

SEQ ID NO: 60 - protein, 1012 aa, Rhinopithecus roxellana
MPASAPPRRPRPPPPSLSLLLVLLALGGRRLRAETGDGAQTWARFARPPAPEAAGLFQGTFPDGFLWAVGSAAYQTEGGWQQHGKGASIWDTFTHHPLAPPGDSRIANLPSGAPSPPQPATADVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPVFIDGDYPESMKNNLSSLLPDFTESEKKFIKGTADFFALSFGPTLSFQLLDPHMKFRQLESPSLRQLLSWIDLEYNHPQIFIVENGWFVSGTTKRDDAKYMYYLKKFIMETLKAIKLDGVDVIGYTAWSLMDGFEWHRGYSIRRGLFYVDFLSQEKTLLPKSSALFYQKLIEKNGFPPLPENQPLEGTFPCDFAWGIVDNYIQVDTTLSQFTDLNVYLWDVHHSKRLIKVDGVVTKKRKSYCVDFAAIQPQITLLQEMHVTHFRFSLDWALILPLGNQSQVNHTILQYYRCMVSELVRVNITPVVALWQPVAPNQGLPRLLARQGAWENPYTALAFAEYARLCFQELGGHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRFAADQFEPKPSMKHYRKIIDSNGFPGPETLEKFCPEEFTMCTECSFFHTRKPLVAFIAFLFLAFIVSLSLIFYYSKKGRRRYK

SEQ ID NO: 61 - protein, 1012 aa, Trachypithecus francoisi
MPASAPPRRPRPPPPSLSLLLVLLALGGRCLRAEPGDGAQTWARFARPPAPEAAGLFQGTFPDGFLWAVGSAAYQTEGGWQQHGKGASIWDTFTHHPLAPPGDSRIANLPSGAPSPPQPATADVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPIFIDGDYPESMKNNLSSLLPDFTESEKKFIKGTADFFALSFGPTLSFQLLDPHMKFRQLESPSLRQLLSWIDLEYNHPQIFIVENGWFVSGTTKRDDAKYMYYLKKFIMETLKAIKLDGVDVIGYTAWSLMDGFEWHRGYSIRRGLFYVDFLSQEKTLLPKSSALFYQKLIEKNGFPPLPENQPLEGTFPCDFAWGIVDNYIQVDTTLSQFTDLNVYLWDVHHSKRLIKVDGVVTKKRKSYCVDFAAIQPQITLLQEMHVTHFRFSLDWALILPLGNQSQVNHTILQYYRCMVSELVRVNITPVVALWQPVAPNQGLPRLLARQGAWENPYTALAFAQYARLCFQELGRHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRFAADQFEPKPSMKHYRKIIDSNGFPGPETLEKFCPEEFTMCTECSFFHTRKPLVAFIAFLFLAFIVSLSLIFYYSKKGRRRYK

SEQ ID NO: 62 - protein, 1010 aa, Chlorocebus sabaeus
MPASAPPRRPRPPPPSLLLVLLALGGRRLRAEPGDGAQTWARFARPPAPEAAGLFQGTFPDGFLWAVGSAAYQTEGGWQQHGKGASIWDTFTHHPLAPPGDSRIANVPSGAPSPLQPATGDVTSDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYTTGRLAPGIRGSPRLGYLVAHNLLLAHAKVWHLYNTSFRPTQGGQVSIALSSHWISPRRMTDHSIKECQKSLDFVLGWFAKPIFIDGDYPESMKNNLSSLLPDFTESEKKFIKGTADFFALSFGPTLSFQLLDPHMKFRQLESPSLRQLLSWIDLEYNHPQIFIVENGWFVSGTTKRDDAKYMYYLKKFIMETLKAIKLDGVDVIGYTAWSLMDGFEWHRGYSIRRGLFYVDFLSQEKTLLPKSSALFYQKLIEKNGFPPLPENQPLEGTFPCDFAWGIVDNYIQVDTTLSQFTDLNVYLWDVHHSKRLIKVDGVVTKKRKSYCGDFAAIQPQITLLQEMHVTHFRFSLDWALILPLGNQSQVNHTILQYYRCMVSELVRVNITPVVALWQPVAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFFLPYFTEDEKKLIQGTFDFLALSHYTTILVDSEKEDPIKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRFAADQFEPKPSMKHYRKIIDSNGFPGPETLEKFCPEEFTVCTECSFFHTRKPLVAFIAFLFFAFIVSLSLIFYYSKKGRRRYK

SEQ ID NO: 63 - protein, 1188 aa, Papio anubis
MAGDGECRIQALGFLLHLEQMQPPRNNQISQIAEATRIESSGSTVYFSYRELCRTDSGQSLIGSSNVQPELLWASEWGKSESRCFPNRALLQGPAGSRAQVGASWSAPFLPDEAAPGLISEDARPAKRMNLSVPETSCAAPGIWEKQVPSPRPLRHLAPSTAAPSLGLPSSRLSPRCPGQPHRPPWLPPSRPLSLLLVLLALGGRRLRAEPGDGAQTWARFARPPAPEAAGLFQGTFPDGFLWAVGSAAYQTEGGWQQHGKGASIWDTFTHHPLAPPGDSRIANVPSGAPSPLQPATGDVASDSYNNVFRDTEALRELGVTHYRFSISWARVLPNGSAGVPNREGLRYYRRLLERLRELGVQPVVTLYHWDLPQRLQDAYGGWANRALADHFRDYAELCFRHFGGQVKYWITIDNPYVVAWHGYATGRLAPGIRGSPRLGYLVAHNLLLAHAKVWHLYNTSFRPTQGGQVSIALSSHWINPRRMTDHSIKECQKSLDFVLGWFAKPIFIDGDYPESMKNNLSSLLPDFTESEKKFIKGTADFFALSFGPTLSFQLLDPHMKFRQLESPSLRQLLSWIDLEYNHPQIFIVENGWFVSGTTKRDDAKYMYYLKKFIMETLKAIKLDGVDVIGYTAWSLMDGFEWHRGYSIRRGLFYVDFLSQEKTLLPKSSALFYQKLIEKNGFPPLPENQPLEGTFPCDFAWGIVDNYIQVDTTLSQFTDLNVYLWDVHHSKRLIKVDGVVTKKRKSYCVDFAAIQPQITLLQEMHITHFRFSLDWALILPLGNQSQVNHTILQYYRCMVSELVRVNITPVVALWQPVAPNQGLPRLLARQGAWENPYTALAFAEYARLCFQELGHHVKLWITMNEPYTRNMTYSAGHNLLKAHALAWHVYNEKFRHAQNGKISIALQADWIEPACPFSQKDKEVAERVLEFDIGWLAEPIFGSGDYPWVMRDWLNQRNNFLLPYFTEDEKKLIQGTFDFLALSHYTTILVDSEKEDPVKYNDYLEVQEMTDITWLNSPSQVAVVPWGLRKVLNWLKFKYGDLPMYIISNGIDDGLHAEDDQLRVYYMQNYINEALKAHILDGINLCGYFAYSFNDRTAPRFGLYRFAADQFEPKPSVKHYRKIIDSNGFPGPETLETFCPEESTVCTECSFFHTRKPLVAFIAFLFFAFIVSLSLIFYYSKKGRRRYK

(19) United States
(12) Patent Application Publication
(10) Pub. No.: US 2023/0346977 A1
(43) Pub. Date: Nov. 2, 2023

(54) TREATMENT OF NEUROMUSCULAR DISEASES VIA GENE THERAPY THAT EXPRESSES KLOTHO PROTEIN

(71) Applicants: Universitat Autònoma de Barcelona, Barcelona (ES); Consorcio Centro de Investigación Biomédica en Red, Madrid (ES); Institució Catalana de Recerca i Estudis Avançats, Barcelona (ES); Fundació Hospital Universitari Vall d’Hebron-Institut de Recerca, Barcelona (ES)

(72) Inventors: Assumpció Bosch Merino, Barcelona (ES); Sergi Verdés Franquesa, Lleida (ES); Mireia Herrando Grabulosa, Centelles (ES); Xavier Navarro Acebes, Barcelona (ES); Miguel Chillon Rodriguez, Barcelona (ES)

(73) Assignees: Universitat Autònoma de Barcelona, Barcelona (ES); Consorcio Centro de Investigación Biomédica en Red, Madrid (ES); Institució Catalana de Recerca i Estudis Avançats, Barcelona (ES); Fundació Hospital Universitari Vall d’Hebron-Institut de Recerca, Barcelona (ES)

(21) Appl. No.: 18/299,989

(22) Filed: Apr. 13, 2023

Related U.S. Application Data
(60) Provisional application No. 63/330,684, filed on Apr. 13, 2022.

(51) Int. Cl. A61K 48/00; C12N 9/24; A61P 21/00; C12N 15/86

(52) U.S. Cl. CPC A61K 48/0058; C12N 9/24; A61P 21/00; C12N 15/86; C12N 2750/14143; C12N 2830/48

(57) ABSTRACT

The present disclosure provides gene constructs containing a nucleic acid sequences encoding a mammalian s-KL, operatively linked to a muscle cell-specific promoter, for use in the treatment of motor impairment that may manifest, for example, in a neuromuscular disorder or disease, utilizing viral and non-viral vectors with muscle cell and motor neuron tropisms containing gene constructs containing a nucleic acid sequence encoding a mammalian s-KL, operatively linked to a promoter such as a muscle cell-specific promoter, is delivered in pharmaceutical compositions containing the expression vector, isolated cells containing the expression vector, and methods of treating motor impairment and motor neuron diseases.

### RELATED APPLICATIONS

This application claims the benefit of priority under 35 U.S.C. § 119(e) to U.S. Provisional Application No. 63/330,684, filed Apr. 13, 2022, which is incorporated herein by reference in its entirety.

### TECHNICAL FIELD

The present disclosure relates to the field of medicine, and more particularly to medical approaches for treating neuromuscular disorders or diseases characterized by motor impairment and motor neuron diseases such as amyotrophic lateral sclerosis.

### SEQUENCE LISTING

The present application contains a Sequence Listing which has been submitted electronically in ASCHII format and is hereby incorporated by reference in its entirety. The XML copy, created on Apr. 13, 2023, is named 58578-001NUS.xml and is 162 KB bytes in size.

### BACKGROUND

Motor impairment, according to the International Neuromodulation Society, involves the partial or total loss of function of a body part, usually a limb or limbs. Diseases causing motor impairment may result in muscle weakness, poor stamina (i.e., fatigue), lack of muscle control, or total paralysis. Motor impairment is a major cause of physical disability. It is broadly caused by peripheral problems affecting muscles, problems in the central nervous system affecting output to muscles, and sensory problems affecting muscles, movement and balance. Motor impairment is often evident in neuromuscular diseases, in neurological conditions, and motor neuron diseases but it can also result from cancers of the central and the peripheral nervous system, or even in traumatic injuries.

A neuromuscular disease is any disease affecting motor neurons in the spinal cord or central nervous system (CNS), the peripheral nervous system (PNS), the neuromuscular junction, or skeletal muscle, all of which are components of the motor unit, thus ultimately affecting the movement ability of the subject. Damage to motor neurons in the spinal cord, the CNS, the PNS, the neuromuscular junction, or the skeletal muscle can cause muscle atrophy and weakness. Issues with sensation can also occur. Neuromuscular diseases can be acquired or genetic. Mutations of more than 500 genes have shown to be causes of neuromuscular diseases. Other causes include nerve or muscle degeneration, autoimmunity, toxins, medications, malnutrition, metabolic derangements, hormone imbalances, infection, nerve compression/entrapment, comprised blood supply, and trauma.

Examples of neuromuscular diseases and disorders include Amyotrophic lateral sclerosis (ALS), Charcot-Marie-Tooth disease, Multiple sclerosis, Muscular dystrophy, Myasthenia gravis, Myopathy, Myositis, including polymyositis and dermatomyositis, Peripheral neuropathy, Neuromyotonia, Lambert-Eaton disease, Friedreich's ataxia, Spinal Muscular Atrophy (SMA), spinal cord injuries, peripheral nerve injuries, traumatic nerve injuries, and muscle metabolic diseases. Some of these neuromuscular diseases are further classified as motor neuron diseases or motor neuron diseases (MNDs), which are a group of rare neurodegenerative disorders that selectively affect motor neurons, the cells which control voluntary muscles of the body. Examples of MND include amyotrophic lateral sclerosis (ALS), progressive bulbar palsy (PBP), pseudobulbar palsy, progressive muscular atrophy (PMA), primary lateral sclerosis (PLS), spinal muscular atrophy (SMA), and monomelic amyotrophy (MMA), as well as some rarer variants resembling ALS.

Amyotrophic lateral sclerosis (ALS) is a fatal neurodegenerative disorder of the motor system and the most frequent form of motoneuron diseases (MND), with an incidence of 1-5 per 100,000 persons-year. Patients with ALS suffer from progressive weakness and paralysis of the muscles and usually die within 2-5 years from diagnosis, mostly because of respiratory failure. Any skeletal muscle can be affected, which causes multiple different clinical symptoms among patients. These vary from cramps to fasciculation, marked weakness and muscle atrophy, hyper-reflexia and spasticity, and in some instances mood and memory function are also altered. Sporadic ALS (sALS) accounts for most of the cases, with a peak age of onset around 60 years. About 5-10% of the cases are familial (fALS) caused by inherited mutations, most being autosomal dominant, and with an age of onset a decade earlier than for sporadic cases. Cases in patients younger than 25 years are designated as juvenile onset ALS (jALS) and usually have an autosomal recessive inheritance. Some of the most prevalent fALS genetic causes are mutations in Cu/Zn superoxide dismutase 1 (SOD1), TAR-DNA binding protein (TDP-43), fused in sarcoma (FUS) and hexanucleotide repeat expansions in chromosome 9 open reading frame 72 (C9orf72). The neuropathological hallmark of ALS is that it affects both upper and lower motoneurons (MNs), distinguishing it from other MND. Neurodegeneration occurs in the corticospinal and corticobulbar tracts, with loss of large pyramidal neurons in the primary motor cortex, and in the anterior horns of the brainstem and spinal cord.

Denervation of endplates and axonal retraction is thought to lead, in a “dying-back” pattern, to the death of MNs and subsequent muscle atrophy. A “dying-forward” process is also being debated, in which primary damage occurs in upper or lower MNs and degeneration extends anterogradely to descending axonal projections.

Unfortunately, as well as for other neuromuscular diseases, in particular motor neuron diseases, to date there is no effective treatment for ALS. Despite decades of intense research and clinical trials, most promising preclinical therapies have failed in translating to successful human trials. Riluzole (Riluzole®) and Edaravone (Radicava®) are the only drugs approved for the treatment of ALS. Both prolong survival merely by a few months and mildly improve motor function, so symptomatic and palliative measures (including feeding and respiratory support) are the mainstay of patients' management. Interestingly, most of the compounds studied interfere with a single mechanism involved in MNs death. Thus, one of the main difficulties in developing effective therapies for ALS rises on the multiple events that contribute to MNs death. Given the multifactorial mechanisms of ALS pathogenesis, the use of combinatorial or multi-target therapies that act simultaneously on several mechanisms and target different cell types might result in enhanced therapeutic outcomes and maximize translational effects.

Thus, in spite of the efforts made so far, there is still a need for efficient and safe treatments for motor impairments of different etiologies.

### SUMMARY OF THE DISCLOSURE

The present disclosure pertains to a novel therapy for the prevention and/or treatment of motor impairment in neuromuscular diseases and other movement disorders based on the administration of certain variants of the mammalian klotho protein, in particular an alternative RNA splicing variant of Klotho (s-KL). To date, no evidence exists that certain klotho variants could be safely administered to muscle cells (either in protein form or as gene therapy) to treat motor impairment.

In one aspect, the present disclosure provides a gene construct comprising a first promoter operatively linked to a nucleic acid sequence encoding a mammalian s-KL or a functional variant thereof, wherein the first promoter is muscle cell-specific promoter. In some embodiments, the s-KL amino acid sequence is human s-KL, e.g., SEQ ID NO:1. In some embodiments, s-KL amino acid sequence is mouse s-KL, e.g., SEQ ID NO: 3. Representative functional variants may have at least 85% sequence identity to SEQ ID NO:1 or SEQ ID NO: 3. In some embodiments, the promoter is a constitutive muscle cell-specific promoter. In some embodiments, the promoter is an inducible muscle cell-specific promoter. In some embodiments, the muscle cell-specific promoter is the human desmin promoter. In some embodiments, the nucleic acid sequence encoding a mammalian s-KL or a functional variant thereof is operatively linked to at least one additional promoter that is different from the first promoter, e.g., a second promoter. In some embodiments, the second promoter is a muscle cell-specific promoter. In some embodiments, the second promoter is a neuronal cell-specific promoter. In some embodiments, the second promoter is inducible. In some embodiments, the second promoter is constitutive. In some embodiments, the second promoter is ubiquitous. In some embodiments, the second promoter is a zinc-driven metallothionein promoter.

In another aspect, the present disclosure provides a plasmid comprising the gene construct comprising (e.g., having integrated or cloned therein) a first promoter operatively linked to a nucleic acid sequence encoding a mammalian s-KL or a functional variant thereof, wherein the first promoter is muscle cell-specific promoter, and an initiation sequence operatively linked to the first promoter. While capable of functioning as a vector in their own right, plasmids may be useful for the preparation of more complex expression vectors, for example, AAV and lentivirus vectors. The plasmids may also be useful for obtaining mRNA after linearization of the plasmid and an appropriate in vitro transcription, which can be further encapsulated or complexed, if desired, with cationic lipids.

In another aspect, the present disclosure provides an expression vector, comprising (e.g., having integrated or cloned therein) the gene construct or the plasmid containing (a) a muscle cell-specific first promoter operatively linked to a nucleic acid encoding a mammalian s-KL or functional variant thereof, wherein the expression vector may or may not have a muscle cell tropism; or (b) a first promoter functional in a muscle cell, a neuronal cell, or an induced pluripotent stem cell (iPSC) operatively linked to a nucleic acid sequence encoding a mammalian s-KL or functional variant thereof, wherein the expression vector has muscle cell tropism. In some embodiments, the promoter is a muscle cell-specific promoter, and the expression vector has a neuronal cell tropism that serves to preferentially target the vector to neuronal cells. In some embodiments, the vector is a viral expression vector such as a DNA-viral expression vector or an RNA-viral expression vector. In other embodiments, the expression vector is a non-viral expression vector. In some embodiments, the expression vector is an adeno-associated virus (AAV) vector of a serotype with muscle cell tropism; or which is an AAV vector is of a serotype with neuronal cell tropism.

In some embodiments, the s-KL polypeptide comprises or consists of SEQ ID NO: 1, which is the human s-KL, or a functional variant thereof. In some embodiments, the variant comprises or consists of a sequence having at least 85% sequence identity to SEQ ID NO: 1. In some embodiments, the polypeptide has a sequence of at least 90% identical to SEQ ID NO: 1. In some embodiments, the polypeptide has a sequence of at least 95% identity to SEQ ID NO: 1. In some embodiments, the polypeptide has a sequence of at least 98% identity to SEQ ID NO: 1. In some embodiments, the polypeptide has a sequence of at least 99% identity to SEQ ID NO: 1. In other embodiments, the polypeptide has a sequence of SEQ ID NO: 2, which is the murine s-KL.

In some embodiments the expression vector is a viral vector. In some embodiments, the viral vector is an adeno-associated virus of a serotype which in some embodiments, is any one of AAV1, AAV2, AAV4, AAV5, AAV6, AAV7, AAV8, AAV9, AAV9p31, AAVrh10, PHPeB, 9P31, AAVrh74, and AAVMyo.

In some embodiments, the AAV vector contains an adeno-associated virus capsid polypeptide that has the amino acid sequence of any one of SEQ ID NOs: 17-29. In some embodiments, the AAV vector contains an adeno-associated virus capsid polypeptide that has the amino acid sequence of SEQ ID NO: 17 or 26.

In some embodiments, the viral vector is an AAV vector is of a serotype with neuronal cell tropism, which in some embodiments is any one of AAV1, AAV8, or AAV9.

In some embodiments, the vector is a lipid-based vector. In some embodiments, the lipid-based vector is a lipid nanoparticle (LNP) or a liposome.

In another aspect, the disclosure provides a pharmaceutical composition comprising a therapeutically effective amount of a plasmid or an expression vector as defined herein and a pharmaceutically acceptable carrier.

In another aspect, the disclosure provides an isolated cell which contains a gene construct or a plasmid as disclosed herein, wherein the isolated cell is a human muscle cell, a human neuronal cell, or a human induced pluripotent stem cell (iPSC) (which may be induced ex vivo to differentiate into a muscle cell or a neuronal cell or may differentiate in vivo). In some embodiments, the isolated cell is a neuronal cell, e.g., a motor neuron. In some embodiments, the isolated cell is a striated muscle cell or a skeletal muscle cell.

Another aspect of the disclosure is a method of treating diseases or disorders characterized by motor impairment in a subject. In some embodiments, the neuromuscular disease is Amyotrophic lateral sclerosis (ALS). In some embodiments, the ALS is sporadic ALS (sALS) or familial ALS (fALS). In some embodiments, the neuromuscular disease is Multiple sclerosis. In some embodiments, the neuromuscular disease is Muscular dystrophy. In some embodiments, the neuromuscular disease is Spinal Muscular Atrophy (SMA). In some embodiments, the neuromuscular disease is spinal cord injuries, peripheral nerve injuries or traumatic nerve injuries. In some embodiments, the method may entail administering to the subject a pharmaceutical composition comprising a therapeutically effective amount of the plasmid or the expression vector containing the gene construct. In some embodiments, the method may entail continuous administration (e.g., infusion) of the composition to the subject. In some embodiments, the continuous infusion may be conducted over a period of about 1 hour to about 24 hours/days for one or more days. In some embodiments, the subject does not exhibit motor impairment symptoms.

Another aspect of the disclosure is a cell therapy comprising administering to a subject in need thereof an isolated cell as disclosed herein. This method may entail an ex vivo approach wherein muscle cells or neuronal cells are isolated from the subject and transformed or transfected with the gene construct, the plasmid, or the expression vector, and then administered to the subject. In some embodiments, the muscle cells or neuronal cells are enriched prior to or after transformation. In other embodiments, partially pluripotent or stem-cell like cells are transformed or transfected with the expression vector and induced to differentiate into a specific cell type (e.g., muscle cells or neurons) in vitro. Once integrated with the expression vector (which may occur prior to or subsequent to differentiation), the cells are administered to the subject. In some embodiments, the method is autologous in the sense that it may entail isolating terminally differentiated cells from a subject, reprogramming (i.e., inducing) pluripotency in those cells to generate iPSCs, integrating the expression vector into the iPSCs, causing differentiation of the iPSCs into neuronal or muscle cells, and administering the differentiated cells into the subject. In some embodiments, the cells are caused to differentiate into muscle cells. In some embodiments, the cells are caused to differentiate into neuronal cells. In some embodiments, the iPSCs cells are allowed to differentiate after administration to the subject.

Without intending to be bound by theory, the constructs, methods etc. disclosed herein may increase mammalian s-KL protein levels in skeletal muscle cells which protects the cells from toxic insults and preserves muscle cell functionality and/or motor neuron functionality or which improves motor impairment associated with diseases and disorders, such as ALS, that are characterized by motor impairment.

As shown in the working examples herein, the present inventors have found that the administration of a mammalian secreted RNA splicing variant of Klotho (s-KL) protein, in a mouse model of ALS disease significantly delayed the progression of the disease. Furthermore, administration of a mammalian s-KL protein in gene therapy with the AAVmyo muscle cell tropic expression vector in a mouse model of ALS disease delayed the progression of disease while reducing the amount of expression vector required compared to other AAV vectors.

The present disclosure may overcome one or more disadvantages associated with known therapies, such as treatment with Klotho protein. The relatively short half-life of the Klotho protein in vivo, about 7.5 hours, results in approximately a log of decrease every 24 hours. In contrast, treatment in accordance with the present disclosure may have an effect akin to administration of mammalian s-Klotho protein via continued infusion.

### BRIEF DESCRIPTION OF DRAWINGS

FIG. 1 shows the s-KL mRNA expression (fold-change in ALS mice compared to WT animals) illustrated as mean±SEM in the several studied tissues (n=7 SOD1, 3-10 WT mice per group, ***p<0.001, **p<0.01, *p<0.05).

FIG. 2A and FIG. 2B show the preserved neuronal survival located at the ventral horn (VH) of the spinal cord from glutamate-induced excitotoxicity (GLUT+) provoked by the induced overexpression of s-KL, compared to samples submitted to Glut+ excitotoxicity and transduced (or not) with a null vector. In FIG. 2A, a fluorescence microscopic image, and in FIG. 2B, a bar diagram, being the second bar of each set the one glutamate-induced excitotoxicity (GLUT+). All data as the mean±SEM (n=6 per group, ***p<0.001, **p<0.01, *p<0.05). SMI 32 are the motor neuron cells recognized by the SMI32 antibody.

FIG. 3 is a schematic view of a gene therapy strategy to systemically treat mice (SOD1 model) with a vector with muscle cell tropism and with a gene construct including a promoter specific for striated (skeletal) muscle cells.

FIG. 4A and FIG. 4B are two-line plots showing the Compound Muscle Action Potential (CMAP) values (amplitude in mV) of Plantar muscles (FIG. 4A) or of Tibialis anterior (FIG. 4B) for wild type (WT), SOD1 mock (AAV-null), SOD1 s-KL low dose (AAV-s-KL), SOD1 s-KL high dose (AAV-s-KL).

FIG. 5 is a bar diagram illustrating the Motor evoked potentials (MEP) recorded from Tibialis anterior (TA) muscles. The first bar of the set is for the assay in SOD1 mock, the second bar is for SOD1 s-KL low dose, and the third bar is the SOD1 s-KL high dose. (***p<0.001, **p<0.01, *p<0.05, relative to SOD1 mock, mean±SEM).

FIG. 6A and FIG. 6B are line plots showing the time (Time, s) to fall in a rotarod (FIG. 6A), and the grip strength (Force (g) in FIG. 6B) observed in the animals as disclosed in previous FIG. 4A-FIG. 5. (***p<0.001, **p<0.01, *p<0.05, SOD1 high dose relative to SOD1 mock, mean±SEM).

FIG. 7 displays the clinical disease onset of the animals as disclosed in previous FIG. 4A-FIG. 5.

FIG. 8A-FIG. 8C show neuromuscular junctions (NMJ), to see the occupancy of presynaptic terminals. In FIG. 8A, a fluorescence microscope image with Gastrocnemius longitudinal sections were labeled for neurofilament 200 (NF200, dark grey in image), anti-synaptophysin and alfa-bungarotoxin (BTX, light grey in image). In FIG. 8B, there are indicated the percentage of occupied endplates in WT mice, and in mock (SOD1 mock) and treated (SOD1s-KL). In FIG. 8C, there are indicated the muscle mass per body weight (mg/g) of WT mice, and in mock (SOD1 mock) and treated (SOD1s-KL). (***p<0.001, **p<0.01, *p<0.05, SOD1 mock vs. SOD1 high dose, data presented as mean±SEM).

FIG. 9A-FIG. 9B show the preserved motor neuron survival located in the lumbar spinal cord in SOD1 mice provoked by the induced overexpression of s-KL, compared to samples from wild type and SOD1 mice transduced with a null vector. FIG. 9A is a white light microscopic image, and FIG. 9B is a bar diagram that shows or quantifies the number of motor neurons shown in FIG. 9A. (all data as the mean±SEM (n=8 per group, ***p<0.001)).

FIG. 10A-FIG. 10D show the reduced microglia activity in the spinal cord in SOD1 mice provoked by the induced overexpression of s-KL, compared to samples from wild type and SOD1 mice transduced with a null vector. FIG. 10A is a fluorescence microscopic image, and FIG. 10B-FIG. 10D are a set of bar diagrams that show or quantify the number of Integrated Density (arbitrary units) of each of the images stained for Iba1, GFAP, and Vimentin (all data as the mean±SEM (n=8 per group, **p<0.01, *p<0.05)).

FIG. 11 is a schematic view of a gene therapy strategy to systemically treat mice (SOD1^{G93A} model) with a AAV vector with muscle cell tropism (AAVmyo) and with a gene construct including a promoter specific for striated (skeletal) muscle cells.

FIGS. 12A-FIG. 12B are a set of line plots showing the Compound Muscle Action Potential (CMAP) values (amplitude in mV) of Plantar muscles (FIG. 12A) and Tibialis anterior (FIG. 12B), for wild type (WT mock), SOD1 mock, SOD1 s-KL low dose (AAVmyo-AAV-s-KL), and SOD1 s-KL high dose (AAVmyo-AAV-s-KL).

FIG. 13 is a bar diagram illustrating the Motor evoked potentials (MEP) recorded from Tibialis anterior (TA) muscles. The first bar of the set is for the assay in SOD1 mock, the second bar is for SOD1 AAVmyo-s-KL low dose, and the third bar is the SOD1 AAVmyo-s-KL high dose (***p<0.001, **p<0.01, *p<0.05, relative to SOD1 mock, mean±SEM).

FIGS. 14A-FIG. 14B are line plots showing the time (Time, s) to fall in a rotarod (FIG. 14A), and the grip strength (Force (g) in FIG. 14B) observed in the animals as illustrated in FIGS. 12A-FIG. 13.

FIG. 15 displays the clinical disease onset of the animals illustrated in FIGS. 12A-FIG. 14B.

### DETAILED DESCRIPTION OF THE DISCLOSURE

### Definitions

All terms as used herein in this application, unless otherwise stated, shall be understood in their ordinary meaning as known in the art. Other more specific definitions for certain terms as used in the present application are as set forth below and are intended to apply uniformly through-out the specification and claims unless an otherwise expressly set out definition provides a broader definition.

As used herein, the indefinite articles “a” and “an” are synonymous with “at least one” or “one or more.” Unless indicated otherwise, definite articles used herein, such as “the” also include the plural of the noun.

Throughout the description and claims the word “comprise” and variations of thereof, are not intended to exclude other technical features, additives, components, elements or steps. Furthermore, the word “comprise(s)” encompasses the case of “consisting of”.

### Gene Constructs

In one aspect, the disclosure provides a gene construct (also referred to herein as an expression cassette), comprising a nucleic acid sequence operatively linked to a muscle cell-specific promoter. The terms “gene construct” and “expression cassette” are used herein synonymously and refer to a nucleic acid sequence that encodes a mammalian secreted RNA splicing variant of mammalian Klotho (s-KL) or a functional variant thereof, which is operatively linked to a muscle cell-specific promoter.

The term “nucleic acid,” also referred to herein as a “nucleic acid sequence,” as used herein refers to a polymer of nucleotides, each of which are organic molecules consisting of a nucleoside (a nucleobase and a five-carbon sugar) and a phosphate. The term nucleotide, unless specifically sated or obvious from context, includes nucleosides that have a ribose sugar (i.e., a ribonucleotide that forms ribonucleic acid, RNA) or a 2′-deoxyribose sugar (i.e., a deoxyribonucleotide that forms deoxyribonucleic acid, DNA). Nucleotides serve as the monomeric units of nucleic acid polymers or polynucleotides. The four nucleobases in DNA are guanine (G), adenine (A), cytosine (C) and thymine (T). The four nucleobases in RNA are guanine (G), adenine (A), cytosine (C) and uracil (U). Nucleic acids are linear chains of nucleotides (e.g., at least 3 nucleotides) chemically bonded by a series of ester linkages between the phosphoryl group of one nucleotide and the hydroxyl group of the sugar (i.e., ribose or 2′-deoxyribose) in the adjacent nucleotide.

The term “promoter” as used herein refers to a nucleic acid sequence that regulates, directly or indirectly, the transcription of a corresponding nucleic acid sequence to which it is operably linked, which in the context of the present disclosure, is a mammalian s-KL or a functional variant thereof. A promoter may function alone to regulate transcription, or it may act in concert with one or more other regulatory sequences (e.g., enhancers or silencers, or regulatory elements that may be present in the gene construct or the expression vector). Promoters are located near the transcription start sites of genes, on the same strand and upstream on the DNA (towards the 5′ region of the sense strand). Promoters typically range from about 100-1000 base pairs in length. A “constitutive promoter” is a promoter that is active in all circumstances in the cell, contrary to others that are regulated, becoming active in the cell only in response to specific stimuli, such as “Inducible promoters”.

As used herein, the term “muscle” embraces skeletal muscle which refers to the voluntarily controlled, striated muscle type that is attached to the skeleton, representative examples of which include the diaphragm, biceps, the triceps, the quadriceps, the tibialis interior, and the gastrocnemius muscle.

The terms “muscle cell-specific” and “muscle-specific” as used herein in the context of promoters, refer to the preferential, selective, or predominant expression of the mammalian s-KL (or functional variant thereof) in muscle cells or muscle tissue, as compared to other (i.e., non-muscle) cells and tissues. In some embodiments, at least 50%, more particularly at least 60%, at least 70%, at least 75%, at least 80%, at least 85%, at least 90%, at least 95%, at least 98%, at least 99% or 100% of the expression occurs within muscle cells or tissue. In some embodiments, “muscle cell-specific” refers to substantially no leakage of the expressed mammalian s-KL (or functional variant thereof) to other organs or tissue than muscle, such as lung, liver, brain, kidney and/or spleen.

The term “functional in a muscle cell”, as used herein in the context of promoters, refers to promoters that regulate directly or indirectly, the transcription of a mammalian s-KL nucleic acid to which it is operably linked; however, it is not necessarily muscle cell-specific.

The term “operatively linked” as used herein is to be understood that the nucleic acid sequence encoding s-KL or a functional variant thereof is spatially situated or disposed in the gene construct the expression vector relative to the promoter to drive the expression of the nucleic acid coding sequence.

Klotho (KL) is a protein mainly expressed in the kidney and the brain, and to a lesser extent in the skeletal muscle, lung, urinary bladder, testes, and ovaries. Klotho has two different isoforms, one codifying for a full-length transmembrane protein (mKL), of about 140 KDa, which can be processed into a soluble form (p-KL) of about 130 KDa. The other isoform codifies for a secreted but shorter isoform (s-KL), of about 63 KDa. The mKL contains the KL1 and KL2 domains that can be shed by α/β/γ-secretases to produce p-KL or further cleaved to free the p-KL1 and p-KL2 domains (about 70KDa each). The s-KL contains the KL1-like domain plus an extra tail (of 15 amino acids at its C-terminus in mouse s-KL and 16 amino acids at its C-terminus in human s-KL). Both isoforms, m-KL and s-KL, have different expression levels as well as spatio-temporal expression profiles, suggesting different roles.

Soluble and secreted klotho are forms present in the circulation (e.g., blood) and cerebral spinal fluid (CSF) and function as circulating hormones, exerting biological actions on distant organs and multiple systems by binding to cell-surface receptors and repressing signals, such as insulin and insulin-like growth factor 1 (IGF1) signals. It must be noted that although a similar abbreviation (s-KL) is sometimes used in the prior art to name soluble Klotho (which is the processed version of transmembrane Klotho) and secreted Klotho (which is the RNA splicing variant), these two variants present a different structure.

The term “secreted RNA splicing variant of mammalian Klotho” abbreviated herein as “s-KL”, refers to the protein resulting from alternative RNA splicing of the transcript encoding full-length transmembrane form of Klotho protein (m-KL), which generates a truncated form of the protein (i.e., s-KL) that is formed by the KL1-like domain, with an approximate weight of about 63 kDa, together with a specific secretion signal consisting of a 15 amino acid tail in mice, and a 16 amino acid tail in humans, that is not found in the m-KL transcript, and for this reason is also called the secreted isoform of klotho, s-KL, or the secreted RNA splicing variant of klotho protein. S-KL is different from other forms of soluble klotho, namely p-KL, p-KL1 and p-KL2. In this disclosure, m-KL refers to the full-length transmembrane form; p-KL stands for the soluble proteolyzed klotho (i.e., KL1-KL2), which is generated by cleavage of the m-KL and has a molecular weight of approximately 130 kDa; and p-KL1 and p-KL2 refer to the soluble klotho forms consisting of the KL1 domain and the KL2 domain of p-KL, respectively. m-KL refers to the full-length transcript encoding a single pass transmembrane protein with a molecular weight of approximately 140 kDa (m-KL). The protein contains three domains, including a short transmembrane domain at the C-terminus, an extracellular domain composed of two internal repeated sequences of about 550 amino acids called KL1 and KL2 respectively, and a very short intracellular domain of 10 amino acids. The extracellular domain of the transmembrane form can be cleaved by metalloproteinases ADAM10 and ADAM17 resulting in another form of soluble Klotho of about 130 kDa (abbreviated p-KL for proteolyzed membrane isoform). Moreover, there is a second recognition site for the proteases ADAM10 and 17 located between the KL1 and KL2 domains, which generates two new 70 kDa proteins, one containing the KL1 domain only, and the other one containing the KL2 domain. s-KL and its functional variants, do not embrace full-length m-KL or p-KL (containing both KL1 and KL2 domains). The mammalian s-KL polypeptides and functional variants thereof, encoded by the nucleic acid described herein, exclude the full-length m-KL and p-KL (containing both KL1-KL2 domains) forms of Klotho, each with a molecular weight of approximately 130 kDa.

SEQ ID NO: 1 is the amino acid sequence of human s-KL, which as described above, is the transcript from alternative splicing of α-klotho human gene, comprising the KL1 domain, with an approximate weight of about 63 kDa and a specific secretion signal consisting of a 16-amino acid tail that is not found in the m-KL or KL1 transcript. α-klotho human gene is located on Chromosome 13 NC_000013.11 (33016063 . . . 33066145) of the assembly GRCh38 (24 Dec. 2013) for the human genome maintained by the Genome Reference Consortium. SEQ ID NO: 1 is derived from the corresponding cDNA of SEQ ID NO: 3, which is turn is derived from the alternative splicing transcript of the mRNA having the GenBank database accession number NM_004795 of 5012 base pairs, version 3 of 3 May 2014 (the sequence of which is hereby incorporated by reference).

The amino acid sequence of human s-KL is as follows (SEQ ID NO: 1):

| | |
|---|---|
| mpasapprrp rppppslsll lvllglggrr lraepgdgaq twarvsrppa peaaglfqgt | 60 |
| fpdgflwavg saayqteggw qqhgkgasiw dtfthhplap pgdsrnaslp lgapsplqpa | 120 |
| tgdvasdsyn nvfrdtealr elgvthyrfs iswarvlpng sagvpnregl ryyrrllerl | 180 |
| relgvqpvvt lyhwdlpqrl qdayggwanr aladhfrdya elcfrhfggq vkywitidnp | 240 |
| yvvawhgyat grlapgirgs prlgylvahn lllahakvwh lyntsfrptq ggqvsialss | 300 |
| hwinprrmtd hsikecqksl dfvlgwfakp vfidgdypes mknnlssilp dftesekkfi | 360 |
| kgtadffalc fgptlsfqll dphmkfrqle spnlrqllsw idlefnhpqi fivengwfvs | 420 |
| gttkrddaky myylkkfime tikaikldgv dvigytawsl mdgfewhrgy sirrglfyvd | 480 |
| flsqdkmllp kssalfyqkl iekngfpplp enqplegtfp cdfawgvvdn yiqvsqltkp | 540 |
| issltkpyh | |

The nucleic acid sequence of a representative complementary DNA (cDNA) encoding human s-KL (SEQ ID NO: 1) is set forth below and identified as SEQ ID NO: 3 (s-KL specific secretion sequence boxed below):

| | |
|---|---|
| atgcccgcca gcgccccgcc gcgccgcccg cgtccaccgc cgccgtcgct gtcgctgctg | 60 |
| ctggtgctgc tgggcctggg cggacgacgc ctgcgtgcgg agccgggcga cggcgcgcag | 120 |
| acctgggccc gtgtctcgcg gcctcctgcc cccgaggccg cgggcctctt ccagggcacc | 180 |
| ttccccgacg gcttcctctg ggccgtgggc agcgccgcct accagaccga gggcggctgg | 240 |
| cagcagcacg gcaagggtgc gtccatctgg gacacgttca cccaccaccc cctggcaccc | 300 |
| ccgggagact cccggaacgc cagtctgccg ttgggcgccc cgtcgccgct gcagcccgcc | 360 |
| accggggacg tagccagcga cagctacaac aacgtcttcc gcgacacgga ggcgctgcgc | 420 |
| gagctcgggg tcactcacta ccgcttctcc atctcgtggg cgcgagtgct ccccaatggc | 480 |
| agcgcgggcg tccccaaccg cgaggggctg cgctactacc ggcgcctgct ggagcggctg | 540 |
| cgggagctgg gcgtgcagcc cgtggtcacc ctgtaccact gggacctgcc ccagcgcctg | 600 |
| caggacgcct acggcggctg ggccaaccgc gccctggccg accacttcag ggattacgcg | 660 |
| gagctctgct tccgccactt cggcggtcag gtcaagtact ggatcaccat cgacaacccc | 720 |
| tacgtggtgg cctggcacgg ctacgccacc gggcgcctgg cccccggcat ccggggcagc | 780 |
| ccgcggctcg ggtacctggt ggcgcacaac ctcctcctgg ctcatgccaa agtctggcat | 840 |
| ctctacaata cttctttccg tcccactcag ggaggtcagg tgtccattgc cctaagctct | 900 |
| cactggatca atcctcgaag aatgaccgac cacagcatca aagaatgtca aaaatctctg | 960 |
| gactttgtac taggttggtt tgccaaaccc gtatttattg atggtgacta tcccgagagc | 1020 |
| atgaagaata acctttcatc tattctgcct gattttactg aatctgagaa aaagttcatc | 1080 |
| aaaggaactg ctgacttttt tgctctttgc tttggaccca ccttgagttt tcaacttttg | 1140 |
| gaccctcaca tgaagttccg ccaattggaa tctcccaacc tgaggcaact gctttcctgg | 1200 |
| attgaccttg aatttaacca tcctcaaata tttattgtgg aaaatggctg gtttgtctca | 1260 |
| gggaccacca agagagatga tgccaaatat atgtattacc tcaaaaagtt catcatggaa | 1320 |
| accttaaaag ccatcaagct ggatggggtg gatgtcatcg ggtataccgc atggtccctc | 1380 |
| atggatggtt tcgagtggca cagaggttac agcatcaggc gtggactctt ctatgttgac | 1440 |
| tttctaagcc aggacaagat gttgttgcca aagtcttcag ccttgttcta ccaaaagctg | 1500 |
| atagagaaaa atggcttccc tcctttacct gaaaatcagc ccctagaagg gacatttccc | 1560 |
| 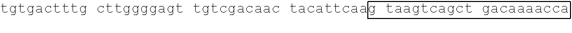 | 1620 |
| 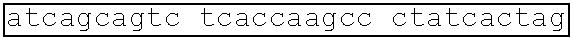 | 1650 |

Rodent s-KL's may also be useful in the present disclosure. SEQ ID NO: 2 is the amino acid sequence of the transcript from alternative splicing of α-klotho mouse gene, comprising the KL1 domain sequence, with an approximate weight of 70 kDa with a specific secretion signal consisting of 15 amino acid tail that is not found in the m-KL transcript. α-klotho mouse gene is the one located in Chromosome 5 (150,952,607-150,993,809) of UCSC Genome Browser on Mouse July 2007 (NCBI37/mm9) Assembly for the mouse genome. SEQ ID NO: 2 derived from the corresponding cDNA of SEQ ID NO: 4, which in turn was derived from the alternative splicing transcript of the mRNA sequence with the GenBank database accession number NM_013823 of 5124 base pairs, version 2 of 15 Feb. 2015 (the sequence of which is hereby incorporated by reference).

The amino acid sequence of mouse s-KL (SEQ ID NO: 2), is set forth below with:

| | |
|---|---|
| mlarapprrp prlvllrlll lhllllalra rclsaepgqg aqtwarfara papeaagllh | 60 |
| dtfpdgflwa vgsaayqteg gwrqhgkgas iwdtfthhsg aapsdspivv apsgapsppl | 120 |
| sstgdvasds ynnvyrdteg lrelgvthyr fsiswarvlp ngtagtpnre glryyrrlle | 180 |
| rlrelgvqpv vtlyhwdlpq rlqdtyggwa nraladhfrd yaelcfrhfg gqvkywitid | 240 |
| npyvvawhgy atgrlapgvr gssrlgylva hnlllahakv whlyntsfrp tqggrvsial | 300 |
| sshwinprrm tdynirecqk sldfvlgwfa kpifidgdyp esmknnlssl lpdftesekr | 360 |
| lirgtadffa lsfgptlsfq lldpnmkfrq lespnlrqll swidleynhp pifivengwf | 420 |
| vsgttkrdda kymyylkkfi metlkairld gvdvigytaw slmdgfewhr gysirrglfy | 480 |
| vdflsqdkel lpkssalfyq kliedngfpp lpenqplegt fpcdfawgvv dnyvqlsplt | 540 |
| kpsvglllph | |

The nucleic acid sequence of a representative complementary DNA (cDNA) nucleic acid sequence of mouse s-KL is set forth below with (SEQ ID NO: 4):

| | |
|---|---|
| atgctcgccc gcgcccctcc tcgccgcccg ccgcggctgg tgctgctccg tttgctgttg | 60 |
| ctgcatctgc tgctgctcgc cctgcgcgcc cgctgcctga gcgctgagcc gggtcagggc | 120 |
| gcgcagacct gggctcgctt cgcgcgcgct cctgccccag aggccgctgg cctcctccac | 180 |
| gacaccttcc ccgacggttt cctctgggcg gtaggcagcg ccgcctatca gaccgagggc | 240 |
| ggctggcgac agcacggcaa aggcgcgtcc atctgggaca ctttcaccca tcactctggg | 300 |
| gcggccccgt ccgactcccc gatcgtcgtg gcgccgtcgg gtgccccgtc gcctcccctg | 360 |
| tcctccactg gagatgtggc cagcgatagt tacaacaacg tctaccgcga cacagagggg | 420 |
| ctgcgcgaac tgggggtcac ccactaccgc ttctccatat cgtgggcgcg ggtgctcccc | 480 |
| aatggcaccg cgggcactcc caaccgcgag gggctgcgct actaccggcg gctgctggag | 540 |
| cggctgcggg agctgggcgt gcagccggtg gttaccctgt accattggga cctgccacag | 600 |
| cgcctgcagg acacctatgg cggatgggcc aatcgcgccc tggccgacca tttcagggat | 660 |
| tatgccgagc tctgcttccg ccacttcggt ggtcaggtca agtactggat caccattgac | 720 |
| aacccctacg tggtggcctg gcacgggtat gccaccgggc gcctggcccc gggcgtgagg | 780 |
| ggcagctcca ggctcgggta cctggttgcc cacaacctac ttttggctca tcccaaagtc | 840 |
| tggcatctct acaacacctc tttccgcccc acacagggag gccgggtgtc tatcgcctta | 900 |
| agctcccatt ggatcaatcc tcgaagaatg actgactata atatcagaga atgccagaag | 960 |
| tctcttgact ttgtgctagg ctggtttgcc aaacccatat ttattgatgg cgactaccca | 1020 |
| gagagtatga agaacaacct ctcgtctctt ctgcctgatt ttactgaatc tgagaagagg | 1080 |
| ctcatcagag gaactgctga cttttttgct ctctccttcg gaccaacctt gagctttcag | 1140 |
| ctattggacc ctaacatgaa gttccgccaa ttggagtctc ccaacctgag gcagcttctg | 1200 |
| tcttggatag atctggaata taaccaccct ccaatattta ttgtggaaaa tggctggttt | 1260 |
| gtctcgggaa ccaccaaaag ggatgatgcc aaatatatgt attatctcaa gaagttcata | 1320 |
| atggaaacct taaaagcaat cagactggat ggggtcgacg tcattgggta caccgcgtgg | 1380 |
| tcgctcatgg acggtttcga gtggcatagg ggctacagca tccggcgagg actcttctac | 1440 |
| gttgactttc tgagtcagga caaggagctg ttgccaaagt cttcggcctt gttctaccaa | 1500 |
| aagctgatag aggacaatgg ctttcctcct ttacctgaaa accagcccct tgaagggaca | 1560 |
| tttccctgtg actttgcttg gggagttgtt gacaactacg ttcagctgag tcctttgaca | 1620 |
| aaacccagtg tcggcctctt gcttcctcac taa | 1653 |

Additional mammalian s-KLs may be suitable for use in the present disclosure. Mammalian klotho genes containing the KL1 and KL2 domains are known in the art and/or may be readily identified in accordance with standard techniques. If a s-KL isoform from a given mammalian species has not been identified, a s-KL isoform may be readily derived from an identified mammalian klotho gene, specifically from the KL1 and/or KL2 domains with an appropriate C-terminal tail, in accordance with standard techniques.

Amino acid sequences of additional mammalian klotho genes native to the mammalian species Pan paniscus (pygmy chimpanzee, Accession No.: XP_034792458.1), Pan troglodytes (chimpanzee, Accession No.: XP_522655.2), Gorilla gorilla (western lowland gorilla, Accession No.: XP_030857339.1), Macaca fascicularis (crab-eating macaque, Accession No.: AAC77917.1, Q8WP17.1, XP 005586019.2, XP_005586019.3), Pongo abelii (Sumatran orangutan, Accession No.: XP 024086695.2, PNJ48590.1), Pongo pygmaeus (Bornean orangutan, Accession No.: XP_054302967.1), Piliocolobus tephrosceles (Ugandan red Colobus, Accession No.: XP_023064573.2), Nomascus leucogenys (northern white-cheeked gibbon, Accession No.: XP_030674267.1), Macaca thibetana (Tibetan macaqu, Accession No.: XP 050621876.1), Macaca nemestrina (pig-tailed macaque, Accession No.: XP_011746941.1), Macaca mulatta (Rhesus monkey, Accession No.: EHH28941.1, XP_001101127.2), Hylobates moloch (silvery gibbon, Accession No.: XP_032005041.1), Theropithecus gelada (gelada, Accession No.: XP_025220255.1), Rhinopithecus roxellana (golden snub-nosed monkey, Accession No.: XP_030778249.1), Trachypithecus francoisi (Francois's langur, Accession No.: XP_033091442.1), Chlorocebus sabaeus (green monkey, Accession No.: XP_007958284.1), and Papio anubis (olive baboon, Accession No.: XP_031511616.1) are disclosed in the accompanying sequence listing as SEQ ID NOs: 42-63 (the sequences of which are hereby incorporated by reference). Each mammalian klotho has at least 85% amino acid sequence identity to SEQ ID NO: 1 and SEQ ID NO: 2.

The present disclosure embraces functional variants of mammalian s-KL and use thereof in the disclosed compositions and methods. The functional variants may be non-naturally occurring.

Protein variants are well understood to those of skill in the art and can involve amino acid modifications that typically fall into one or more of three classes: substitutional, insertional, or deletional variants. In the present disclosure, the variants are “functional” in the sense that they are therapeutically effective in treatment of motor impairment that may occur, for example, in various diseases or disorders such as neuromuscular diseases and disorders.

The term “substitutional variant” as used herein when referred to polypeptides, refers to at least one amino acid in a native or starting sequence of a polypeptide is removed and a different amino acid is inserted in its place. The substitutions may be single, where only one amino acid in the polypeptide molecule is substituted, or they may be multiple, where two or more amino acids are substituted in the same polypeptide molecule.

The term “conservative substitution” as used herein when referred to polypeptides, refers to at least one amino acid in a native or starting sequence of a polypeptide is substituted with a different amino acid of similar size, charge, or polarity. Examples of conservative substitutions include the substitution of a non-polar (hydrophobic) residue for another non-polar residue, for example exchanging isoleucines, valines or leucines. Likewise, examples of conservative substitutions include the substitution of one polar (hydrophilic) residue for another, for example exchanging between arginine and lysine, between glutamine and asparagine, and between glycine and serine. Additional examples of conservative substitution include that of a basic amino acid such as lysine, arginine or histidine for another, or the substitution of one acidic residue such as aspartic acid or glutamic acid for another acidic residue.

The term “insertional variants” as used herein when referring to polypeptides, refers to variants with one or more amino acids inserted immediately adjacent to an amino acid at a particular position in a native or starting sequence. The term “immediately adjacent” to an amino acid as used herein refers to an amino acid connection through either the alpha-carboxy or alpha-amino functional group of the amino acid.

The term “deletional variants” as used herein when referring to polypeptides, refers to variants with one or more amino acids in the native or starting amino acid removed. Ordinarily, deletional variants will have one or more amino acids deleted in a particular region of the molecule.

In the present disclosure the term “identity” refers to the percentage of residues that are identical in the two sequences when the sequences are optimally aligned. If, in the optimal alignment, a position in a first sequence is occupied by the same amino acid as the corresponding position in the second sequence, the sequences exhibit identity with respect to that position. The percentage of identity determines the number of identical residues over a defined length in a given alignment. Thus, the level of identity between two sequences or (“percent sequence identity”) is measured as a ratio of the number of identical positions shared by the sequences with respect to the number of positions compared (i.e., percent sequence identity=(number of identical positions/total number of positions compared)×100). A gap, i.e., a position in an alignment where a residue is present in one sequence but not in the other, is regarded as a position with non-identical residues and is counted as a compared position.

As an illustration, by a polypeptide having an amino acid sequence having at least, for example, 95% identity to a reference amino acid sequence of SEQ ID NO:1 is intended that the amino acid sequence of the polypeptide is identical to the reference sequence except that the polypeptide sequence may include up to five amino acid alterations per each 100 amino acids of the reference amino acid of SEQ ID NO: 1. In other words, to obtain a polypeptide having an amino acid sequence of at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

A number of mathematical algorithms for rapidly obtaining the optimal alignment and calculating identity between two or more sequences are known and incorporated into a number of available software programs. For purposes of the present disclosure, the sequence identity between two amino acid sequences is preferably determined using algorithms based on global alignment, such as the Needleman-Wunsch algorithm (Needleman and Wunsch, J. Mol. Biol. 48:443-453 (1970), preferably implemented in the Needle program of the EMBOSS package (Rice et al., Trends Genet. 16 (6):276-277 (2000)); or the BLAST Global Alignment tool (Altschul et al., J. Mol. Biol. 215 (3):403-410 (1990)), using default settings. Local alignment also can be used when the sequences being compared are substantially the same length.

Representative functional variants mammalian s-KL, which may be naturally occurring or non-naturally occurring, have at least 85% amino acid sequence identity to SEQ ID NO: 1 and/or SEQ ID NO: 2. Representative functional s-KL variants may thus have 85%, 86%, 87%, 88%, 88.5%, 89%, 89.5%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, or 99.5% sequence identity or higher, to SEQ ID NO: 1 or SEQ ID NO:2. One representative functional variant of mammalian s-KL that has at least 85% amino acid sequence identity to SEQ ID NO: 1 is the KL1 domain (e.g., the cleavage product p-KL1 of p-KL).

In some embodiments, the polypeptide has an amino acid sequence having an amino acid sequence of at least 88% or 98% identity to SEQ ID NO: 1. In some embodiments, the polypeptide has an amino acid sequence having an amino acid sequence of at least 88% or 98% identity to SEQ ID NO: 2. In some embodiments, the polypeptide is a variant with amino acid insertions, for example, the in one embodiment, the last 15 amino acids of SEQ ID NO: 1 may be removed and replaced with

(SEQ ID NO: 17)
SQLTASVSSPPTRALSLASSAFLLGWRSWRILCPEPTR

The polypeptides with a percentage of identity of at least 88% with any of SEQ ID NO: 1 or SEQ ID NO: 2 encompass mammalian s-KLs other than human and mouse s-KL.

In another embodiment, optionally in combination with any one of the embodiments provided below, the mammalian s-KL polypeptide or functional variant thereof has a length equal to or lower than 645 amino acids, 600 amino acids, or 550 amino acids. In some embodiments, the polypeptide has the amino acid sequence of SEQ ID NO: 1 or a functional variant thereof having at least 85% sequence identity to SEQ ID NO: 1 and has a length equal to or lower than 645 amino acids, 600 amino acids, or 550 amino acids. In some embodiments, the polypeptide has the amino acid sequence of SEQ ID NO: 1 or a functional variant thereof having at least 85% identity to SEQ ID NO: 1, and wherein the variant has a length of 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 584, 586, 587, 588, 589, 590, 591, 592, 593, 594, 595, 596, 597, 598, 599, or 600 amino acids, or a length from 545 to 600 amino acids.

In some embodiment, optionally in combination with any of the embodiments provided above and below, the s-KL polypeptide encoded by the nucleic acid sequence is connected to a heterologous moiety by means of a peptide linkage.

As used herein, “heterologous moiety” refers to any molecule coupled to the polypeptide via a covalent peptide bond. In a particular embodiment, the heterologous moiety is located in either the N-terminal or the C-terminal end of the polypeptide. In a particular embodiment, the heterologous moiety is located in both the N-terminal and the C-terminal ends of the polypeptide.

The heterologous moiety can be, for example, a molecule that facilitates the purification of the polypeptide. In a particular embodiment, the heterologous moiety is a peptide. In an even more particular embodiment, the heterologous moiety is a polyhistidine track. As the skill in the art would understand, small peptides that assist in the purification of the protein can be maintained in the final compound without affecting its functionality.

In some embodiments, the heterologous moiety is a vehiculization agent. As known in the art, these agents facilitate absorption, transport and delivery of the polypeptide. Representative examples of vehiculization agents include dipalmitoyl-phosphatidyl-choline (DPCC) liposomes, micellular emulsions, dimethyformamide (DMF), and halogenated phentiazine.

A promoter that is operatively linked to the nucleic acid encoding mammalian s-KL or a functional variant thereof is a muscle-cell specific promoter. Representative examples of muscle-cell specific promoters that may be suitable for use in the present disclosure include a mammalian desmin (DES, also known as CSM1 or CSM2) promoter, the alpha 2 actinin (ACTN2, also known as CMD1AA) promoter, the filamin-C (FLNC, also known as actin-binding-like protein (ABLP), filamin-2 (FLN2), ABP-280, ABP280A, ABPA, ABPL, MFM5 or MPD4) promoter, the sarcoplasmic/endoplasmic reticulum calcium ATPase 1 (ATP2A1, also known as ATP2A or SERCA1) promoter, the troponin I type 1 (TNNI1, also known as SSTNI or 25TTNI) promoter, the myosin-1 (MYH1) promoter, the phosphorylatable, fast skeletal muscle myosin light chain (MYLPF) promoter, myosin 1 (MYH1, also known as MYHSA1, MYHa, MyC-2X/D or MyHC-2x) promoter, the alpha-3 chain tropomyosin (TPM3, also known as CFTD, NEM1, OK/Sc1.5, TM-5, TM3, TM30, TM30 nm, TM5, TPMsk3, TRK, h TM5 or hscp30) promoter, the ankyrin repeat domain-containing protein 2 (ANKRD2, also known as ARPP) promoter, the myosin heavy-chain (MHC) promoter, the myosin light-chain (MLC) promoter, the muscle creatine kinase (MCK) promoter, synthetic muscle promoters as described in Li et al., Nat. Biotechnol. 17 (3):241-245 (1999), such as the SPc5-12 promoter, the muscle creatine kinase (MCK) promoter, the dMCK promoter, the tMCK promoter consisting of respectively, a double or triple tandem of the MCK enhancer to the MCK basal promoter, as described in Wang et al., Gene Ther. 15 (22):1489-1499 (2008).

Hybrid (synthetic) muscle-cell specific promoters may also be useful. Types of such promoters may include muscle-specific enhancers and/or transcription factor binding sites, including combinations with other promoter elements from viruses or human sequences (e.g., CMV, CAG, or PGK). Representative examples of hybrid promoters include the hybrid alpha-myosin heavy chain enhancer/MCK enhancer (MHCK7; 770 bp); the MCK-C5-12 promoter as described in Wang et al., Gene Ther. 15 (22):1489-1499 (2008) and the cardiac and skeletal muscle-specific myosin chaperone Unc45b (195 bp) promoter as described in Rudeck et al., Genesis 54 (8):431-438 (2016). In some embodiments, the promoter is a mammalian muscle-cell promoter such as a human or murine desmin promoter, examples of which are described, for example, in U.S. Patent Application Publication 2020/00407746.

In some embodiments, the promoter is that which is natively associated with the gene encoding the human desmin protein, for which the promoter sequence is set forth below as SEQ ID NO: 5:

| | |
|---|---|
| gcggccgcac ccatgcctcc tcaggtaccc cctgcccccc acagctcctc tcctgtgcct | 60 |
| tctttcccag ccatgcgttc tcctctataa atacccgctc tggtatttgg ggttggcagc | 120 |
| tgttgctgcc agggagatgg ttgggttgac atgcggctcc tgacaaaaca caaacccctg | 180 |
| gtgtgtgtgg gcgtgggtgg tgtgagtagg gggatgaatc agggaggggg cgggggaccc | 240 |
| agggggcagg agccacacaa agtctgtgcg ggggtgggag cgcacatagc aattggaaac | 300 |
| tgaaagctta tcagaccctt tctggaaatc agcccactgt ttataaactt gaggccccac | 360 |
| cctcgagata accagggctg aaagaggccc gcctgggggc tgcagacatg cttgctgcct | 420 |
| gccctggcga aggattggca ggcttgcccg tcacaggacc cccgctggct gactcagggg | 480 |
| cgcaggcctc ttgcggggga gctggcctcc ccgcccccac ggccacgggc cgccctttcc | 540 |
| tggcaggaca gcgggatctt gcagctgtca ggggagggga ggcgggggct gatgtcagga | 600 |
| gggatacaaa tagtgccgac ggctgggggc cctgtctccc ctcgccgcat ccactctccg | 660 |
| gccggccgcc tgcccgccgc ctcctccgtg cgcccgccag cctcgcccgc gccgtcaccg | 720 |
| gttcgaacag gtaagcgccc ctaaaatccc tttggcacaa tgtgtcctga ggggagaggc | 780 |
| agcgacctgt agatgggacg ggggcactaa ccctcagggt ttggggttct gaatgtgagt | 840 |
| atcgccatct aagcccagta tttggccaat ctcagaaagc tcctggctcc ctggaggatg | 900 |
| gagagagaaa aacaaacagc tcctggagca gggagagtgt tcgcctcttg ctctccggct | 960 |
| ccctctgttg ccctctggtt tctccccagg ttcgaaggat cc | 1002 |

Therefore, in some embodiments, an inventive gene construct may include the human desmin promoter of SEQ ID NO: 5 operably linked to the mouse s-KL of SEQ ID NO: 2, and has a nucleic acid sequence as follows (SEQ ID NO: 6):

| | |
|---|---|
| gcggccgcac ccatgcctcc tcaggtaccc cctgcccccc acagctcctc tcctgtgcct | 60 |
| tctttcccag ccatgcgttc tcctctataa atacccgctc tggtatttgg ggttggcagc | 120 |
| tgttgctgcc agggagatgg ttgggttgac atgcggctcc tgacaaaaca caaacccctg | 180 |
| gtgtgtgtgg gcgtgggtgg tgtgagtagg gggatgaatc agggaggggg cgggggaccc | 240 |
| agggggcagg agccacacaa agtctgtgcg ggggtgggag cgcacatagc aattggaaac | 300 |
| tgaaagctta tcagaccctt tctggaaatc agcccactgt ttataaactt gaggccccac | 360 |
| cctcgagata accagggctg aaagaggccc gcctgggggc tgcagacatg cttgctgcct | 420 |
| gccctggcga aggattggca ggcttgcccg tcacaggacc cccgctggct gactcagggg | 480 |
| cgcaggcctc ttgcggggga gctggcctcc ccgcccccac ggccacgggc cgccctttcc | 540 |
| tggcaggaca gcgggatctt gcagctgtca ggggagggga ggcgggggct gatgtcagga | 600 |
| gggatacaaa tagtgccgac ggctgggggc cctgtctccc ctcgccgcat ccactctccg | 660 |
| gccggccgcc tgcccgccgc ctcctccgtg cgcccgccag cctcgcccgc gccgtcaccg | 720 |
| gttcgaacag gtaagcgccc ctaaaatccc tttggcacaa tgtgtcctga ggggagaggc | 780 |
| agcgacctgt agatgggacg ggggcactaa ccctcagggt ttggggttct gaatgtgagt | 840 |
| atcgccatct aagcccagta tttggccaat ctcagaaagc tcctggctcc ctggaggatg | 900 |
| gagagagaaa aacaaacagc tcctggagca gggagagtgt tggcctcttg ctctccggct | 960 |
| ccctctgttg ccctctggtt tctccccagg ttcgaaggat ccactagtcc agtgtggtgg | 1020 |
| aattcgccgc caccatgcta gcccgcgccc ctcctcgccg cccgccgcgg ctggtgctgc | 1080 |
| tccgtttgct gttgctgcat ctgctgctgc tcgccctgcg cgcccgctgc ctgagcgctg | 1140 |
| agccgggtca gggcgcgcag acctgggctc gcttcgcgcg cgctcctgcc ccagaggccg | 1200 |
| ctggcctcct ccacgacacc ttccccgacg gtttcctctg ggcggtaggc agcgccgcct | 1260 |
| atcagaccga gggcggctgg cgacagcacg gcaaaggcgc gtccatctgg gacactttca | 1320 |
| cccatcactc tggggcggcc ccgtccgact ccccgatcgt cgtggcgccg tcgggtgccc | 1380 |
| cgtcgcctcc cctgtcctcc actggagatg tcgccagcga tagttacaac aacgtctacc | 1440 |
| gcgacacaga ggggctgcgc gaactggggg tcacccacta ccgcttctcc atatcgtggg | 1500 |
| cgcgggtgct ccccaatggc accgcgggca ctcccaaccg cgaggggctg cgctactacc | 1560 |
| ggcggctgct ggagcggctg cgggagctgg gCgtgcagcc ggtggttacc ctgtaccatt | 1620 |
| gggacctgcc acagcgcctg caggacacct atggcggatg ggccaatcgc gccctggccg | 1680 |
| accatttcag ggattatgcc gagctctgct tccgccactt cggtggtcag gtcaagtact | 1740 |
| ggatcaccat tgacaacccc tacgtggtgg cctggcacgg gtatgccacc gggcgcctgg | 1800 |
| ccccgggcgt gaggggcagc tccaggctcg ggtacctggt tgcccacaac ctacttttgg | 1860 |
| ctcatgccaa agtctggcat ctctacaaca cctctttccg ccccacacag ggaggccggg | 1920 |
| tgtctatcgc cttaagctcc cattggatca atcctcgaag aatgactgac tataatatca | 1980 |
| gagaatgcca gaagtctctt gactttgtgc taggctggtt tcccaaaccc atatttattg | 2040 |
| atggcgacta cccagagagt atgaagaaca acctctcgtc tcttctgcct gattttactg | 2100 |
| aatctgagaa gaggctcatc agaggaactg ctgacttttt tgctctctcc ttcggaccaa | 2160 |
| ccttgagctt tcagctattg gaccctaaca tgaagttccg ccaattggag tctcccaacc | 2220 |
| tgaggcagct tctgtcttgg atagatctgg aatataacca ccctccaata tttattgtgg | 2280 |
| aaaatggctg gtttgtctcg ggaaccacca aaagggatga tcccaaatat atgtattatc | 2340 |
| tcaagaagtt cataatggaa accttaaaag caatcagact ggatggggtc gacgtcattg | 2400 |
| ggtacaccgc gtggtcgctc atggacggtt tcgagtggca taggggctac agcatccggc | 2460 |
| gaggactctt ctacgttgac tttctgagtc aggacaagga gctgttgcca aagtcttcgg | 2520 |
| ccttgttcta ccaaaagctg atagaggaca atggctttcc tcctttacct gaaaaccagc | 2580 |
| cccttgaagg gacatttccc tgtgactttg cttggggagt tcttgacaac tacgttcagc | 2640 |
| tgagtccttt gacaaaaccc agtgtcggcc tcttgcttcc tcactaa | 2687 |

In some embodiments, the gene construct includes the human desmin gene promoter operatively linked to a nucleic acid sequence encoding human s-KL (e.g., the nucleic acid sequence of SEQ ID NO: 3).

In some embodiments, the nucleic acid encoding the mammalian s-KL or a functional variant thereof is operatively linked to more than one, e.g., two, three, or four, different promoters all operatively linked to the nucleic acid encoding s-KL. In some embodiments, the additional promoter is also a muscle cell-specific promoter. In some embodiments, the additional promoter is a neuronal cell-specific promoter. In some embodiments, the additional promoter is a ubiquitous promoter. A “ubiquitous promoter” is a promoter that drives expression in virtually tissues. In some embodiments, the additional promoter is a constitutive promoter. In some embodiments, the additional promoter is an inducible promoter. In some embodiments, the additional promoter is a neuronal cell-specific promoter. In some embodiments, the gene construct contains three promoters; a muscle cell-specific first promoter, a neuronal cell-specific promoter, and an inducible promoter all operatively linked to the nucleic acid encoding s-KL.

In some embodiments, the additional promoter is a hybrid (synthetic) neuronal- and muscle-specific promoters may also be useful. Types of such promoters may include muscle-specific enhancers and/or transcription factor binding sites, including combinations with other promoter elements from viruses or human (e.g., CMV, CAG, or PGK). In some embodiments, the neuronal cell-specific promoter is a hybrid promoter, with one of the promoters described above fused to the CMV enhancer. Representative examples of hybrid promoters include a cytomegalovirus enhancer (CMV E-380 bp) 5′ to the PDGF-β promoter described in Liu et al., Gene Ther. 11 (1):52-60 (2004) and the CMV enhancer fused to the SYN promoter as described in Hioki et al., Gene Ther. 14 (11):872-882 (2007). Additional hybrid neuronal cell-specific promoters are described, for example, in U.S. Patent Application Publication 2009/0055941. Yet other examples of muscle-cell specific promoters include synthetic neuronal cell-specific promoters with activities higher than naturally occurring promoters

In other embodiments, the additional promoter is other than a tissue-specific promoter, representative types of which include constitutive promoters and inducible promoters. Constitutive promoters initiate RNA synthesis independently from regulatory influences. Inducible promoters allow regulation of gene expression and can be regulated by exogenously supplied compounds, environmental factors such as temperature, or the presence of a specific physiological state. Representative examples of specific promoters include CMV promoters (e.g., the cytomegalovirus intermediate-early (CMV IE) promoter), beta-actin promoters (e.g., chicken beta actin (CAG) promoters), CASI promoters (e.g., synthetic promoters described as a combination of the CMV enhancer, the chicken beta-actin promoter, and a splice donor and splice acceptor flanking the ubiquitin (UBC) enhancer, as described in U.S. Pat. No. 8,865,881), human phosphoglycerate kinase-1 (PGK) promoters, TBG promoters, retroviral Rous sarcoma virus LTR promoters, SV40 promoters, dihydrofolate reductase promoters, phosphoglycerol kinase (PGK) promoters, EF1a promoters, zinc-inducible sheep metallothionine (MT) promoters, dexamethasone (Dex)-inducible mouse mammary tumor virus (MMTV) promoters, T7 polymerase promoter systems, ecdysone insect promoters, tetracycline-repressible systems, tetracycline-inducible systems, RU486-inducible systems and rapamycin-inducible systems.

In some embodiments, the additional promoter is a zinc-driven inducible promoter. Representative examples of zinc-driven promoters include, the zinc-driven metallothionein promoter, the promoter/operator region of the zntA gene of Escherichia coli described in Brocklehurst et al., Mol. Microbiol. 31 (3):893-902 (1999), and the zinc-regulated promoters described in U.S. Pat. No. 8,354,272. In some embodiments, the additional promoter is a zinc-driven metallothionein promoter.

In some embodiments, the additional promoter that is operatively linked to the nucleic acid encoding mammalian s-KL or a functional variant thereof is a neuronal cell specific promoter. As used herein, the term “neuronal cell” embraces cells of the CNS, the PNS and the spinal cord, representative cell types of which include neurons (including motor neurons, sensory neurons and interneurons), glial cells, and astrocytes. Motor neurons signals from the brain and spinal cord to control everything from muscle contractions to glandular output. The terms “neuronal cell-specific” and “neuronal-specific” are used interchangeably herein and refer to the preferential, selective, or predominant expression of the mammalian s-KL (or functional variant thereof) in neuronal cells or neuronal tissue, as compared to other (i.e., non-neuronal) cells and tissues. In some embodiments, at least 50%, more particularly at least 60%, at least 70%, at least 75%, at least 80%, at least 85%, at least 90%, at least 95%, at least 98%, at least 99% or 100% of the expression occurs within neuronal cells or tissue. In some embodiments, “neuronal cell-specific” refers to no leakage of the expressed mammalian s-KL (or functional variant thereof) to other cells, organs, or tissue than neuronal, such as lung, liver, brain, kidney and/or spleen.

Representative examples of neuronal cell-specific promoters that may be suitable for use in the present disclosure include the synapsin 1 (SYN) promoter, the calcium/calmodulin-dependent protein kinase II promoter, the tubulin alpha I promoter, the neuron-specific enolase (NSE) promoter, the platelet-derived growth factor beta chain (PDGβ) promoter, the microtubule-associate protein 1B (MAP1B), the dopaminergic receptor 1 (Drd1a) promoter, the 67 kDa glutamic acid decarboxylase (GAD67) promoter, the homeobox Dlx5/6, glutamate receptor 1 (GluR1) promoter, the glial fibrillary acidic protein (GFAP) promoter, and the preprotachykinin 1 (Tac1) promoter.

Aside from the one or more promoters and to the extent they are not otherwise contained in an expression vector, the gene construct/expression cassette may further include one of more other non-coding, regulatory elements also known as expression control sequences. Representative examples of regulatory elements include include appropriate transcription initiation, termination, promoter and enhancer sequences; efficient RNA processing signals e.g., such as splicing and polyadenylation (poly A) tail sequences; poly A consensus sequences, tetracycline regulatable systems, posttranscriptional regulatory elements, sequences that stabilize cytoplasmic mRNA; sequences that enhance translation efficiency (e.g., Kozak consensus sequence); and sequences that enhance protein stability. A “posttranscriptional regulatory element,” as used herein, is a DNA sequence that, when transcribed, enhances the expression of the transgene(s) or fragments thereof that are delivered by viral vectors of the disclosure. Representative examples of posttranscriptional regulatory elements include the Hepatitis B Virus Postranscriptional Regulatory Element (HPRE) and the Woodchuck Hepatitis Postranscriptional Regulatory Element (WPRE). The WPRE is a tripartite cis-acting element that has been demonstrated to enhance transgene expression driven by certain, but not all promoters.

In some embodiments, the gene construct includes the cytomegalovirus intermediate-early (CMV IE) promoter operatively linked to the nucleic acid encoding a mammalian s-KL, the post-transcriptional regulatory element of the woodchuck hepatitis virus (WPRE) and a poly A.

In some embodiments, the gene construct includes a CAG promoter operatively linked to the nucleic acid encoding for s-KL (cDNA of mouse or human s-KL) and a poly A tail.

The gene construct may be in the form of DNA (e.g., for use with plasmids and various viral vectors (e.g., adenovirus and adeno-associated virus (AAV) vectors), or RNA (e.g., for linearized mRNA constructs or use with other viral vectors such as retroviral and lentiviral vectors). In some embodiments in which the gene construct is in the form of RNA for linearized mRNA constructs, the gene construct is incorporated into a plasmid, the plasmid is treated with enzymes (e.g., restriction enzymes) to yield a linear DNA, which is transcribed in a bioreactor as in vitro transcription to produce mRNA. The linearized mRNA is complexed with positively charged polymers, cationic lipids, or other complexes to form extruded nanoparticles, extruded micron-sized particles, or micellar emulsions.

In some embodiments in which the gene construct is in the form of RNA for retroviral and lentiviral delivery, the gene construct contains the Group Antigen (gag), reverse transcriptase (pol), and envelope (env) genes, as well as 5′ and 3′ long terminal repeats (LTR). In some embodiments, the 5′ LTR is a chimeric 5′ LTR that contains a heterologous promoter that does not rely on transactivation by the lentiviral tat protein. Lentiviral LTRs may be divided into three elements, designated U3, R, and U5. The U3 element is unique to the 3′ end of the lentiviral RNA genome. R is repeated at both ends of the lentiviral RNA genome, and U5 is unique to the 5′ end of the lentiviral RNA genome. The size of the three elements can vary significantly among different viruses. In some embodiments, portions of either LTR are modified, replaced, or deleted, for example, a portion of the 3′ U3 is deleted. In one embodiment, the 3′ U5 element is replaced with a poly A. In one embodiment, the 5′ U3 is replaced with a truncated CMV immediate early (IE) enhancer/TATA promoter. Additional lentiviral non-coding regulator elements are known in the art, see, e.g., WO 2021/181108A1, and WO 2021/160993A1 and U.S. Pat. Nos. 6,669,936, 6,924,123 and 10,544,429.

In some embodiments, this gene construct (or nucleic acid construct) further comprises one or more of:
(a) a poly A signal, e.g., an SV40 poly A tail;
(b) a protein translation initiation site consensus nucleic acid;
(c) a post-transcriptional regulatory element nucleic acid;
(d) 5′ and 3′ inverted terminal repeats; and
(e) an intron.

As known in the art, the term “intron” encompasses any portion of a whole intron that is large enough to be recognized and spliced by the nuclear splicing apparatus. Typically, short, functional, intron sequences are preferred in order to keep the size of the expression cassette as small as possible which facilitates the construction and manipulation of the expression cassette. In some embodiments, the intron is obtained from a gene that encodes the protein that is encoded by the coding sequence within the expression cassette. The intron can be located 5′ to the coding sequence, 3′ to the coding sequence, or within the coding sequence. An advantage of locating the intron 5′ to the coding sequence is to minimize the chance of the intron interfering with the function of a polyadenylation signal. Representative examples of suitable introns include Minute Virus of Mice (MVM) intron, beta-globin intron (betaIVS-1), factor IX (FIX) intron A, Simian virus 40 (SV40) small-t intron, and beta-actin intron.

All these elements (a) to (e) are sequences that allow the proper expression of the gene operatively linked to a promoter. The skilled person in the art will understand which sequences are referred as well as their function.

In some embodiments, the gene construct may include a muscle-specific regulatory element, e.g., other than a muscle cell-specific promoter, that may enhance muscle-specific expression of the mammalian s-KL coding sequence. Representative examples of such sequences include the CSk-SH1, CSk-SH2, CSk-SH3, CSk-SH4, CSk-SH51, and CSk-SH6 regulatory elements. See, e.g., U.S. Patent Application Publication 2020/00407746. In some embodiments, such regulatory element may be situated upstream of the promoter.

The nucleic acid sequence of a gene construct comprising a nucleic acid encoding SEQ ID NO: 6, a WPRE and a poly A is set forth below as (SEQ ID NO: 7):

| | |
|---|---|
| cagcagctgc gcgctcgctc gctcactgag gccgcccggg caaagcccgg gcgtcgggcg | 60 |
| acctttggtc gcccggcctc agtgagcgag cgagcgcgca gagagggagt ggccaactcc | 120 |
| atcactaggg gttccttgta gttaatgatt aacccgccat gctacttatc tacgtagcca | 180 |
| tgctctagac atggctcgac agatctgcgc gcgatcgata tcagcgcttt aaatttgcgc | 240 |
| atgctagcgc ggccgcaccc atgcctcctc aggtaccccc tgccccccac agctcctctc | 300 |
| ctgtgccttg tttcccagcc atgcgttctc ctctataaat acccgctctg gtatttgggg | 360 |
| ttggcagctg ttgctgccag ggagatggtt gggttgacat gcggctcctg acaaaacaca | 420 |
| aacccctggt gtgtgtgggc gtgggtggtg tgagtagggg gatgaatcag ggagggggcg | 480 |
| ggggacccag ggggcaggag ccacacaaag tctgtgcggg ggtgggagcg cacatagcaa | 540 |
| ttggaaactg aaagcttatc agaccctttc tggaaatcag cccactgttt ataaacttga | 600 |
| ggccccaccc tcgagataac cagggctgaa agaggcccgc ctgggggctg cagacatgct | 660 |
| tgctgcctgc cctggcgaag gattggcagg cttgcccgtc acaggacccc cgctggctga | 720 |
| ctcaggggcg caggcctctt gcgggggagc tggcctcccc gcccccacgg ccacgggccg | 780 |
| ccctttcctg gcaggacagc gggatcttgc agctgtcagg ggaggggagg cgggggctga | 840 |
| tgtcaggagg gatacaaata gtgccgacgg ctgggggccc tgtctcccct cgccgcatcc | 900 |
| actctccggc cggccgcctg cccgccgcct cctccgtgcg cccgccagcc tcgcccgcgc | 960 |
| cgtcaccggt tcgaacaggt aagcgcccct aaaatccctt tcgcacaatg tgtcctgagg | 1020 |
| ggagaggcag cgacctgtag atgggacggg ggcactaacc ctcagggttt ggggttctga | 1080 |
| atgtgagtat ccccatctaa gcccagtatt tcgccaatct cagaaagctc ctggctccct | 1140 |
| ggaggatgga gagagaaaaa caaacagctc ctggagcagg gagagtgttg gcctcttgct | 1200 |
| ctccggctcc ctctgttgcc ctctggtttc tccccaggtt cgaaggatcc actagtccag | 1260 |
| tgtggtggaa ttcgccgcca ccatgctagc ccgcgcccct cctcgccgcc cgccgcggct | 1320 |
| ggtgctgctc cgtttgctgt tgctgcatct gctgctgctc gccctgcgcg cccgctgcct | 1380 |
| gagcgctgag ccgggtcagg gcgcgcagac ctgggctcgc ttcgcgcgcg ctcctgcccc | 1440 |
| agaggccgct ggcctcctcc acgacacctt ccccgacggt ttcctctggg cggtaggcag | 1500 |
| cgccgcctat cagaccgagg gcggctggcg acagcacggc aaaggcgcgt ccatctggga | 1560 |
| cactttcacc catcactctg gggcggcccc gtccgactcc ccgatcgtcg tggcgccgtc | 1620 |
| gggtgccccg tcgcctcccc tgtcctccac tggagatgtg gccagcgata gttacaacaa | 1680 |
| cgtctaccgc gacacagagg ggctgcgcga actgggggtc acccactacc gcttctccat | 1740 |
| atcgtgggcg cgggtgctcc ccaatggcac cgcgggcact cccaaccgcg aggggctgcg | 1800 |
| ctactaccgg cggctgctgg agcggctgcg ggagctgggc gtgcagccgg tggttaccct | 1860 |
| gtaccattgg gacctgccac agcgcctgca ggacacctat ggcggatggg ccaatcgcgc | 1920 |
| cctggccgac catttcaggg attatgccga gctctgcttc cgccacttcg gtggtcaggt | 1980 |
| caagtactgg atcaccattg acaaccccta cgtggtggcc tggcacgggt atgccaccgg | 2040 |
| gcgcctggcc ccgggcgtga ggggcagctc caggctcggg tacctggttg cccacaacct | 2100 |
| acttttggct catgccaaag tctggcatct ctacaacacc tctttccgcc ccacacaggg | 2160 |
| aggccgggtg tctatcgcct taagctccca ttggatcaat cctcgaagaa tgactgacta | 2220 |
| taatatcaga gaatgccaga agtctcttga ctttgtgcta ggctggtttg ccaaacccat | 2280 |
| atttattgat ggcgactacc cagagagtat gaagaacaac ctctcgtctc ttctgcctga | 2340 |
| ttttactgaa tctgagaaga ggctcatcag aggaactgct gacttttttg ctctctcctt | 2400 |
| cggaccaacc ttgagctttc agctattgga ccctaacatg aagttccgcc aattggagtc | 2460 |
| tcccaacctg aggcagcttc tgtcttggat agatctggaa tataaccacc ctccaatatt | 2520 |
| tattgtggaa aatggctggt ttgtctcggg aaccaccaaa agggatgatg ccaaatatat | 2580 |
| gtattatctc aagaagttca taatggaaac cttaaaagca atcagactgg atggggtcga | 2640 |
| cgtcattggg tacaccgcgt ggtcgctcat ggacggtttc gagtggcata ggggctacag | 2700 |
| catccggcga ggactcttct acgttgactt tctgagtcag gacaaggagc tcttgccaaa | 2760 |
| gtcttcggcc ttgttctacc aaaagctgat agaggacaat ggctttcctc ctttacctga | 2820 |
| aaaccagccc cttgaaggga catttccctg tgactttgct tggggagttg ttgacaacta | 2880 |
| cgttcagctg agtcctttga caaaacccag tgtcggcctc ttgcttcctc actaagggcc | 2940 |
| gcttaatacg actcactata gggaccggtg gcgcgcctga tcattcgaag gccggccgaa | 3000 |
| ttcacgcgtg cggccgcaag aattcgatat caagcttatc gataatcaac ctctggatta | 3060 |
| caaaatttgt gaaagattga ctggtattct taactatgtt gctcctttta cgctatgtgg | 3120 |
| atacgctgct ttaatgcctt tgtatcatgc tattgcttcc cgtatggctt tcattttctc | 3180 |
| ctccttgtat aaatcctggt tgctgtctct ttatgaggag ttgtggcccg ttgtcaggca | 3240 |
| acgtggcgtg gtgtgcactg tctttgctga cgcaaccccc actggttggg gcattgccac | 3300 |
| cacctgtcag ctcctttccg ggactttcgc tttccccctc cctattgcca cggcggaact | 3360 |
| catcgccgcc tgccttgccc gctgctggac aggggctcgg ctgttgggca ctgacaattc | 3420 |
| cgtggtgttg tcggggaaat catcgtcctt tccttggctg ctcgcctgtg ttgccacctg | 3480 |
| gattctgcgc gggacgtcct tctgctacgt cccttcggcc ctcaatccag cggaccttcc | 3540 |
| ttcccgcggc ctgctgccgg ctctgcggcc tcttccgcgt cttcgccttc gccctcagac | 3600 |
| gagtcggatc tccctttggg ccgcctcccc gcatcgatac cgtctacagg ccttcgcgat | 3660 |
| taattaagtt taaaccatat gatcgataca tgtgtttact ccggaatatt aataggccta | 3720 |
| ggatgcatat ggcggccgct tccctttagt gagggttaat gcttcgagca gacatgataa | 3780 |
| gatacattga tgagtttgga caaaccacaa ctagaatgca gtgaaaaaaa tgctttattt | 3840 |
| gtgaaattty tgatgctatt gctttatttg taaccattat aagctgcaat aaacaagtta | 3900 |
| acaacaacaa ttgcattcat tttatgtttc aggttcaggg ggagatgtgg gaggtttttt | 3960 |
| aaagcaagta aaacctctac aaatgtggta aaatccgata agggactaga gcatggctac | 4020 |
| gtagataagt agcatggcgg gttaatcatt aactacaagg aacccctagt gatggagttg | 4080 |
| gccactccct ctctgcgcgc tcgctcgctc actgaggccg ggcgaccaaa ggtcgcccga | 4140 |
| cgcccgggct ttgcccgggc ggcctcagtg agcgagcgag cgcgccagct ggcgtaatag | 4200 |
| cgaagaggcc cgcaccgatc gcccttccca acagttgcgc agcctgaatg gcgaatggaa | 4260 |
| ttccagacga ttgagcgtca aaatgtaggt atttccatga gcgtttttcc gttgcaatgg | 4320 |
| ctggcggtaa tattgttctg gatattacca gcaaggccga tagtttgagt tcttctactc | 4380 |
| aggcaagtga tcttattact aatcaaagaa gtattgcgac aacggttaat ttgcgtgatg | 4440 |
| gacagactct tttactcggt ggcctcactg attataaaaa cacttctcag gattctggcg | 4500 |
| taccgttcct gtctaaaatc cctttaatcg gcctcctgtt tagctcccgc tctgattcta | 4560 |
| acgaggaaag cacgttatac gtgctcgtca aagcaaccat agtacgcgcc ctgtagcggc | 4620 |
| gcattaagcg cggcgggtgt ggtggttacg cgcagcgtga ccgctacact tcccagcgcc | 4680 |
| ctagcgcccg ctcctttcgc tttcttccct tcctttctcg ccacgttcgc cggctttccc | 4740 |
| cgtcaagctc taaatcgggg gctcccttta gggttccgat ttagtgcttt acggcacctc | 4800 |
| gaccccaaaa aacttgatta gggtgatggt tcacgtagtg ggccatcgcc ctgatagacg | 4860 |
| gtttttcgcc ctttgacgtt ggagtccacg ttctttaata gtggactctt gttccaaact | 4920 |
| ggaacaacac tcaaccctat ctcggtctat tcttttgatt tataagggat tttgccgatt | 4980 |
| tcggcctatt gcttaaaaaa tgagctgatt taacaaaaat ttaacgcgaa ttttaacaaa | 5040 |
| atattaacgt ctacaattta aatatttgct tatacaatct tcctgttttt ggggcttttc | 5100 |
| tgattatcaa ccggggtaca tatgattgac atgctagttt tacgattacc gttcatcgat | 5160 |
| tctcttgttt gctccagact ctcaggcaat gacctgatag cctttgtaga gacctctcaa | 5220 |
| aaatagctac cctctccggc atgaatttat cagctagaac gcttgaatat catattgatg | 5280 |
| gtgatttgac tgtctccggc ctttctcacc cgtttgaatc tttacctaca cattactcag | 5340 |
| gcattgcatt taaaatatat gagggttcta aaaattttta tccttgcgtt gaaataaagg | 5400 |
| cttctcccgc aaaagtatta cagggtcata atgtttttgg tacaaccgat ttagctttat | 5460 |
| gctctgaggc tttattgctt aattttgcta attctttgcc ttgcctgtat gatttattgg | 5520 |
| atgttggaat cgcctgatgc ggtattttct ccttacgcat ctgtgcggta tttcacaccg | 5580 |
| catatggtgc actctcagta caatctgctc tgatgccgca tagttaagcc agccccgaca | 5640 |
| cccgccaaca cccgctgacg cgccctgacg ggcttgtctg ctcccggcat ccgcttacag | 5700 |
| acaagctgtg accgtctccg ggagctgcat gtgtcagagg ttttcaccgt catcaccgaa | 5760 |
| acgcgcgaga cgaaagggcc tcgtgatacg cctattttta taggttaatg tcatgataat | 5820 |
| aatggtttct tagacgtcag gtggcacttt tcggggaaat gtgcgcggaa cccctatttg | 5880 |
| tttatttttc taaatacatt caaatatgta tccgctcatg agacaataac cctgataaat | 5940 |
| gcttcaataa tattgaaaaa ggaagagtat gagtattcaa catttccgtg tcgcccttat | 6000 |
| tccctttttt gcggcatttt gccttcctgt ttttgctcac ccagaaacgc tggtgaaagt | 6060 |
| aaaagatgct gaagatcagt tcggtgcacg agtgggttac atcgaactgg atctcaacag | 6120 |
| ccgtaagatc cttgagagtt ttcgccccga agaacgtttt ccaatgatga gcacttttaa | 6180 |
| agttctgcta tgtggcgcgg tattatcccg tattgacgcc gggcaagagc aactcggtcg | 6240 |
| ccgcatacac tattctcaga atgacttggt tgagtactca ccagtcacag aaaagcatct | 6300 |
| tacggatggc atgacagtaa gagaattatg cagtgctgcc ataaccatga gtgataacac | 6360 |
| tgcggccaac ttacttctga caacgatcgg aggaccgaag gagctaaccg cttttttgca | 6420 |
| caacatgggg gatcatgtaa ctcgccttga tcgttgggaa ccggagctga atgaagccat | 6480 |
| accaaacgac gagcgtgaca ccacgatgcc tgtagcaatg gcaacaacgt tgcgcaaact | 6540 |
| attaactggc gaactactta ctctagcttc ccggcaacaa ttaatagact ggatggaggc | 6600 |
| ggataaagtt gcaggaccac ttctgcgctc ggcccttccg gctggctggt ttattgctga | 6660 |
| taaatctgga gccggtgagc gtgggtctcg cggtatcatt gcagcactgg ggccagatgg | 6720 |
| taagccctcc cgtatcgtag ttatctacac gacggggagt caggcaacta tggatgaacg | 6780 |
| aaatagacag atcgctgaga taggtgcctc actgattaag cattggtaac tgtcagacca | 6840 |
| agtttactca tatatacttt agattgattt aaaacttcat ttttaattta aaaggatcta | 6900 |
| ggtgaagatc ctttttgata atctcatgac caaaatccct taacgtgagt tttcgttcca | 6960 |
| ctgagcgtca gaccccgtag aaaagatcaa aggatcttct tgagatcctt tttttctgcg | 7020 |
| cgtaatctgc tgcttgcaaa caaaaaaacc accgctacca gcggtggttt gtttgccgga | 7080 |
| tcaagagcta ccaactcttt ttccgaaggt aactggcttc agcagagcgc agataccaaa | 7140 |
| tactgtcctt ctagtgtagc cgtagttagg ccaccacttc aagaactctg tagcaccgcc | 7200 |
| tacatacctc gctctgctaa tcctgttacc agtggctgct gccagtggcg ataagtcgtg | 7260 |
| tcttaccggg ttggactcaa gacgatagtt accggataag gcgcagcggt cgggctgaac | 7320 |
| ggggggttcg tgcacacagc ccagcttgga gcgaacgacc tacaccgaac tgagatacct | 7380 |
| acagcgtgag ctatgagaaa gcgccacgct tcccgaaggg agaaaggcgg acaggtatcc | 7440 |
| ggtaagcggc agggtcggaa caggagagcg cacgagggag cttccagggg gaaacgcctg | 7500 |
| gtatctttat agtcctgtcg ggtttcgcca cctctgactt gagcgtcgat ttttgtgatg | 7560 |
| ctcgtcaggg gggcggagcc tatggaaaaa ccccagcaac gcggcctttt tacggttcct | 7620 |
| ggccttttgc tggccttttg ctcacatgtt ctttcctgcg ttatcccctg attctgtgga | 7680 |
| taaccgtatt accgcctttg agtgagctga taccgctcgc cgcagccgaa cgaccgagcg | 7740 |
| cagcgagtca gtgagcgagg aagcggaaga gcgcccaata cccaaaccgc ctctccccgc | 7800 |
| gcgttggccg attcattaat g | 7821 |

Methods of cloning nucleic acid and gene constructs into vectors are known in the art. In some embodiments one or more sequences, or the entire gene construct may be codon-optimized, e.g., depleted of CpG dinucleotides. In one embodiment, the promoter of the gene construct is completely depleted of CpG dinucleotides. CpG dinucleotide depletion may be achieved by site directed mutagenesis, in vitro gene construct synthesis, or by any appropriate molecular biology technique.

### Expression Vectors

An expression vector, also known as an expression construct, is typically a virus or a plasmid (e.g., which may contain a viral genome or portions thereof) designed for protein expression in cells. An expression vector has features that any vector may have, such as an origin of replication, a selectable marker, and a suitable site for the insertion of the gene construct, such as a multiple cloning site (MCS).

In one aspect, the disclosure provides an expression vector, comprising: (a) a nucleic acid construct comprising a muscle cell-specific promoter operatively linked to a nucleic acid encoding a mammalian s-KL or function variant thereof, wherein the vector may or may not have muscle cell tropism; or (b) a nucleic acid construct comprising a first promoter functionally operable in a muscle cell operatively linked to a nucleic acid encoding a mammalian s-KL or function variant thereof, wherein the expression vector has a muscle cell tropism.

### Viral Expression Vectors

In some embodiments, the expression vector is a viral vector, for example, a retroviral vector, a lentivirus vector, an adenoviral vector, a herpesvirus vector, an adenovirus, or an adeno-associated virus (AAV) vector.

The term “adeno-associated virus” as used herein refers to a viral vector that infects both dividing and quiescent primate (and human) cells. Lacking any pathogenic effects, and usually integrate in the same place of the genome (the AAVS1 site, in chromosome 19), this viral vector can safely be used to transduce foreign DNA into human cells in gene therapy applications.

In some embodiments, the expression vector is an adeno-associated virus (AAV). AAV vectors may be derived from any suitable host species, including human (h), baboons, chimpanzees, and rhesus (rh), pigtailed (pi), and cynomolgus macaques. The genomic organization of all known AAV serotypes is similar. The genome of AAV is a linear, single-stranded DNA molecule that is less than about 5,000 nucleotides (nt) in length. Inverted terminal repeats (ITRs) flank the unique coding nucleotide sequences for the non-structural replication (Rep) proteins and the structural (VP) proteins. The VP proteins (VP1, -2 and -3) form the capsid and contribute to the tropism of the virus. The terminal 145 nt ITRs are self-complementary and are organized so that an energetically stable intramolecular duplex forming a T-shaped hairpin may be formed. These hairpin structures function as an origin for viral DNA replication, serving as primers for the cellular DNA polymerase complex. Following wild-type (wt) AAV infection in mammalian cells the Rep genes are expressed and function in the replication of the viral genome.

Representative examples of AAV expression vectors may be derived from serotypes of AAVs including AAV1, AAV3, AAV2, AAV4, AAV5, AAV6, AAV7, AAV8, AAV9, AAVrh10, AAVrh.43, AAVrh74, AAVpi.2, AAVrh.8, AAVhu.11, AAVhu.32, and AAVhu.37, PHPeB, 9P31, and AAVmyo. AAV serotype 9 (AAV9) is known to achieve efficient transduction in heart and skeletal muscle. Accordingly, in some embodiments, the expression vector is an AAV9 vector, e.g., a self-complementary AAV9 vector (scAAV9).

In some embodiments, the expression vector is an AAV of a serotype with muscle cell tropism. The term “muscle cell tropism” as used herein refers to a vector that preferentially affects muscle cells, for example, a viral vector that preferentially infects muscle cells over other cell types in the body. Representative examples of such AAVs of a serotype with muscle cell tropism include AAV1, AAV5, AAV6, AAV8, AAV9 (BBB), AAVrh.74, and AAVmyo. Additional AAVs with muscle cell tropisms are known in the art. See, U.S. Patent Application Publication 2021/0363193; Tabebordbar et al., Cell 184 (19):4919-4938 (2021); and Weinmann et al., Nat. Commun. 11 (1):5432 (2020). In some embodiments, the expression vector is an adeno-associated virus of a serotype with muscle cell tropism selected from AAV8 and AAVmyo. In these embodiments, the promoter need not be a muscle cell-specific promoter.

In some embodiments, the expression vector is an adeno-associated virus containing an AAV capsid polypeptide that comprises or consists in the amino acid sequence of any one of SEQ ID NOs: 18-29 or is encoded by the nucleic acid having the sequence of any one of SEQ ID NOs: 30-41. See, Grimm, et al., U.S. Patent Application Publication 2021/0363193.

In some embodiments, the AAV capsid polypeptide comprises or consists in the amino acid sequence of SEQ ID NO: 17 which is encoded by the nucleic acid sequence of SEQ ID NO: 30, and which corresponds to AAV9P1 disclosed in U.S. Patent Application Publication 2021/0363193. In some embodiments, the AAV capsid polypeptide comprises or consists in the amino acid sequence of SEQ ID NO: 26 which is encoded by the nucleic acid sequence of SEQ ID NO: 38, and which corresponds to AAV9S10P1 disclosed in U.S. Patent Application Publication 2021/0363193. In some embodiments, the AAV capsid polypeptide comprises or consists in the amino acid sequence of SEQ ID NO: 27 which is encoded by the nucleic acid sequence of SEQ ID NO: 39, and which corresponds to AAVS1P1 disclosed in U.S. Patent Application Publication 2021/0363193.

In some embodiments, the expression vector is an adeno-associated virus of a AAV8 serotype, which comprises or consists in the nucleic acid sequence of SEQ ID NO: 7, which sequence is a gene construct comprising the human desmin promoter, the nucleic acid coding for mouse s-KL, the WPRE sequence and the SV40 poly A tail.

In some embodiments, the expression vector is an adeno-associated virus of a AAV8 serotype that comprises or consists in the nucleic acid sequence of SEQ ID NO: 7, but in which the gene encodes human s-KL, for example the nucleic acid sequence of SEQ ID NO: 3.

The positions of the different sequences in SEQ ID NO: 7 are illustrated in Table A.

TABLE A
Sequence legend for SEQ ID NO: 7.

| Feature | Location (bp or nt) | Size |
|---|---|---|
| Human Desmin promoter | 249 . . . 1250 | 1002 bp |
| Mouse Secreted Klotho | 1283 . . . 2935 | 1686 bp |
| WPRE | 3038 . . . 3632 | 595 bp |
| SV40 polyA | 3778 . . . 3899 | 122 bp |

In some embodiments, the expression vector is an AAV of a serotype with a neuronal tropism. The term “neuronal cell tropism” as used herein refers to a vector that preferentially affects neuronal cells, for example, a viral vector that preferentially infects motor neurons over other cell types in the body. Representative examples of such AAVs of a serotype with neuronal cell tropism include AAV1, AAV6, and AAV7. In some embodiments, the expression vector is an adeno-associated virus of a serotype with neuronal cell tropism specific for astrocytes, e.g., AAV5. In some embodiments, expression vector is a vector with a neuronal cell tropism as described, for example, in U.S. Patent Application Publications 2019/0030138 and 2020/0339960. In these embodiments, the promoter need not be a neuronal-specific promoter.

In some embodiments, the expression vector is an AAV delivered intraparenchymal injection targeting the spinal cord and is of a serotype with a neuronal cell tropism. In some of these embodiments, the AAV serotype is AAV1, AAV5, AAV9. In some embodiments, AAV expression vectors are delivered intravenously and have neuronal cell tropism. AAV vector serotypes with neuronal cell tropisms well adapted for intravenous injection include AAV9, AAVhr.10, AAVrh.8, and AAVrh43.

In some embodiments, the expression vector is configured to undergo axonal retrograde transport. In some of these embodiments, the expression vector is an AAV serotype of AAV1, AAV5, AAV8, AAV9, or AAVrh.10. In one embodiment, an AAV1 serotype expression vector is injected into muscle or sciatic nerve and preferentially targets motor neurons.

In some embodiments, the viral vector is a recombinant AAV (rAAV) comprising at least one capsid protein from an AAV serotype. AAV capsid proteins may provide tissue-specific targeting, such that the AAV will infect and deliver the gene construct to the desired tissue or organ. In some embodiments, the AAV is pseudotyped AAV (pAAV) comprising virus or viral vector that has viral envelope proteins from more than one virus. pAAV may have altered host or tissue tropisms or increased or decreased particle stability. In some embodiments, pAAV comprises nucleic acids from two or more different AAVs, for example, the nucleic acid from one AAV source encodes a capsid protein and the nucleic acid of at least one other AAV source encodes other viral proteins and/or the viral genome. In some embodiments, a pAAV refers to an AAV comprising an inverted terminal repeats (ITRs) of one AAV serotype and a capsid protein of a different AAV serotype. For example, a pAAV vector containing the ITRs of serotype X encapsulated with the proteins of Y will be designated as AAVX/Y (e.g., AAV2/1 has the ITRs of AAV2 and the capsid of AAV1). In some embodiments, pAAVs may be useful for combining the tissue-specific targeting capabilities of a capsid protein from one AAV serotype with the viral DNA (e.g., viral ITRs) from another AAV serotype, thereby allowing targeted delivery of a transgene to a target tissue.

AAV vector production is most often achieved with a double plasmid transfection followed by a helper adenovirus infection in vitro. The two plasmids of the double plasmid transfection are a vector plasmid and a packaging plasmid. The vector plasmid contains the gene construct described herein flanked by the AAV inverted terminal repeats (ITR), while the packaging plasmid provides AAV replicase (Rep) and capsid (Cap) genes for vector DNA replication and packaging. Once a suitable cell line (e.g., HeLa) is transfected with the two plasmids, it is then infected with the helper adenovirus, and the AAV genes are expressed, the genes from both plasmids are expressed and packaged into AAV particles. The proteins encoded on the packaging plasmid make up the AAV particle, while the vector plasmid (containing the gene construct) is packaged into the AAV particle. AAV particles are either purified away from helper adenovirus particles, or the adenovirus particles are selectively heat inactivated. This procedure produces replication deficient AAV containing the gene construct which may be further formulated and delivered to a subject.

Alternatively, in place of helper adenovirus infection in vitro, a third adenovirus-derived plasmid containing the necessary replication genes from adenovirus (e.g., E1A, E1B, E2A, E4, and VA RNA) may also be transfected along with the vector plasmid and the packaging plasmid. In some embodiments, the packaging and adenovirus-derived plasmids may be stably integrated into the cell line.

Additional methods for producing and delivering AAVs, rAAVs and pAAVs are known in the art. See, e.g., U.S. Patent Publications 2015/0065560, 2013/0090374, and 2012/0309050, and U.S. Pat. Nos. 11,041,171, 11,020,443, and 7,858,367.

In some embodiments, the expression vector is a lentivirus vector or a recombinant lentivirus vector. In some embodiments, the expression vector is a non-integrative and non-replicative recombinant lentivirus vector. The construction of lentiviral vectors has been described, for example, in U.S. Pat. Nos. 5,665,577, 5,981,276, 6,013,516, 7,090,837, 8,119,119 and 10,954,530. Lentivirus vectors include a defective lentiviral genome, i.e., in which at least one of the lentivirus genes gag, pol, and env, has been inactivated or deleted. Additional lentivirus genes and sequences include the Rev response elements (RRE) and the DNA flap at the center of the viral cDNA in between the central polypurine tract (cPPT) and the central termination sequence (CTS), often abbreviated as cPPT CTS.

In some embodiments, the expression vector is a recombinant lentivirus comprising a recombinant genome comprising, between the LTR 5′ and 3′ lentiviral sequences, a lentiviral encapsulation psi sequence, an RNA nuclear export element, a transgene, and a promoter and/or a sequence favoring the nuclear import of RNA, as well as a mutated integrase preventing the integration of its genome into the genome of an isolated cell. A lentivirus vector can include, for example, the sequence 5′LTR-psi-RRE-cPPT CTS-transgene-LTR3′.

In some embodiments, viral vectors encoding the nucleic acid sequences of the present disclosure are administered along with empty vectors, comprising capsid proteins without any nucleic acid sequences of the present disclosure. In some embodiments, the empty vectors are administered in a ratio between 5:1 to 3:1 empty vector to viral vector to immunomodulate a subject's immune response against the viral rector.

In some embodiments, the viral vector is immunomodulated. Immunomodulation may be achieved by adding polyethylene glycol (PEG) to the viral vectors, typically by pegylation of lysine residues exposed on the surface of viral vectors. See, for example, U.S. Patent Application Publication 2021/0139860 and U.S. Pat. Nos. 6,399,385 and 10,022,457. Pegylation results in longer circulation time for the viral vector particles as well as reduced immune responses (i.e., lowered immunogenicity).

Additional viral vectors may be used in combination with the viral vectors disclosed herein. For example, the viral vectors described in U.S. Patent Application Publication 2019/0030138 may be combined with a viral vector described herein.

### Non-Viral Expression Vectors

In other embodiments, the expression vector is a non-viral vector, representative examples of which include plasmids, minicircles, and transposon-based vectors, such as Sleeping Beauty (SB)-based vectors and piggyBac(PB)-based vectors. In yet other embodiments, the vector may include both viral and non-viral elements.

In some embodiments the gene construct is incorporated into a plasmid expression vector. The plasmid may contain sequences encoding the gene construct (promoter and mammalian s-KL sequence), an initiation sequence, a poly-A tail sequence, optional regulatory elements, and other optional sequences (e.g., a multi-cloning site). In some embodiments, mRNA is produced from a circular plasmid. The plasmid may be linearized with restriction enzymes, in vitro transcribed to produce mRNA, modified with a 5′ cap and 3′ poly-A tail.

In some embodiments, a carrier encapsulates the gene construct or plasmid. The carrier may be lipid-based, e.g., lipid nanoparticles (LNPs), liposomes, lipid vesicles, or lipoplexes. In some embodiments, the carrier is an LNP. In certain embodiments, an LNP includes two or more concentric bilayers separated by aqueous compartments. Lipid bilayers may be functionalized and/or crosslinked to one another. Lipid bilayers may include one or more ligands, proteins, or channels.

Lipid carrier, e.g., LNPs may include one or more cationic/ionizable lipids, one or more polymer conjugated lipids, one or more structural lipids, and/or one or more phospholipids. A “cationic lipid” refers to positively charged lipid or a lipid capable of holding a positive charge. Cationic lipids include one or more amine group(s) which bear the positive charge, depending on pH. A “polymer conjugated lipid” refers to a lipid with a conjugated polymer portion. Polymer conjugated lipids include a pegylated lipids, which are lipids conjugated to polyethylene glycol. A “structure lipid” refers to a non-cationic lipid that does not have a net charge at physiological pH. Exemplary structural lipids include cholesterol, fecosterol, sitosterol, ergosterol, campesterol, and the like. A “phospholipid” refers to lipids that have a triester of glycerol with two fatty acids and one phosphate ion. Phospholipids in LNPs assemble the lipids into one or more lipid bilayers. LNPs, their method of preparation, formulation, and delivery are disclosed in, e.g., U.S. Pat. Nos. 9,364,435, 9,518,272, 10,022,435, and 11,191,849 and U.S. Patent Application Publication 2004/0142025, 2007/0042031, and 2020/0237679.

Lipoplexes, liposomes, and lipid nanoparticles may include a combination of lipid molecules, e.g., a cationic lipid, a neutral lipid, an anionic lipid, polypeptide-lipid conjugates, and other stabilization components. Representative stabilization components include antioxidants, surfactants, and salts. Compositions and preparation methods of lipoplexes, liposomes, and lipid nanoparticles are known in the art. See, e.g., U.S. Pat. Nos. 8,058,069, 8,969,353, 9,682,139, 10,238,754, U.S. Patent Application Publications 2005/0064026 and 2018/0291086, and Lasic, Trends Biotechnol. 16 (7):307-21 (1998), Lasic et al., FEBS Lett. 312 (2-3):255-8 (1992), and Drummond et al., Pharmacol. Rev. 51 (4):691-743 (1999).

### Pharmaceutical Compositions

The pharmaceutical compositions containing the expression vector of the disclosure may, in some embodiments, be formulated with a pharmaceutically acceptable carrier and optionally, a pharmaceutically acceptable excipient (collectively a pharmaceutically acceptable “vehicle”), for administration to a subject, via any suitable and medically acceptable mode of administration. In some embodiments, administration is a via parenteral (e.g., intravenous) delivery.

The expressions “pharmaceutically acceptable carrier” and “pharmaceutically acceptable excipient” refer to ingredients that are physiologically inert (e.g., non-immunogenic) and non-toxic, and which are compatible with all other ingredients of the pharmaceutical composition, and otherwise suitable for contact with the tissue or organ of subjects (e.g., mammals including both humans and non-human animals) commensurate with a reasonable benefit/risk ratio.

In some embodiments, the expression vector is formulated for purposes of systemic e.g., parenteral administration. In some embodiments, the carrier is aqueous, representative examples of which include water, saline solutions (e.g., physiological saline, bacteriostatic water, and phosphate buffered saline (PBS)), and aqueous dextrose and glycerol solutions. The aqueous carrier may also include a polyol such as glycerol, propylene glycol, and liquid polyethylene glycol, and the like. Aqueous-based compositions may comprise additional excipients, such as solubilizers, isotonic agents, suspending agents, emulsifying agents, stabilizers and preservatives. Such liquid-based formulations may be in the form of solutions, suspensions or dispersions. In some embodiments, the carrier is non-aqueous.

The pharmaceutical composition may further contain an excipient. As in the case of the carrier, the choice of excipient will be determined in part by the particular vector, as well as by the particular method used to administer the composition. Accordingly, persons skilled int the art will readily appreciate that there is a wide variety of suitable formulations of the pharmaceutical composition of the present disclosure. Representative examples of excipients include coating agents, surfactants and emulsifying agents, antimicrobial agents and other preservatives, solubilizers, isotonic agents, absorption blockers and suspending agents. Lecithin is an exemplary coating agent that establishes a proper fluidity and maintains the required particle size in the case of dispersions. Surfactants may also aid in particle size maintenance. Exemplary antimicrobial agents include antibacterial and antifungal agents, e.g., parabens, chlorobutanol, phenol, ascorbic acid, thimerosal, and the like. Exemplary isotonic agents include sugars and polyalcohols (e.g., manitol, sorbitol, and sodium chloride). Exemplary absorption blockers include aluminium monostearate and gelatin which prolong absorption of a gene construct in the composition.

Additional pharmaceutically acceptable excipients used in the manufacture of pharmaceutical compositions, their formulation, and delivery of expression vectors such as viral vectors are known in the art. See, for example U.S. Patent Application Publications 2013/0090374, and 2012/0309050, and U.S. Pat. Nos. 7,858,367, 11,020,443, and 11,041,171.

Pharmaceutical compositions containing the expression vector may also be formulated for several other routes or modes of administration, including for example, oral (including both liquid and solid dosage forms), inhalation (via a pressurized propellant, topical (e.g., lotion, cream, gel, ointment, stick, spray and paste), and mucosal (e.g., vaginal).

The compositions may be provided in unit-dose or multi-dose sealed containers, such as ampoules and vials.

### Methods of Treatment

The present methods entail treatment of a subject having motor impairment due to loss of muscle function or due to neuron-muscle dysfunction at the level of the motor neurons. As understood in the art, “treatment” is an approach for obtaining beneficial or desired results, including clinical results. Such results may include one or more of alleviation or amelioration of one or more symptoms of motor impairment, diminishment of extent of the disease or disorder, stabilization of the state of the disease or disorder, delay or slowing of the disease or disorder, amelioration or palliation of the disease or disorder, and remission of the disease or disorder (whether partial or total), whether detectable or undetectable. As used herein, the term “subject” includes humans and non-human animals (e.g., non-human primates, livestock animals, domestic pets, and laboratory animals such as rodents). The term “motor impairment” as used herein refers to the loss of movement capability due to loss of muscle function. The loss of function is mainly due to muscle weakness, muscle atrophy, loss of motoneuron functionality, muscle contractibility impairment, and other muscle cell disorders (e.g., metabolic), spinal cord injury, motor neuron impairment, or motor neuron disease. Examples of motor impairment include total or partial paralysis (e.g., inability to walk or stand, inability to talk, to swallow and finally inability to breath (i.e., respiratory insufficiency), the latter leading to death), lack of muscle control, poor stamina, muscle cramps, spasticity, muscle weakness, and muscle atrophy.

Motor impairment, according to the International Neuromodulation Society, involves the partial or total loss of function of a body part, usually a limb or limbs. Diseases causing motor impairment may result in muscle weakness, poor stamina (i.e., fatigue), lack of muscle control, or total paralysis. Motor impairment is a major cause of physical disability. It is broadly caused by peripheral problems affecting muscles, problems in the central nervous system affecting output to muscles, and sensory problems affecting muscles, movement and balance. Motor impairment is often evident in neuromuscular diseases and in neurological conditions but it can also result from cancers of the central and the peripheral nervous system, or even in traumatic injuries.

Many neuromuscular and muscular diseases and disorders, including motor neuron diseases, such as certain neurodegenerative diseases, may manifest in one or more symptoms of motor impairment. Diseases and disorders characterized by or which manifest in one or symptoms of motor impairment, include neuromuscular diseases. The term “neuromuscular disease,” as used herein, includes any disease affecting motor neurons in the spinal cord or central nervous system (CNS), the peripheral nervous system (PNS), the neuromuscular junction, or skeletal muscle, all of which are components of the motor unit, thus ultimately affecting the movement ability of the subject. Damage to motor neurons in the spinal cord, the CNS, the PNS, the neuromuscular junction, or the skeletal muscle can cause muscle atrophy and weakness. Issues with sensation can also occur. Neuromuscular diseases can be acquired or genetic. Mutations of more than 500 genes have shown to be causes of neuromuscular diseases. Other causes include nerve or muscle degeneration, autoimmunity, toxins, medications, malnutrition, metabolic derangements, hormone imbalances, infection, nerve compression/entrapment, comprised blood supply, and trauma.

Representative examples of diseases or disorders characterized by motor impairment include Amyotrophic lateral sclerosis (ALS) including the more common or sporadic form (sALS) and the familial form (fALS), Charcot-Marie-Tooth disease, Multiple sclerosis, Muscular dystrophy, Duchenne and Becker muscular dystrophy, Myasthenia gravis, Myopathy, Myositis, including polymyositis and dermatomyositis, Peripheral neuropathy, Neuromyotonia, Lambert-Eaton disease, Friedreich's ataxia, traumatic nerve injury, diabetic neuropathy, Motor skills disorder, and Spinal Muscular Atrophy (SMA), spinal cord injuries, peripheral nerve injuries or traumatic nerve injuries, and muscle metabolic diseases. Yet other diseases and disorders that may be amenable to treatment with the disclosed expression vectors include hereditary myopathy, toxic neuropathy, autoimmune peripheral polyneuropathy, acute inflammatory demyelinating polyradiculoneuropathy (AIDP), chronic inflammatory demyelinating polyradiculoneuropahty (CIDP), vasculitic mononeuritis multiplex, paraneuropathy, idiopathic ganglionitis, amyotrophic lateral sclerosis, multifocal motor conduction lock neuropathy, or lower motor neuron syndrome, neuromuscular disease, muscular atrophy, drug-induced myopathy, sarcopenia, cachexia, type II muscle fiber atrophy, age-related muscular atrophy and acquired autoimmune primary muscle disorders.

Some diseases and disorders that may be amenable to treatment with the disclosed gene constructs and expression vectors include diseases and disorders that are classified as motor neuron diseases (MNDs), which are a group of rare neurodegenerative disorders that selectively affect motor neurons, the cells which control voluntary muscles of the body. Examples of MND include ALS, progressive bulbar palsy (PBP), pseudobulbar palsy, progressive muscular atrophy (PMA), primary lateral sclerosis (PLS), spinal muscular atrophy (SMA), and monomelic amyotrophy (MMA), as well as some rarer variants resembling ALS.

In some embodiments, the methods entail treatment of a subject with motor impairment in ALS, including motor impairment in sALS, including motor impairment in fALS, and including motor impairment in jALS.

In some embodiments, the methods may be “preventative” or “prophylactic” in that the expression vector (or transformed cells as per the ex vivo embodiments) is administered to a subject prior to manifestation of motor impairment. These embodiments might be particularly advantageous in situations where the subject is suspected of having an early stage of a neuromuscular disorder and/or has received a preliminary positive diagnosis. been tested or otherwise is believed to have (e.g., by way of family medical history) a predisposition to a disease or disorder (e.g., ALS). Preventative or prophylactic treatment may result in delay of onset of motor impairment, or reduced severity of motor impairment once it becomes manifest. Tests to determine motor impairment predisposition may include, for example, genetic tests, muscle biopsy, and electromyography, as known in the art. For example, fALS may be diagnosed by a genetic test.

### Modes of Administration

In some embodiments the pharmaceutical composition is administered to a subject parenterally (e.g., via intrathecal, subcutaneous, intravenous, intraventricular, intramuscular, or intraarterial injection, either bolus or infusion which may be continuous or non-continuous). Other routes/modes of administration include any medically acceptable route, representative examples of which may include oral, inhalation, topical, and mucosal.

In some embodiments, methods of treatment may entail cellular administration of the expression vector. In some embodiments, methods include isolating cells from a subject in need of treatment, also referred herein as “isolated cells”, placing the isolated cells in a suitable ex vivo culture system, exposing the isolated cells to an expression vector with a gene construct encoding a mammalian s-KL, and administering the isolated cells back into the subject. In some embodiments, the method includes optionally differentiating the isolated cells into a cell type with a differentiation agent. In some embodiments, the method includes optionally enriching the isolated cells before or after exposing the isolated cells to the expression vector. The cells isolated from the subject may be muscle cells, neuronal cells, or iPSCs. In some embodiments, the neuronal cells are motor neurons.

In some embodiments, the isolated cells are pluripotent stem cells which are or have been induced into pluripotency (e.g., iPSCs). The PSCs may be obtained from the subject (e.g., from fat tissue) in which case they may be reprogrammed to a pluripotent state (autologous). Alternatively, iPSCs may be obtained from a suitable cell bank (allogeneic), e.g., an umbilical cord cell bank. Pluripotency induction may be performed by any suitable means as known in the art. Generally, pluripotency induction involves modulation of specific cellular pathways, either directly or indirectly, in the isolated cells with pluripotency factors, which may be nucleic acid sequences, polypeptides, small molecules, or a combination thereof. In one embodiment the pluripotency factor is a polypeptide transcription factor or a polynucleotide encoding a transcription factor. Representative examples of pluripotency factor transcription factors include Oct-3/4, Cdx-2, Gbx2, Gsh1, HesX1, HoxA10, HoxA11, HoxB1, Irx2, Isl1, Meis1, Meox2, Nanog, Nkx2.2, Onecut, Otx1, Oxt2, Pax5, Pax6, Pdx1, Tcf1, Tcf2, Zfhx1b, Klf-4, Atbf1, Esrrb, Gcnf, Jarid2, Jmjd1a, Jmjd2c, Klf-3, Klf-5, Mel-18, Myst3, Nac1, REST, Rex-1, Rybp, Sall4, Sall1, Tif1, YY1, Zeb2, Zfp281, Zfp57, Zic3, Coup-Tf1, Coup-Tf2, Bmi1, Rnf2, Mta1, Pias1, Pias2, Pias3, Piasy, Sox2, Lef1, Sox15, Sox6, Tcf-7, Tcf711, c-Myc, L-Myc, N-Myc, Hand1, Mad1, Mad3, Mad4, Mxi1, Myf5, Neurog2, Ngn3, Olig2, Tcf3, Tcf4, Foxc1, Foxd3, BAF155, C/EBPβ, mafa, Eomes, Tbx-3; Rfx4, Stat3, Stella, and UTF-1. In one embodiment, a pluripotency factor includes nucleic acid sequences encoding the transcription factors Oct4, Sox2, Klf4, c-Myc, and Nanog.

iPSCs may be caused to differentiate into muscle cells or neuronal cells with a differentiation agent, as known in the art. Representative examples of differentiation agents for the differentiation of iPSCs into muscle cells include TGF-β, all-trans retinoic acid, dibutyryl-cyclic adenosine monophosphate (cAMP), platelet-derived growth factor-BB (PDGF-BB), and combinations thereof. Representative examples of differentiation agents for the differentiation of iPSCs into neuronal cells include retinoic acid, bone morphogenetic protein 4 (BMP4) nerve growth factor (NGF), retinoic acid receptor (RAR) agonists (e.g., TTNPB), glycogen synthase kinase 3 inhibitors (e.g., CHIR99021), Neurotrophin-3 (NT-3), and combinations thereof. Additional enrichment or selection may be performed on treated and/or differentiated cells. For example, in some embodiments, CD34+ cells are selectively enriched to isolate smooth muscle cells. In some embodiments, the iPSCs are differentiated and selectively enriched to isolate striated muscle cells. In some embodiments, neuronal cells are enriched by selecting Forkhead box A2 (FOXA2, also known as HNF3β, and TCF-3B) positive cells. In some embodiments, neuronal cells are enriched by selecting CD133 positive cells.

In other embodiments, muscle cells and/or neuronal cells are isolated from the subject. In some embodiments, myoblasts, and satellite cells (in either quiescent and activated states) are isolated from a subject by surgical methods (e.g., muscle biopsies) and isolated fluorescence-activated cell sorting (isolating, for example, Pax7 positive cells). In some embodiments, induced pluripotent stem cells, mesangioblasts, immortalized muscle precursor cells, or other multipotent cell lines are obtained and administered to the subject either locally or systemically.

Methods of introducing expression vectors into cells are well known in the art. Persons skilled in the art would readily appreciate the specific method depending on the nature of the vector. In some embodiments, the delivering or integrating may comprise transfecting, infecting, or transducing the cell with the expression vector. In some embodiments, the expression vector comprising the expression vector is delivered to a muscle cell by lipofection. Lipofection is described, for example, in U.S. Pat. Nos. 5,049,386, 4,946,787; and 4,897,355. Electroporation may be advantageous for plasmid vectors. In some embodiments, Factor H is increased during treatment, for example, by harvesting adipose tissue from the subject, purifying stem cells from the adipose tissue, treating the stem cells to increase secretion of Factor H (e.g., a small molecule compound, optionally Selegeline), and administering the treated stem cells to the subject. Adipose tissue harvesting and treatment is described, for example, in U.S. Patent Publication No. 2016/0193251.

The cells are administered to the subject after the cells are transformed with the gene constructs and any optional differentiation and/or enrichment is complete. Administration of these cells may be achieved by any suitable means as known in the art and disclosed elsewhere herein.

### Therapeutically Effective Dosage Amounts

The dose administered to a subject, particularly a human, in the context of the present disclosure, is “therapeutically effective” in the sense that it should be sufficient to achieve a beneficial or desired result, including a clinical result, as described above, in the subject over a reasonable time frame. Dosage will depend on a variety of factors including the strength of the specific expression vector employed, the condition of the subject, and the body weight of the subject, as well as the severity of the motor impairment. The size of the dose will also be determined by the existence, nature, and extent of any adverse side-effects that might accompany the administration of the expression vector.

Unit dosage forms of the pharmaceutical composition containing the expression vector may be formulated. The term “unit dosage form,” as used herein, refers to physically discrete units suitable as unitary dosages for human and animal subjects, each unit containing a predetermined quantity of the expression vector, calculated in an amount sufficient to produce the desired effect. The unit dosage form may be administered in any suitable volume of liquid over a suitable infusion or injection required by an embodiment. Doses may be determined by unit body weight (e.g., dose per subject kg), by body surface area (BSA), often denoted in squared meter BSA, or any suitable measurement of a subject.

In some embodiments utilizing a viral expression vector, the viral vector is delivered in a dose between 1×10⁶ to 5×10″ vector genomes per kg body weight (vg/kg) of the subject. The term “vector genome” (or “vg”) as used herein refers to the nucleic acid sequence that makes up a viral vector's genes plus any transgene encoded therein (e.g., mammalian s-KL).

In some embodiments, the viral vector is a lentivirus and is dosed between 1×10⁸ to 5×10¹² transducing unit per kg (TU/kg) per dose (e.g., one intravenous infusion).

In some embodiments, the expression vector is administered to a subject at a titer of from at least about 1×10⁵ viral genomes/mL to at least about 100×10¹⁶ viral genomes/mL. The terms “viral genomes” (vg) (also known as “genome equivalents”, “genome copies” (gc) or “genome particles” (gp)) as used in reference to a viral titer, refer to the number of virions containing an expression vector e.g., a recombinant AAV, regardless of infectivity or functionality.

In some embodiments, the viral vector delivering the s-KL gene construct is dosed in humans at from 1×10⁵ viral genomes to 5×10¹⁴ viral genomes in a suitable volume in order to achieve blood and tissue levels of the effective amount viral genomes is a s-KL genome dose that achieves between 200-1300 pg/ml of s-KL protein or polypeptide in the blood, CSF, or a desired target tissue like muscle. In some embodiments, the viral vector is dosed at 1×10⁶ vg or more in a deliverable volume in a single vial. In some embodiments, an initial dose of 1×10⁹ viral genomes is administered, and subsequent doses of 1×10¹¹ vg are administered.

In some embodiments, the effective amount is a dose that achieves between 1-2,000 pg/ml of polypeptide in the blood, CSF, or a desired target tissue. In some embodiments, the effective amount is a dose that achieves between 200-1300 pg/ml of polypeptide in the blood, CSF, or a desired target tissue. In some embodiments, the effective amount is a dose that achieves an average of about 550 pg/ml in circulation of an adult subject or within a range of 200 above or below that average. In some embodiments, the effective amount is a dose that achieves an average of about 950 pg/ml in circulation of a child subject or within a range of 300 above or below that average.

In some embodiments, the therapeutically effective dose results in a fold increase of gene expression of the nucleic acid sequence encoding the mammalian s-KL or a functional variant thereof. In some embodiments, the effective dose results in at least a two-fold increase of the expression. In some embodiments, the effective dose results in at least three-fold, or at least four-fold, or at least five-fold, or at least six-fold, or at least eight-fold, or at least 10-fold increase of gene expression.

The number of times a composition is administered to a subject in need thereof may depend on any one of more of numerous factors, and at the discretion of a medical professional, including the disease or disorder and its severity, and the subject's response to the formulation. Administration of a therapeutically effective amount of the expression vector occurs at least once. In other embodiments, administration occurs multiple times e.g., 2, 3, 4, 5, 6, 7, 8, 9, 10 or more times, in a given period, in order to achieve the desired therapeutic effect. The dosage of each administration and/or frequency of administrations may be adjusted as necessary based on the subject's condition and physiological responses. In the case wherein the subject's condition does not improve, upon the doctor's discretion the composition may be administered chronically, that is, for an extended period of time, including throughout the duration of the subject's life in order to ameliorate or otherwise control or limit the symptoms of the subject's disease or condition. In some embodiments, e.g., wherein the subject's status does improve, upon the doctor's discretion the composition may administered continuously.

In some embodiments, treatment may be administered over a period of about an hour. In some embodiments, treatment may be administered over a period of about an hour to about 24 hours in a day. In some embodiments, treatment may be administered 24 hours a day for multiple days, including by way of example only, 1 day, 2 days, 3 days, 4 days, 5 days, 6 days, 7 days, 10 days, 15 days, and 20 days. In some other embodiments, treatment may be temporarily reduced or temporarily suspended for a certain length of time (i.e., an “off period”). The length of the off period may vary widely, such as between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, and 365 days. In some other embodiments, treatment may be temporarily reduced for a certain length of time. The dose reduction during a reduction period may be from 10% to less than 100%, including by way of example only 10%, 15%, 20%, 25%, 30%, 35%, 40%, 45%, 50%, 55%, 60%, 65%, 70%, 75%, 80%, 85%, 90%, and 95%.

### Combination Therapies

Methods of treatment may include co-administration (i.e., administration within the same treatment period) of another active agent known to be effective in treating motor impairment (e.g., associated with neuromuscular diseases and disorders). Representative additional active agents include riluzole and edavarone, and combinations thereof. In some embodiments, methods of treatment are used in combination with the direct infusion of recombinant protein or gene therapy expressing IGF-1, TDP-43 (TAR DNA binding protein 43), EEAT2 (excitatory amino acid transporter 2), GDNF (Glial derived neurotrophic factor), Cardiotrophin-1, Brain-derived neurotrophic factor (BDNF), Ciliary neurotrophic factor (CNTF), Follistatin 344 (FSTN-344), and Factor H.

While the invention has been described in conjunction with the above aspects and embodiments, the foregoing description and the following examples are intended to illustrate and not limit the scope of the invention. Other aspects, advantages, and modifications within the scope of the invention will be apparent to those skilled in the art to which the invention pertains.

### Examples

### Materials and Methods

Animal Housing. The mouse model for this study was generated according (SOD1^{G93A}, also abbreviated SOD1) Mouse Model of ALS. Animals had free access to food and water and were kept under standard temperature conditions (22±2° C.) and a 12-h light/dark cycle (300 lux/0 lux).

Transgenic mice. Transgenic mice with the G93A human SOD1 mutation (C57bl6-Tg[SOD1-G93A]1Gur) were obtained from the Jackson Laboratory (Bar Harbor, ME, USA). Hemizygotes C57bl6 SOD 1^{G93A} males were obtained by crossing with C57bl6 females. The offspring was identified by PCR of DNA extracted from tail tissue. Experimental procedures were approved by the Ethics Committee of the Universitat Autònoma de Barcelona. The following experimental groups of mice were used: SOD1^{G93A} injected with AAV8-hDes-s-KL (SEQ ID NO: 7) or AAV8-Mock (SEQ ID NO: 8), WT littermates treated with AAV8-hDes-s-KL or AAV8-Mock (n=15-19/condition, 7-10 per each gender). To compensate the experimental groups, we allocated the animals were allocated according to weight and litter of origin. Animals were weighted every 4 weeks to monitor their overall health condition.

TABLE B
Sequence legend for SEQ ID NO: 8:

| Feature | Location | Size |
|---|---|---|
| Human Desmin promoter | 249 . . . 1250 | 1002 bp |
| Stuffer non-coding sequence | 1250 . . . 2465 | 1216 bp |
| WPRE | 2499 . . . 3093 | 595 bp |
| SV40 polyA | 3239 . . . 3360 | 122 bp |

Virus production and injection. Mouse s-KL cDNA was cloned between AAV2 ITRs under the regulation of the human desmin promoter. AAV8 viral stocks were produced by triple transfection into HEK293-AAV cells of the expression plasmids, Rep8Cap2 plasmids containing AAV genes and pXX6 plasmid containing adenoviral genes needed as helper virus. The AAV2 ITR encoding plasmid is described in Piedra et al., Hum. Gene Ther. Methods 26 (1):35-42 (2015). The pXX6 encoding plasmid is describe in Xiao et al., J. Virol. 72 (3):2224-2232 (1998). The Rep8Cap2 encoding plasmid is described in Gao et al., Proc. Natl. Acad. Sci. U.S.A. 99 (18):11854-11859 (2002).

AAV particles were purified by iodixanol gradient. Titration was evaluated by picogreen (Invitrogen) quantification and calculated as viral genomes per milliliter (vg/ml). Control serotype-matching AAV empty vectors (mock) were used as control.

For intravenous administration, 1.8×10¹⁴ and 3×10¹⁴ vg/kg of AAV8-s-KL or AAV8-Mock in a total volume of 250 μl suspension were injected in the tail vein of 6-weeks old mice.

Real time PCR. For RNA extraction 1000 μl of Qiazol (Qiagen) were added, and tissue homogenized for 6 minutes with Tyssue Lyser LT (Qiagen) at 50 Hz twice. Then, samples were purified with chloroform, precipitated with isopropanol, washed with 70% ethanol and resuspended in 20 μl RNAse free water. The RNA concentration was measured using a NanoDrop ND-1000 (Thermo Scientific).

One μg of RNA was reverse-transcribed using 10 μmol/l DTT, 200 U M-MuLV reverse transcriptase (New England BioLabs), 10 u RNase Out Ribonuclease Inhibitor (Invitrogen), 1 μmol/l oligo(dT), and 1 μmol/l of random hexamers (BioLabs). The reverse transcription cycle conditions were 25° C. for 10 min, 42° C. for 1 h and 72° C. for 10 min. The mRNA expression of s-KL was analyzed by means of specific primer sets (s-KL-Fw: 5′-TCATAATGGAAACCTTAAAAGCAA-3′ (SEQ ID NO: 9) and s-KL-Rv: 5′-CACTGGGTTTTGTCAAAGGA-3′ (SEQ ID NO: 10), using the Taqman probe 5′-6FAM-3′BHQ-1 s-KL-Pr: 5′-AGAAGAGTCCTCGCCGGATGCTGTA-3′ (SEQ ID NO: 11). Mouse 36B4 was used to normalize the expression levels of the s-KL (m36b4-Fw (Rplp0): 5′-ATGGGTACAAGCGCGTCCTG-3′ (SEQ ID NO: 12); m36b4-Rv (Rplp0): 5′-AGCCGCAAATGCAGATGGATC-3′ (SEQ ID NO: 13); Probe 5-HEX-3′BHQ-1 m36B4-Pr (Rplp0): 5′-TGTGGAGACTGAGTACACCTTCCCA-3′ (SEQ ID NO: 14).

The thermal cycling conditions comprised 5 min polymerase activation at 95° C., 45 cycles of 15 s at 95° C., 30 s at 60° C., 30 s at 72° C. and 5 s at 65° C. to 95° C. (increasing 0.5° C. every 5 s). Fluorescence detection was performed at the end of the PCR extension, and melting curves were analyzed by monitoring the fluorescence of the Taqman probes.

Spinal Cord Organotypic Cultures. Postnatal day 8 Sprague-Dawley rats were euthanized, and the spinal cord was aseptically harvested and placed in ice-cold high glucose Gey's Balanced Salt Solution, where meninges were removed. The spinal cord was cut transversely in 350 mm thick slices using a chopper. L4-L5 lumbar sections were transferred to Millicell-CM porous membranes in plates containing incubation medium.

After the axotomy performed during the culture procedure a high number of neurons die naturally and glial cells show a strong reactivity, so cultures need to be left for one week to stabilize. Then, a one-microliter drop containing 10⁸ IU of Ad5-CMV-s-KL or Ad5-CMV-Null, or medium as a control, was added on top of each slice. In an alternative assay, adeno-associated viruses were used, namely AAV9-CMV-s-KL, AAV9-CMV-Null vectors, or medium as a control. Slices were maintained for another week to allow the transgene to be expressed. At 14 days in vitro (DIV) glutamic acid (50 μM) in Locke's solution was added to the plate for 30 minutes to induce acute excitotoxicity, which was then replaced by medium. At 19 DIV the slices were harvested and fixed with paraformaldehyde 4% for histological staining.

Electrophysiological tests. Motor nerve conduction tests were performed every 4 weeks from 8 to 16 weeks of age. The sciatic nerve was stimulated by single pulses (Grass S88 stimulator) delivered through needle electrodes placed at the sciatic notch. The evoked compound muscle action potential (CMAP) was recorded from gastrocnemius (GM) and plantar interossei (PL) muscles with microneedle electrodes. Electromyographic signals were amplified and displayed on a digital oscilloscope (Tektronix 450S), for measuring the amplitude and the latency of the CMAP.

Motor evoked potentials were evaluated to assess central motor pathways. Electrical stimuli of supramaximal intensity were delivered with needle electrodes placed subcutaneously over the skull overlaying the sensorimotor cortex, and the MEPs recorded from GM muscle using microneedle electrodes.

Locomotion tests. Motor coordination, strength and balance were evaluated by means of the Rotarod test in treated and untreated SOD1^{G93A} animals. Each mouse was placed three times in the rotarod turning at a constant speed of 14 rpm and the longest time until falling recorded. A maximum time of 180 s was set. The test was performed every other week from 8 to 20 weeks of age. Clinical disease onset for each mouse was determined as the first week when the maintenance time was lower than 180 s.

Histology. At 16 weeks of age, mice were transcardially perfused with 4% paraformaldehyde in PBS and the lumbar spinal cord, tibial nerve and gastrocnemius muscles were harvested.

For NMJ labeling, the GM muscles were cryopreserved in 30% sucrose in PBS and 60 μm longitudinal sections were serially cut with a cryotome and collected in sequential series of 10. Sections were blocked with PBS-Triton-FBS and incubated 48 h at 4° C. with primary antibodies anti-synaptophysin (1:500, AB130436, Abcam), anti-neurofilament 200 (NF200, 1:1000, AB5539, Millipore), and anti-5100β (1:1, 22520, Immunostar). After washes, sections were incubated overnight with Alexa 594-conjugated secondary antibody (1:200; Life Science) and Alexa 488 conjugated α-bungarotoxin (1:200, B-13422, Life technologies). The proportion of innervated endplates was determined by classifying each endplate as either occupied (when presynaptic terminals overly the endplate) or vacant (no presynaptic label in contact with the endplate). At least 4 fields totaling>100 endplates were analyzed per muscle. For collateral sprouting, the number of sprouts per endplate was counted as the neurofilament-positive projections from pre-synaptic terminal or pre-terminal nodes on confocal z projections. For analysis, we considered both the total number of sprouts and the proportion of occupied end plates that were innervated by a collateral sprout.

Data analysis. All experiments were performed by researchers blinded for the different treatments of each mouse group, and random allocation of animals in groups taking into account weight and litter. Data are expressed as mean±SEM. Electrophysiological and locomotion tests results were statistically analyzed using one-way or repeated measurements ANOVA with Tukey post-hoc test. For MEPs electrophysiological results Student's t test was applied. For clinical disease onset Log-rank (Mantel-Cox) test was applied. Histological and molecular biology data were analyzed using t-Student, one or 2-way ANOVA with Tukey or Holm-Sidák post-hoc tests.

### Results

### 1—Klotho is Downregulated in the SOD1^{G93} Mouse Model

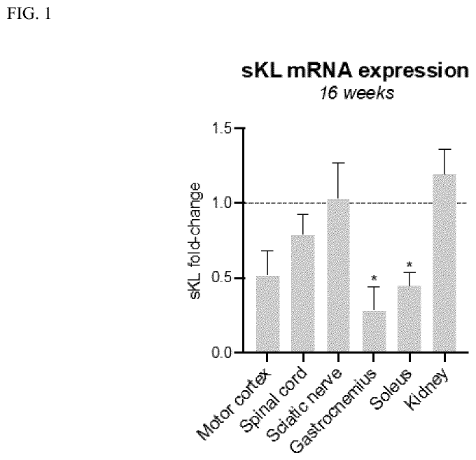

Once carried out the mRNA extraction of several tissues of the mouse model, it was observed that mRNA expression of secreted αKlotho (s-KL) was decreased in the motor cortex, spinal cord, gastrocnemius, and soleus of SOD1^{G93A} , being significant in the muscles analyzed. Expression ratios of s-KL relative to wild-type (WT) at 16 weeks, the end stage of the disease (n=7 SOD1, 3-10 WT mice per group, ***p<0.001, **p<0.01, *p<0.05). Data are shown in FIG. 1, wherein the s-KL mRNA expression (fold-change) is illustrated as mean±SEM in the several studied tissues.

### 2—In Vitro Assay. Klotho Protects Spinal Motoneurons from Excitotoxicity in Organotypic Cultures

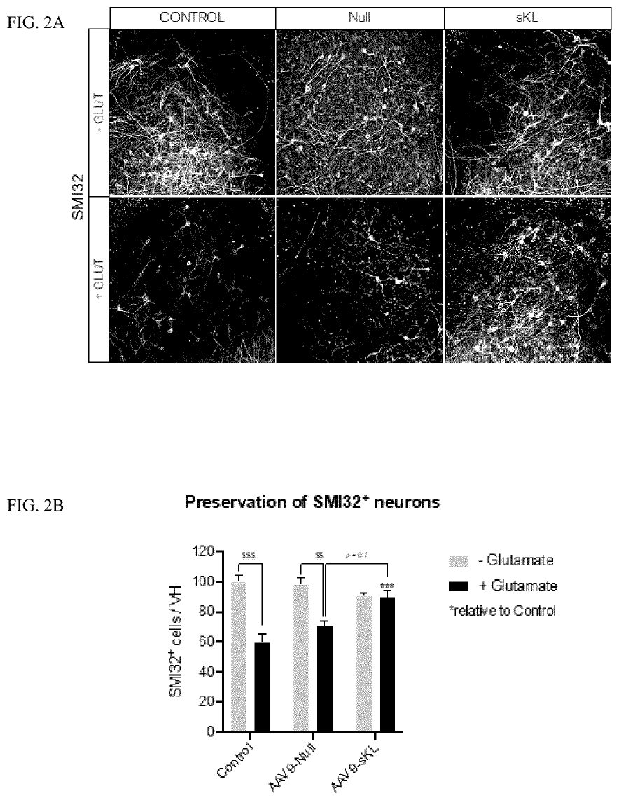

Rat spinal cord organotypic slices were treated with AAV9 coding for s-KL (SEQ ID NO: 15) or a null sequence (SEQ ID NO: 16) and exposed to glutamate. Data are shown in FIG. 2A, fluorescence microscopic image and FIG. 2B, bar diagram, in which second bar of each set correspond to glutamate-induced excitotoxicity (GLUT+). Overexpression of s-KL preserves neurons located at the ventral horn (VH) of the spinal cord from glutamate-induced excitotoxicity compared to non-treated controls or AAV9-Null. All data are the mean±SEM (n=6 per group, ***p<0.001, **p<0.01, *p<0.05).

Tables C and D list particular fragment sequences in each of SEQ ID NOs: 15 and 16, respectively.

TABLE C
Sequence legend for SEQ ID NO: 15

| Feature | Location | Size |
|---|---|---|
| CMV enhancer | 343 . . . 722 | 380 bp |
| CMV promoter | 723 . . . 934 | 212 bp |
| Secreted Klotho | 1017 . . . 2669 | 1653 bp |
| SV40 poly(A) signal | 2715 . . . 2836 | 122 bp |

TABLE D
Sequence legend for SEQ ID NO: 16

| Feature | Location | Size |
|---|---|---|
| CMV enhancer | 343 . . . 722 | 380 bp |
| CMV promoter | 723 . . . 934 | 212 bp |
| Stuffer non-coding sequence | 1295 . . . 3311 | 2017 bp |
| WPRE | 3347 . . . 3941 | 595 bp |
| SV40 poly(A) signal | 4003 . . . 4124 | 122 bp |

An equivalent assay with equivalent results was performed with rat spinal cord organotypic slices, which were treated with an adenoviral vector coding for sKL or a null sequence and exposed to glutamate (GLUT+) that induces a cytotoxic effect. Data of this alternative assay are not shown, but overexpression of s-KL preserved neuronal survival located at the ventral horn (VH) of the spinal cord from glutamate-induced excitotoxicity compared to non-treated controls (GLUT−) or Ad-Null, which showed high mortality.

### 3—Gene Therapy Strategy

SOD1 female mice were intravenously treated with 1.8×10¹⁴ vg/kg or 3×10¹⁴ vg/kg of AAV-hDesmin-s-KL-WPRE vectors (n=15) at 6 weeks of age. WT (n=10) and SOD1 female mice (n=15) were treated at the same dose with AAV-hDesmin-Null-WPRE as a control. The human desmin promoter was used to restrict expression to striated muscles. The WPRE sequence was used to stabilize the mRNA. Animals were followed-up weekly with Rotarod and grip strength tests, and at 8, 12 and 16 weeks by nerve conduction tests. Tissues were harvested at the end stage of the disease and processed for histological and expression analysis.

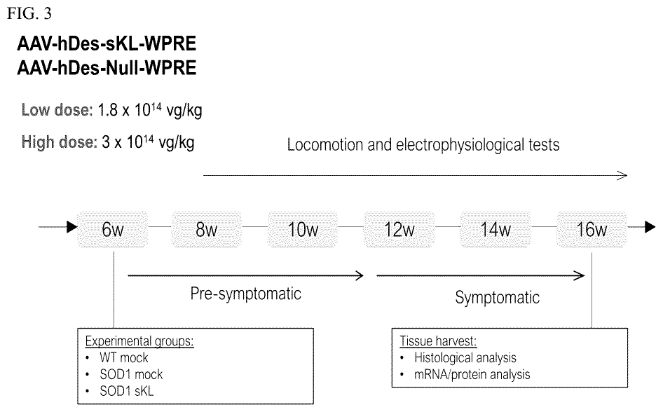

A schematic view of an embodiment gene therapy strategy is depicted in FIG. 3.

### 4—Improved Compound Muscle Action Potentials (CMAP)

After the gene therapy according to the previous section, motor nerve conduction tests were performed with two needle electrodes placed at the sciatic notch and stimulating the sciatic nerve at single pulses of 20 μs of duration. The compound muscle action potential (CMAP) was recorded from tibialis anterior (TA) and plantar interossei (PL) muscles with microneedle electrodes at 8, 12, and 16 weeks of age.

The treatment promoted a significant preservation of the CMAP amplitude of the tibialis anterior and plantar interossei muscles, indicating that s-KL overexpression promotes motor functional improvement of SOD1 ^{G93A} female mice.

These data show that the motor function is preserved, since motor neuron and the muscle cells well communicate.

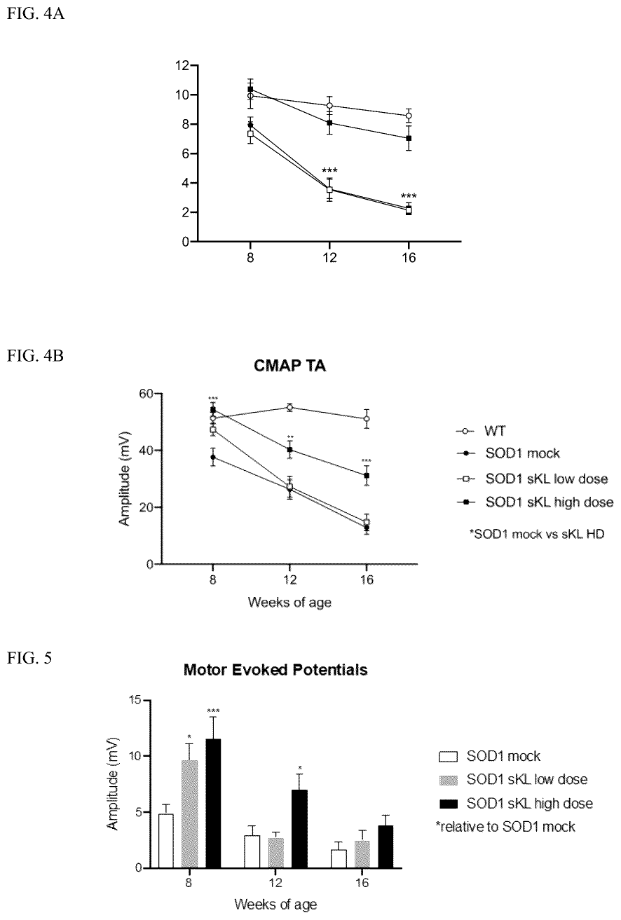

CMAP values (Amplitude in mV) are represented in FIG. 4A, Plantar muscles, PL and FIG. 4B, Tibialis anterior, TA for wild type (WT), SOD1 mock, SOD1 s-KL low dose, SOD1 s-KL high dose. (***p<0.001, **p<0.01, *p<0.05 SOD1 mock versus SOD1 high dose, mean±SEM).

### 5—Increased Motor Evoked Potentials Amplitude (MEPs)

To evaluate the central corticospinal descending pathways, motor evoked potentials (MEP) were recorded from TA muscles. The motor cortex was electrically stimulated with pulses of 0.1 ms of duration and at supramaximal intensity, delivered with needle electrodes placed subcutaneously over the skull. Gene therapy increased the amplitude of MEPs, indicating enhanced connection between upper and lower motoneurons (***p<0.001, **p<0.01, *p<0.05, relative to SOD1 mock, mean±SEM). Data are depicted in FIG. 5, where the first bar of the set is for the assay in SOD1 mock, the second bar is for SOD1 s-KL low dose, and the third bar is the SOD1 s-KL high dose.

### 6-Klotho Enhances Motor Function of SOD1^{G93A} Mice

The rotarod test was performed to evaluate motor coordination and balance of the animals. Mice were placed over a rotating rod a constant speed of 14 rpm; 180 s was chosen as the cut-off time. To test fore and hindlimb grip-strength mice were placed over a metallic grid and allowed the four paws to grasp the bars while being pulled away by the tail. During the test the instrument recorded the peak pull-force prior to release

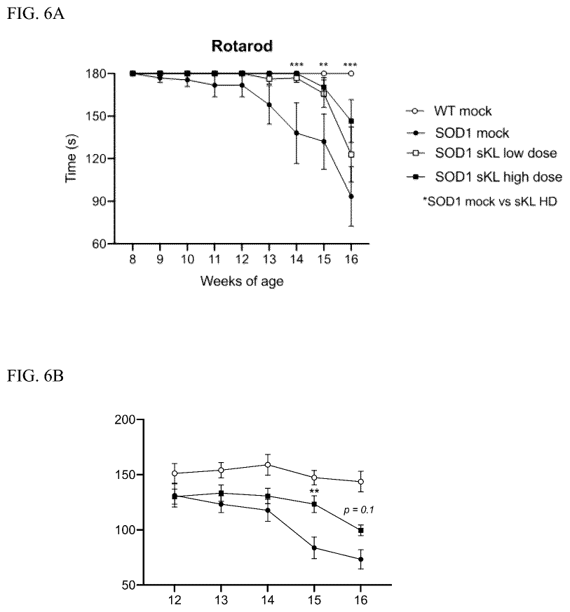

Overexpression and secretion of s-KL by muscles improved SOD1^{G93A} mice motor performance on the Rotarod compared to non-treated controls. Strength of the high dose treated mice was also improved at end stage as assessed by the grip strength test (***p<0.001, **p<0.01, *p<0.05, SOD1 high dose relative to SOD1 mock, mean±SEM). Data are depicted in FIG. 6A-FIG. 6B, for rotarod as Time (s) in FIG. 6A and for Grip strength as Force (g) in FIG. 6B.

### 7—Delayed Clinical Disease Onset

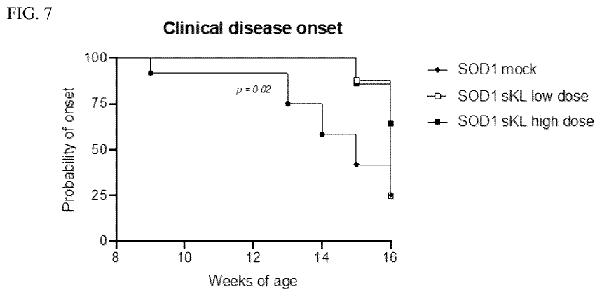

The clinical disease onset for each mouse of the previous sections was determined as the first week the animals could not be maintained on the Rotarod for 180 s. The treatment was able to significantly delay the disease onset and at 16 weeks only 35% of the high-dose-treated mice (treated with the AAV9-s-KL vector, SEQ ID NO: 15) showed motor deficits, compared to mock-treated mice (treated with the AAV9-null vector, SEQ ID NO: 16). The data depicted in FIG. 7 illustrate the probability of onset each mouse type.

### 8-s-KL Preserves Neuromuscular Junctions (NMJ) and Muscle Mass

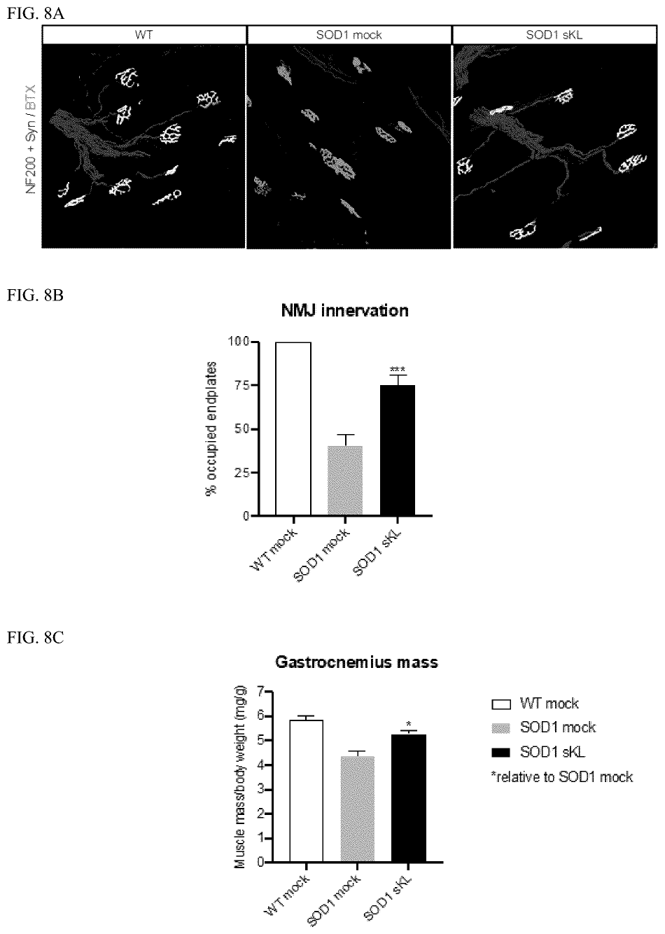

Gastrocnemius longitudinal sections were labeled for neurofilament 200 (NF200), anti-synaptophysin and alfa-bungarotoxin (BTX). Endplates were classified as occupied (when presynaptic terminals overlied or touched the endplate) or vacant (no presynaptic labeled in contact with the endplate). The secretion of s-KL by skeletal muscles significantly increased the proportion of NMJ occupied by presynaptic terminals and mitigated muscle wasting as evidenced by a greater muscle mass (***p<0.001, **p<0.01, *p<0.05, SOD1 mock vs. SOD1 high dose, data presented as mean±SEM). Data are depicted in FIG. 8A-FIG. 8C. FIG. 8A, a fluorescence microscope image, shows the sections (dark grey) labeled for NF200, and the sections (light grey) labeled for BTX. FIG. 8B indicates the percentage of occupied endplates in WT mice, and in mock (SOD1 mock) and treated (SOD1s-KL). FIG. 8C indicates the muscle mass per body weight (mg/g) of WT mice, and in mock (SOD1 mock) and treated (SOD1s-KL).

### 9—Secretion of s-KL by Muscles Protects Spinal Motor Neurons

At 16 weeks of age, mice were transcardially perfused with 4% paraformaldehyde and the lumbar spinal cord was harvested. For spinal MN evaluation, spinal cords were postfixed during 4 h, cryopreserved in 30% sucrose in PBS, and 20 μm transverse sections were serially cut using a cryotome. One out of every two slides from the lumbar spinal region comprising L4 to L6 of each animal was stained with cresyl violet. Motoneurons were identified by their localization in the ventral horn and following strict size and morphological criteria.

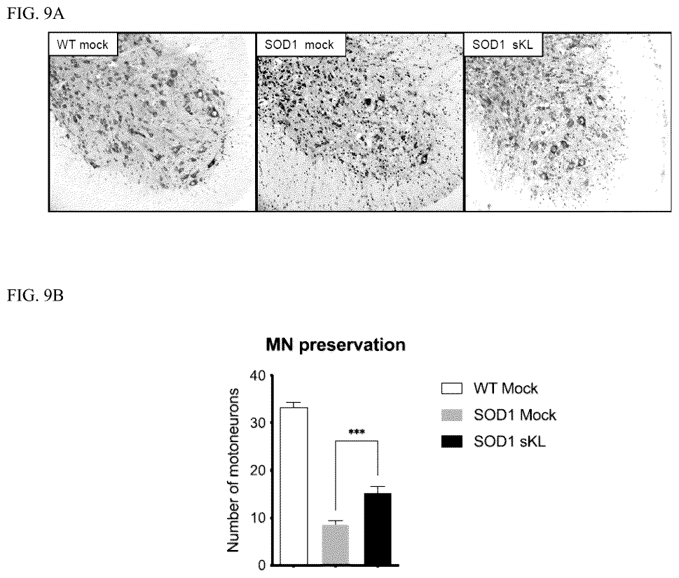

Skeletal muscle secreted s-KL protected motoneurons in the lumbar spinal cord, which is the region that undergoes more motoneuron death in the SOD1 mouse model (n=8/group, two-way ANOVA test, ***p<0.001; data presented as mean±SEM). Dare are depicted in FIG. 9A-FIG. 9B. FIG. 9A, a white light microscope image, shows the sections (dark grey) labeled for cresyl violet, which stains the Nissl substance in the neurons and cell nuclei, thereby revealing neuronal structure. FIG. 9B quantifies the neuronal structure in WT mice (WT Mock), and mock (SOD1 mock) and treated (SOD1 sKL) mice.

### 10—Klotho Protects the Spinal Cord from Neuroinflammatory Insults in ALS

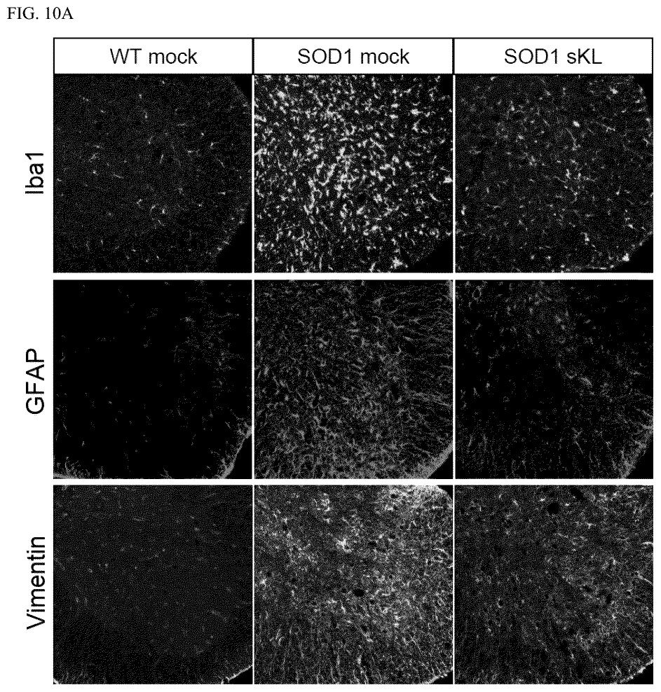

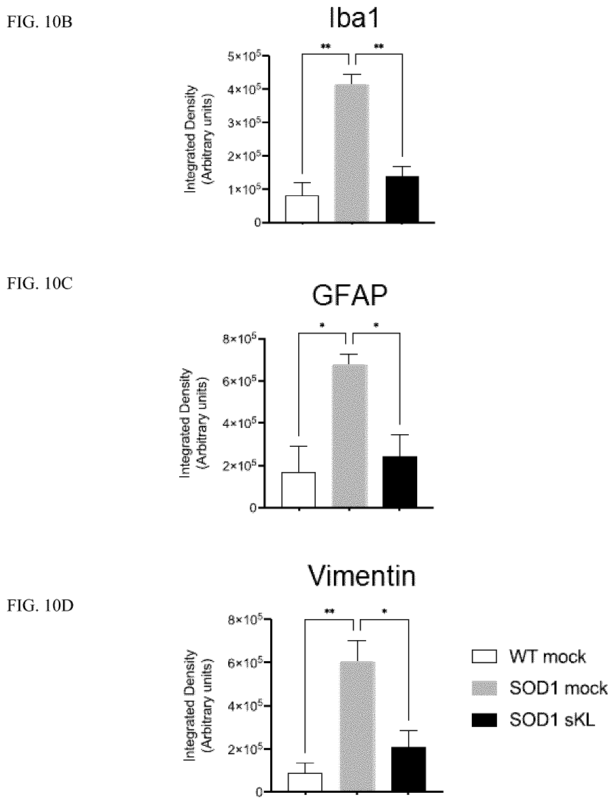

Delivery of s-KL by the muscles reduced microglia reactivity in the lumbar spinal cord of treated SOD 1^{G93A} mice as assessed by Iba1 fluorescence. Astrocytosis was analyzed by GFAP and Vimentin labeling and was corrected close to WT levels (n=8/group, two-way ANOVA test, **p<0.01, *p<0.05). Data are expressed as mean±SEM. Data are depicted in FIG. 10A-FIG. 10D. FIG. 10A, a fluorescence microscope image, shows the sections (top row) labeled for lba1, the sections (middle row) labeled for GFAP, and the sections (bottom row) labeled for Vimentin. FIG. 10B-FIG. 10D show the quantification of staining in FIG. 10A for WT mice (WT Mock), and mock (SOD1 mock) and treated (SOD1 sKL), illustrating decreased staining for Iba1, GFAP and Vimentin after s-KL treatment. FIG. 10B indicates the integrated density (in arbitrary units) of lba1-labeled images. FIG. 10C indicates the integrated density (in arbitrary units) of GFAP-labeled images. FIG. 10D indicates the integrated density (in arbitrary units) of Vimentin-labeled images.

### 11—Gene Therapy Strategy With AAVmyo Vectors

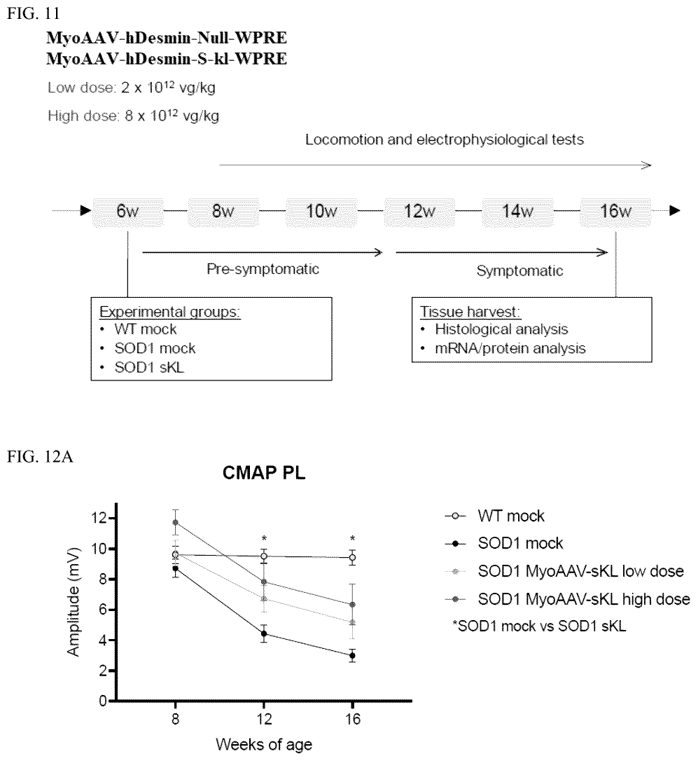

AAVmyo is an AAV serotype (Tabebordbar et al., Cell 184 (19):4919-4938 (2021)) that has a tropism for striated muscles with a superior transduction efficiency in mice and non-human primates. To decrease the dose of AAV vectors administered and avoid possible side-effects, the AAVmyo serotype was tested for s-KL gene therapy. A schematic view of an embodiment of a gene therapy strategy is depicted in FIG. 11.

Six-week-old SOD1 mice were intravenously treated with 2×10¹² vg/kg (n=6; AAVmyo-s-KL low dose) or 8×10¹² vg/kg (n=8; AAVmyo-s-KL high dose) of AAVmyo-hDesmin-sKL-WPRE expression vectors. The AAVmyo-s-KL high dose and AAVmyo-sKL-low dose were 150-fold and 37.5-fold lower, respectively, as compared to the doses used with the gene expression experiments using AAV8 expression vectors in FIG. 3. WT (n=14) and SOD1 female mice (n=18) were treated at the same dose with AAVmyo-hDesmin-Null-WPRE as a control. Animals were followed-up as in previous studies and euthanized at 16 weeks of age for tissue harvest.

### 12—AAVmyo-Des-sKL Improves Compound Muscle Action Potentials (CMAP)

The treatment with AAVmyo-Des-sKL at the higher dose showed significant preservation of the plantar (PL) and GM compound muscle action potential (CMAP) amplitude from weeks 12 and 8, respectively, in treated SOD1^{G93A} mice when compared to control SOD1^{G93A} mice. With this novel serotype, similar levels of neuromuscular connectivity preservation to that of AAV8-Des-sKL were achieved, while reducing the viral titer administered by 37.5-fold (two-way ANOVA test, *p<0.05; *SOD1 mock versus SOD1 sKL high dose; data are expressed as mean±SEM).

Data are depicted in FIG. 11A-FIG. 11B. CMAP values (Amplitude in mV) are represented in FIG. 11A, Plantar muscles, PL and FIG. 11B, Tibialis anterior, TA for wild WT mock, SOD1 mock, SOD1 Myo-AAV-s-KL low dose, SOD1 Myo-AAV-s-KL high dose. *p<0.05 SOD1 mock versus SOD1 Myo-AAV-s-KL, mean±SEM).

### 13-AAVmyo-Des-sKL Increases Motor Evoked Potentials Amplitude (MEPs)

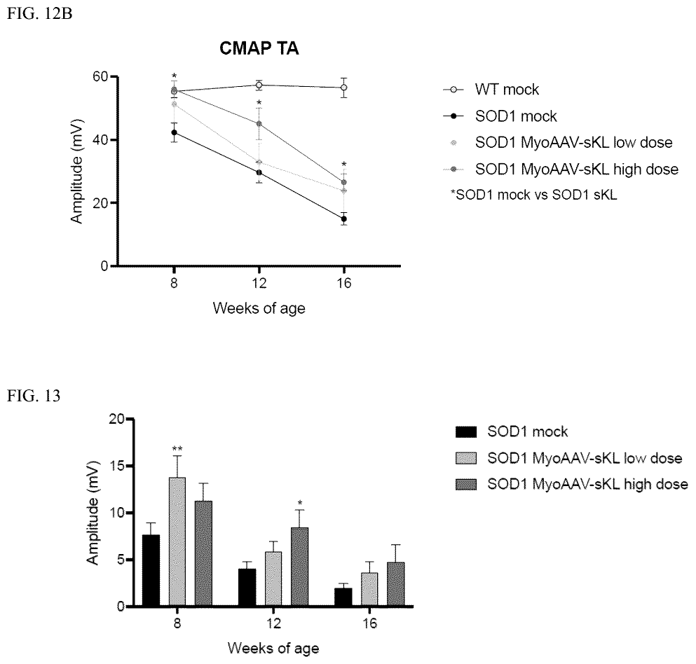

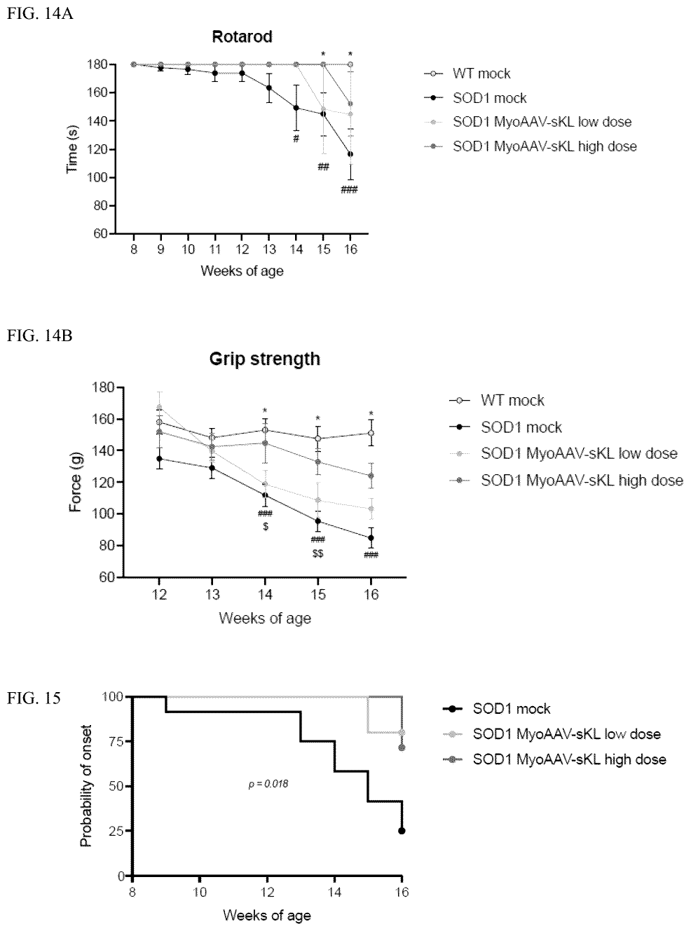

The amplitude of motor evoked potentials was higher in treated SOD1^{G93A} mice compared to SOD1-mock mice, also indicating a higher preservation of central motor pathways. Compared to the AAV8-DessKL treatment, the AAVmyo-Des-sKL treatment preserved MEP amplitude to a greater extent (two-way ANOVA test, **p<0.01, *p<0.05; *SOD1 mock versus SOD1 sKL high dose; data are expressed as mean±SEM). Data are depicted in FIG. 12, where the first bar of the set is for the assay in SOD1 mock, the second bar is for SOD1 AAVmyo-s-KL low dose, and the third bar is the SOD1 AAVmyo-s-KL high dose.

### 14-Klotho Enhances Motor Function of SOD1^{G93A} Mice

A slower progression of the locomotion decline was observed in SOD1 mice treated with AAVmyo-Des-sKL. The force of the animals was also preserved, especially in the high dose treated group, were the SOD1 mice performed better than the animals injected with AAV8-DessKL (two-way ANOVA test, ***p<0.001, **p<0.01, *p<0.05; *SOD1 mock versus SOD1 sKL high dose, # SOD1 mock versus WT mock; mean±SEM). Data are depicted in FIG. 13A-FIG. 13B, for rotarod as Time (s) in FIG. 13A and for Grip strength as Force (g) in FIG. 13B.

### 15-Delayed Clinical Disease Onset

The clinical disease onset was significantly delayed in both groups of AAVmyo-Des-sKL-treated SOD1 mice, similar to mice treated with the high dose of AAV8-Des-sKL (Log-rank Mantel-Cox test). Data are depicted in FIG. 14, which illustrates the probability of onset each mouse type.

### Other Publications Incorporated Herein by Reference

Zeldich et al., “Klotho Is Neuroprotective in the Superoxide Dismutase (SOD1^{G93A}) Mouse Model of ALS” J. Mol. Neurosci. 69 (2):264-285 (2019)
Minamizaki et al., “Soluble Klotho causes hypomineralization in Klotho-deficient mice” J. Endocrinol. 237 (3):285-300 (2018)
WO2017085317 (Universitat Autònoma de Barcelona et al.)

All patent publications and non-patent publications are indicative of the level of skill of those skilled in the art to which this disclosure pertains. All these publications (including any specific portions thereof that are referenced) are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A gene construct comprising a nucleic acid comprising a first promoter operatively linked to a nucleic acid sequence encoding a mammalian s-KL or a functional variant thereof, wherein the first promoter is a muscle-cell specific promoter.

2. The gene construct of claim 1, wherein the mammalian s-KL encoded by the nucleic acid sequence is a human s-KL having the amino acid sequence of SED ID NO: 1.

3. The gene construct of claim 2, wherein the nucleic acid sequence SEQ ID NO: 3.

4. The gene construct of claim 1, wherein the mammalian s-KL encoded by the nucleic acid sequence is a murine s-KL having the amino acid sequence of SEQ ID NO: 2.

5. The gene construct of claim 4, wherein the nucleic acid sequence is SEQ ID NO: 4.

6. The gene construct of claim 1, wherein the functional variant has at least 85% amino acid sequence identity to SEQ ID NO: 1.

7. The gene construct of claim 1, wherein the functional variant has at least 88% amino acid sequence identity to SEQ ID NO: 1.

8. The gene construct of claim 1, wherein the functional variant has at least 95% amino acid sequence identity to SEQ ID NO: 1.

9. The gene construct of claim 1, wherein the functional variant has at least 98% amino acid sequence identity to SEQ ID NO: 1.

10. The gene construct of claim 1, wherein the first promoter is a mammalian desmin promoter.

11. The gene construct of claim 10, wherein the mammalian desmin promoter is a human desmin promoter.

12. The gene construct of claim 11, wherein the human desmin promoter has the nucleic acid sequence of SEQ ID NO: 5.

13. The gene construct of claim 1, further comprising a second promoter operatively linked to the nucleic acid sequence encoding the mammalian s-KL or a functional variant thereof; and wherein the first and second promoters are different.

14. The gene construct of claim 13, wherein the second promoter is a muscle cell-specific promoter; or wherein the second promoter is a neuronal cell-specific promoter.

15. The gene construct of claim 13, wherein the second promoter is a constitutive promoter.

16. The gene construct of claim 15, wherein the constitutive promoter is a cytomegalovirus (CMV) promoter.

17. The gene construct of claim 13, wherein the second promoter is an inducible promoter.

18. The gene construct of claim 17, wherein the inducible promoter is a zinc-driven metallothionein promoter.

19. A plasmid comprising the gene construct of claim 1, and an initiation sequence operatively linked to the first promoter.

20. An expression vector, comprising
(a) the gene construct of claim 1 or a plasmid comprising the gene construct and an initiation sequence operatively linked to the first promoter, wherein the expression vector may or may not have a muscle cell tropism; or
(b) a nucleic acid construct comprising a first promoter functional in a muscle cell, a neuronal cell, or an induced pluripotent stem cell (iPSC) operatively linked to a nucleic acid sequence encoding a mammalian s-KL or functional variant thereof, wherein the expression vector has a muscle cell tropism.

21. The expression vector of claim 20, which is a viral expression vector.

22. The expression vector of claim 21, which is an adeno-associated virus (AAV) vector of a serotype with muscle cell tropism; or which is an AAV vector is of a serotype with neuronal cell tropism.

23. The expression vector of claim 22, wherein the AAV vector is of a serotype with muscle cell tropism.

24. The expression vector of claim 23, wherein the AAV vector is an AAV1, AAV8, AAV9, or AAVmyo vector; or wherein the AAV vector comprises an AAV capsid polypeptide that comprises or consists of the amino acid sequences of any one of SEQ ID NOs: 17-29.

25. The expression vector of claim 24, wherein the AAV vector is a AAVmyo vector.

26. The expression vector of claim 24, wherein the AAV vector comprises an AAV capsid polypeptide that comprises or consists in the amino acid sequences of any one of SEQ ID Nos: 17-29.

27. The expression vector of claim 26, wherein the AAV vector comprises an AAV capsid polypeptide that comprises or consists in the amino acid sequence of SEQ ID NO: 17 or 26.

28. The expression vector of claim 24, wherein the AAV vector is an AAV9 vector.

29. The expression vector of claim 22, wherein the AAV vector is of a serotype with neuronal cell tropism.

30. The expression vector of claim 29, wherein the AAV vector is an AAV1 vector.

31. The expression vector of claim 29, wherein the AAV vector is an AAV8 vector.

32. The expression vector of claim 29, wherein the AAV vector is an AAV9 vector.

33. The expression vector of claim 20, which is a lipid-based vector.

34. The expression vector of claim 33, which is a lipid nanoparticle (LNP) or a liposome.

35. The expression vector of claim 20, further comprising at least one non-coding regulatory element.

36. The expression vector of claim 35, wherein the regulatory element is any one or more of a poly A sequence, a protein translation initiation site consensus nucleic acid sequence, a post-transcriptional regulatory element nucleic acid sequence, 5′ and 3′ inverted terminal repeat nucleic acid sequences, or an intron.

37. The expression vector of claim 36, wherein the poly A sequence is an SV40 Poly A sequence.

38. The expression vector of claim 37, wherein the post-transcriptional regulatory element nucleic acid sequence is a Hepatitis B Virus Postranscriptional Regulatory Element (HPRE) or a Woodchuck Hepatitis Postranscriptional Regulatory Element (WPRE).

39. A pharmaceutical composition comprising a plasmid comprising a gene construct comprising a nucleic acid comprising a first promoter operatively linked to a nucleic acid sequence encoding a mammalian s-KL or a functional variant thereof, wherein the first promoter is a muscle-cell specific promoter, or the-expression vector of claim 20 and a pharmaceutically acceptable carrier.

40. An isolated cell comprising the gene construct of claim 1 or a plasmid comprising the gene construct and an initiation sequence operatively linked to the first promoter; wherein the isolated cell is a human muscle cell, a human neuronal cell, or a human induced pluripotent stem cell (iPSC) that can be differentiated into a muscle cell or a neuronal cell.

41. The isolated cell of claim 40, which is a muscle cell.

42. The isolated cell of claim 41, which is a skeletal muscle cell.

43. The isolated cell of claim 41, which is a striated muscle cell.

44. The isolated cell of claim 40, which is a neuronal cell.

45. The isolated cell of claim 44, wherein the neuronal cell is a motor neuron.

46. The isolated cell of claim 40, which is an iPSC that can be differentiated into a muscle cell or a neuronal cell.

47. A cell therapy, comprising administering to a subject in need thereof the isolated cell of claim 40.

48. A method of treating motor impairment, comprising administering to a subject in need thereof the pharmaceutical composition of claim 39.

49. The method of claim 48, wherein the subject has a disease or disorder characterized by or which manifests in a symptom of motor impairment.

50. The method of claim 48, wherein the disease or disorder is a neuromuscular disease or disorder.

51. The method of claim 50, wherein the neuromuscular disease is Amyotrophic lateral sclerosis (ALS).

52. The method of claim 51, wherein the ALS is sporadic ALS (sALS).

53. The method of claim 51, wherein the ALS is familial ALS (fALS).

54. The method of claim 35 wherein the subject in need thereof does not exhibit motor impairment but is predisposed to motor impairment.

55. The method of claim 35, wherein the pharmaceutical composition administered parenterally.

56. The method of claim 42, wherein the pharmaceutical composition is administered intravenously, intramuscularly, intracranially, or intrathecally.
